(19) United States
(12) Patent Application Publication
(10) Pub. No.: US 2026/0274810 A1
(43) Pub. Date: Sep. 17, 2026

(54) NOVEL MITOFUSIN INHIBITORS

(71) Applicant: ALBERT EINSTEIN COLLEGE OF MEDICINE, Bronx, NY (US)

(72) Inventors: Evripidis Gavathiotis, Roslyn, NY (US); Emmanouil Zacharloudakis, Bronx, NY (US)

(21) Appl. No.: 19/489,228

(22) PCT Filed: Jun. 5, 2024
(86) PCT No.: PCT/US2024/032586

§ 371 Date: Dec. 1, 2025

Related U.S. Application Data
(60) Provisional application No. 63/506,182, filed on Jun. 5, 2023.

(51) Int. Cl. C07D 295/155; A61K 31/137; A61K 31/167; A61K 31/18; A61K 31/4418; A61K 31/47; A61K 31/505; C07C 215/50; C07C 235/64; C07C 311/29; C07D 213/56; C07D 215/14; C07D 239/26

(52) U.S. Cl. CPC C07D 295/155; A61K 31/137; A61K 31/167; A61K 31/18; A61K 31/4418; A61K 31/47; A61K 31/505; C07C 215/50; C07C 235/64; C07C 311/29; C07D 213/56; C07D 215/14; C07D 239/26

(57) ABSTRACT

Disclosed herein are mitofusin inhibitors which are capable of inducing mitochondrial fission, decreasing mitochondrial respiration, TCA metabolism, and inducing mitochondrial outer membrane permeabilization that leads to caspase activation and DNA damage signaling. Also disclosed are methods of treating diseases or conditions associated with imbalanced mitochondrial dynamics.

### RELATED APPLICATIONS AND CLAIM OF PRIORITY

This patent document claims priority to U.S. Provisional Patent Application No. 63/506,182, filed Jun. 5, 2023, the disclosure of which is fully incorporated into this document by reference.

### TECHNICAL FIELD

Disclosed herein are mitofusin inhibitors and their use in treating various diseases and conditions.

### BACKGROUND

Mitochondria provide eukaryotic cells with a means to generate significant quantities of ATP through oxidative phosphorylation. They are also potentially dangerous organelles because of their abilities to generate reactive oxygen and nitrogen species and mediate cell death. In part because of these liabilities, mitochondria engage in repeated cycles of fission and fusion. These events, referred to in the aggregate as mitochondrial dynamics, are tightly coupled with the elimination of damaged mitochondria through macroautophagy (mitophagy) and with mitochondrial biogenesis, processes which together maintain the health of the mitochondrial collective in a cell.

Mitochondrial fusion is a two step process; the first step requires the fusion of the outer mitochondrial membrane (OMM), which is mediated by mitofusin-1 (MFN1) and mitofusin-2 (MFN2). MFN1 and MFN2 are mediator of mitochondrial fusion. They are mitochondrial membrane proteins that interact with each other to facilitate mitochondrial targeting. The second step requires the fusion of the inner mitochondrial membrane, which is mediated by optic atrophy-1 (OPA1). Loss of either MFN1/2 or OPA1 proteins results in a network of hyper-fragmented mitochondria. While both MFN1 and MFN2 deficient cells display a clearly fragmented mitochondrial network, MFN1 knockout cells display severe mitochondrial fragmentation with formation of small spheres of similar size and MFN2 knockout cells display mitochondrial spheres or ovals of variable but larger size. Overexpression of either MFN1 or MFN2 in wild type cells leads to extensive mitochondrial clustering in the perinuclear area. However, overexpression of MFN1 in MFN2 KO and MFN2 in MFN1 KO cells restores mitochondrial fusion, highlighting the degree of redundancy between the MFN proteins.

Multiple diseases ranging from cancer, heart disease, diabetes, and neurodegenerative disorders, as well as aging, are accompanied by disruption of mitochondrial dynamics. Often this is characterized by an imbalance in mitochondrial fission over fusion.

Thus, there is a need to develop new agents for treating diseases or conditions associated with imbalance in mitochondrial dynamics.

### SUMMARY

Mitofusins reside in the outer mitochondrial membrane and regulate mitochondrial fusion, a physiological process that impacts diverse cellular processes. Mitofusins are activated by conformational changes and subsequently oligomerize to enable mitochondrial fusion. Mitofusin activation increases mitochondrial fusion and functionality, whereas mitofusin inhibition decreases mitochondrial fusion and functionality. Remarkably, mitofusin inhibition also induces minority mitochondrial outer membrane permeabilization (MOMP), followed by sub-lethal caspase-3/7 activation, which induces DNA damage and upregulated DNA damage response genes. This patent disclosure provides compounds that induce fragmented mitochondria and lead to decreased membrane potential, mitochondrial respiration and ATP production.

An aspect of the patent document provides a compound of Formula I and pharmaceutically acceptable salts thereof. The compound is generally represented as follows:

An aspect of the disclosure provides a compound of formula I or a pharmaceutically acceptable salt thereof, which are capable of regulating directly MFN1/2 activity and subsequently mitochondrial fusion. The compound binds directly to the recombinant HR2 domain of MFN2 and in cells to intact protein, decreases the GTP-dependent MFN2 higher-order oligomers, and therefore impedes mitochondrial fusion by directly interfering with the tethering permissive structure of MFNs.

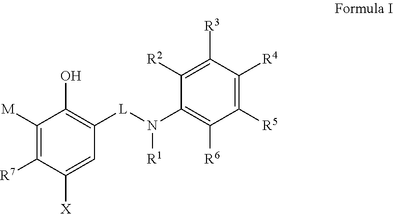

wherein:
M is selected from hydrogen, deuterium, OC₁₋₆alkyl, SC₁₋₆alkyl, CN, OH, SH, COOH, halogen, NO₂, N(R^{m})₂, C(O)OR^{m}, C(O)N(R^{m})₂, C(O)C₁₋₆alkyl, haloC₁₋₆alkyl, haloC₁₋₆alkyleneO, C₁₋₆alkyl, hydroxyC₁₋₆alkyl, dihydroxyC₁₋₁₀alkyl, C₃₋₆cycloalkyl, C(═NC₁₋₆alkyl)C₁₋₆alkyl, OC(O)N(R^{m})₂, C(O)SR^{m}, OC₁₋₆alkyleneOC₁₋₆alkyl, OC₁₋₆alkyleneO-haloC₁₋₆alkyl, SC₁₋₆alkyleneOC₁₋₆alkyl, SC₁₋₆alkyleneSC₁₋₆alkyl, OC₁₋₆alkyleneSC₁₋₆alkyl, SC₁₋₆alkyleneO-haloC₁₋₆alkyl, SC₁₋₆alkyleneS-haloC₁₋₆alkyl, OC₁₋₆alkyleneS-haloC₁₋₆alkyl, C₁₋₆alkylene-CN, OC₁₋₆alkylene-CN, SC₁₋₆alkylene-CN, OC₁₋₆alkylene-N(R^{m})₂, C₂₋₆alkynyl, C₂₋₆alkenyl, SO₂N(R^{m})₂, NR^{m}SO₂C₁₋₆alkyl, C₁₋₆alkylSO₂ (sulfone), S(O)OH, C₁₋₆alkylS(O) (sulfoxide), nitroso, and C₁₋₆alkylOSO₂, or Ar,
Ar is a phenyl, naphthyl, 5-10-membered heteroaryl or 4-8 membered heterocyclyl, wherein the phenyl, naphthyl, heteroaryl or heterocycyl is optionally substituted with one more substituents selected from the group consisting of deuterium, OC₁₋₆alkyl, SC₁₋₆alkyl, CN, OH, SH, COOH, halogen, NO₂, N(R^{m})₂, C(O)OR^{m}, C(O)N(R^{m})₂, C(O)C₁₋₆alkyl, haloC₁₋₆alkyl, haloC₁₋₆alkyleneO, C₁₋₆alkyl, hydroxyC₁₋₆alkyl, dihydroxyC₁₋₁₀alkyl, C₃₋₆cycloalkyl, C(═NC₁₋₆alkyl)C₁₋₆alkyl, OC(O)N(R^{m})₂, C(O)SR^{m}, OC₁₋₆alkyleneOC₁₋₆alkyl, OC₁₋₆alkyleneO-haloC₁₋₆alkyl, SC₁₋₆alkyleneOC₁₋₆alkyl, SC₁₋₆alkyleneSC₁₋₆alkyl, OC₁₋₆alkyleneSC₁₋₆alkyl, SC₁₋₆alkyleneO-haloC₁₋₆alkyl, SC₁₋₆alkyleneS-haloC₁₋₆alkyl, OC₁₋₆alkyleneS-haloC₁₋₆alkyl, C₁₋₆alkylene-CN, OC₁₋₆alkylene-CN, SC₁₋₆alkylene-CN, OC₁₋₆alkylene-N(R^{m})₂, C₂₋₆alkynyl, C₂₋₆alkenyl, SO₂N(R^{m})₂, NR^{m}SO₂C₁₋₆alkyl, C₁₋₆alkylSO₂ (sulfone), S(O)OH, C₁₋₆alkylS(O) (sulfoxide), nitroso, and C₁₋₆alkylOSO₂;
X is halogen;
L is C₁₋₃alkylene optionally substituted with an oxo (═O), SO, SO₂ of OC(═O);
R¹ is hydrogen, C₁₋₆alkyl or C(O)C₁₋₄alkyl,
R² and R³ are independently selected from the group consisting of hydrogen, OC₁₋₆alkyl, SC₁₋₆alkyl, C₁₋₄alkyl, CN, halogen, C₁₋₆alkylene-CN, OC₁₋₆alkylene-CN, haloC₁₋₆alkyl, SC₁₋₆alkylene-CN, C₂₋₆alkynyl, C₂₋₆alkenyl, and C₁₋₆alkylSO₂ (sulfone);
R⁴, R⁵, and R⁶ are independently selected from the group consisting of hydrogen, deuterium, OC₁₋₆alkyl, SC₁₋₆alkyl, CN, OH, SH, halogen, NO₂, N(R^{m})₂, C(O)OR^{m}, C(O)N(R^{m})₂, C(O)C₁₋₆alkyl, haloC₁₋₆alkyl, haloC₁₋₆alkyleneO, C₁₋₆alkyl, hydroxyC₁₋₆alkyl, dihydroxyC₁₋₁₀alkyl, C₃₋₆cycloalkyl, C(═NC₁₋₆alkyl)C₁₋₆alkyl, OC(O)N(R^{m})₂, C(O)SR^{m}, OC₁₋₆alkyleneOC₁₋₆alkyl, OC₁₋₆ alkyleneO-haloC₁₋₆alkyl, SC₁₋₆alkyleneOC₁₋₆alkyl, SC₁₋₆alkyleneSC₁₋₆alkyl, OC₁₋₆alkyleneSC₁₋₆alkyl, SC₁₋₆alkyleneO-haloC₁₋₆alkyl, SC₁₋₆alkyleneS-haloC₁₋₆alkyl, OC₁₋₆alkyleneS-haloC₁₋₆alkyl, C₁₋₆alkylene-CN, OC₁₋₆alkylene-CN, SC₁₋₆alkylene-CN, OC₁₋₆alkylene-N(R^{m})₂, C₂₋₆alkynyl, C₂₋₆alkenyl, SO₂N(R^{m})₂, NR^{m}SO₂C₁₋₆alkyl, C₁₋₆alkylSO₂ (sulfone), S(O)OH, C₁₋₆alkylS(O) (sulfoxide), nitroso, C₁₋₆alkylOSO₂;
R⁷ is selected from the group consisting of hydrogen, deuterium, OC₁₋₆alkyl, SC₁₋₆alkyl, CN, OH, SH, halogen, NO₂, N(R^{m})₂, C(O)OR^{m}, C(O)N(R^{m})₂, C(O)C₁₋₆alkyl, haloC₁₋₆alkyl, haloC₁₋₆alkyleneO, C₁₋₆alkyl, hydroxyC₁₋₆alkyl, dihydroxyC₁₋₁₀alkyl, C₃₋₆cycloalkyl, C(═NC₁₋₆alkyl)C₁₋₆alkyl, OC(O)N(R^{m})₂, C(O)SR^{m}, OC₁₋₆alkyleneOC₁₋₆alkyl, OC₁₋₆alkyleneO-haloC₁₋₆alkyl, SC₁₋₆ alkyleneOC₁₋₆alkyl, SC₁₋₆alkyleneSC₁₋₆alkyl, OC₁₋₆alkyleneSC₁₋₆alkyl, SC₁₋₆alkyleneO-haloC₁₋₆alkyl, SC₁₋₆alkyleneS-haloC₁₋₆alkyl, OC₁₋₆alkyleneS-haloC₁₋₆alkyl, C₁₋₆alkylene-CN, OC₁₋₆ alkylene-CN, SC₁₋₆alkylene-CN, OC₁₋₆alkylene-N(R^{m})₂, C₂₋₆alkynyl, C₂₋₆alkenyl, SO₂N(R^{m})₂, NR^{m}SO₂C₁₋₆alkyl, C₁₋₆alkylSO₂ (sulfone), S(O)OH, C₁₋₆alkylS(O) (sulfoxide), nitroso, C₁₋₆alkylOSO₂, phenyl, naphthyl, 5-10-membered heteroaryl or 4-8 membered heterocyclyl, wherein the phenyl, naphthyl, heteroaryl or heterocycyl is optionally substituted with one more substituents selected from the group consisting of deuterium, OC₁₋₆alkyl, SC₁₋₆alkyl, CN, OH, SH, COOH, halogen, NO₂, N(R^{m})₂, C(O)OR^{m}, C(O)N(R^{m})₂, C(O)C₁₋₆alkyl, haloC₁₋₆alkyl, haloC₁₋₆alkyleneO, C₁₋₆alkyl, hydroxyC₁₋₆alkyl, dihydroxyC₁₋₁₀alkyl, C₃₋₆cycloalkyl, C(═NC₁₋₆alkyl)C₁₋₆alkyl, OC(O)N(R^{m})₂, C(O)SR^{m}, OC₁₋₆alkyleneOC₁₋₆alkyl, OC₁₋₆alkyleneO-haloC₁₋₆ alkyl, SC₁₋₆alkyleneOC₁₋₆alkyl, SC₁₋₆alkyleneSC₁₋₆alkyl, OC₁₋₆alkyleneSC₁₋₆alkyl, SC₁₋₆ alkyleneO-haloC₁₋₆alkyl, SC₁₋₆alkyleneS-haloC₁₋₆alkyl, OC₁₋₆alkyleneS-haloC₁₋₆alkyl, C₁₋₆alkylene-CN, OC₁₋₆alkylene-CN, SC₁₋₆alkylene-CN, OC₁₋₆alkylene-N(R^{m})₂, C₂₋₆alkynyl, C₂₋₆alkenyl, SO₂N(R^{m})₂, NR^{m}SO₂C₁₋₆alkyl, C₁₋₆alkylSO₂ (sulfone), S(O)OH, C₁₋₆alkylS(O) (sulfoxide), nitroso, and C₁₋₆alkylOSO₂;
optionally, two adjacent substituents of R², R³, R⁴, R⁵, and R⁶ link up to form, together with the phenyl they are attached to, a bicyclic ring, wherein the bicyclic ring is optionally substituted with one more substituents selected from the group consisting of deuterium, OC₁₋₆alkyl, SC₁₋₆alkyl, CN, OH, SH, COOH, halogen, NO₂, N(R^{m})₂, C(O)OR^{m}, C(O)N(R^{m})₂, C(O)C₁₋₆alkyl, haloC₁₋₆alkyl, haloC₁₋₆alkyleneO, C₁₋₆alkyl, hydroxyC₁₋₆alkyl, dihydroxyC₁₋₁₀alkyl, C₃₋₆cycloalkyl, C(═NC₁₋₆alkyl)C₁₋₆alkyl, OC(O)N(R^{m})₂, C(O)SR^{m}, OC₁₋₆alkyleneOC₁₋₆alkyl, OC₁₋₆ alkyleneO-haloC₁₋₆alkyl, SC₁₋₆alkyleneOC₁₋₆alkyl, SC₁₋₆alkyleneSC₁₋₆alkyl, OC₁₋₆alkyleneSC₁₋₆alkyl, SC₁₋₆alkyleneO-haloC₁₋₆alkyl, SC₁₋₆alkyleneS-haloC₁₋₆alkyl, OC₁₋₆alkyleneS-haloC₁₋₆alkyl, C₁₋₆alkylene-CN, OC₁₋₆alkylene-CN, SC₁₋₆alkylene-CN, OC₁₋₆alkylene-N(R^{m})₂, C₂₋₆alkynyl, C₂₋₆alkenyl, SO₂N(R^{m})₂, NR^{m}SO₂C₁₋₆alkyl, C₁₋₆alkylSO₂ (sulfone), S(O)OH, C₁₋₆alkylS(O) (sulfoxide), nitroso, and C₁₋₆alkylOSO₂;
alternatively, L, NR¹, and R², together with the phenyl attached to NR¹, form a bicyclic ring, wherein the bicyclic ring is optionally substituted with one more substituents selected from the group consisting of deuterium, OC₁₋₆alkyl, SC₁₋₆alkyl, CN, OH, SH, COOH, halogen, NO₂, N(R^{m})₂, C(O)OR^{m}, C(O)N(R^{m})₂, C(O)C₁₋₆alkyl, haloC₁₋₆alkyl, haloC₁₋₆alkyleneO, C₁₋₆alkyl, hydroxyC₁₋₆alkyl, dihydroxyC₁₋₁₀alkyl, C₃₋₆cycloalkyl, C(═NC₁₋₆alkyl)C₁₋₆alkyl, OC(O)N(R^{m})₂, C(O)SR^{m}, OC₁₋₆alkyleneOC₁₋₆alkyl, OC₁₋₆alkyleneO-haloC₁₋₆alkyl, SC₁₋₆alkyleneOC₁₋₆alkyl, SC₁₋₆alkyleneSC₁₋₆alkyl, OC₁₋₆alkyleneSC₁₋₆alkyl, SC₁₋₆alkyleneO-haloC₁₋₆alkyl, SC₁₋₆ alkyleneS-haloC₁₋₆alkyl, OC₁₋₆alkyleneS-haloC₁₋₆alkyl, C₁₋₆alkylene-CN, OC₁₋₆alkylene-CN, SC₁₋₆alkylene-CN, OC₁₋₆alkylene-N(R^{m})₂, C₂₋₆alkynyl, C₂₋₆alkenyl, SO₂N(R^{m})₂, NR^{m}SO₂C₁₋₆ alkyl, C₁₋₆alkylSO₂ (sulfone), S(O)OH, C₁₋₆alkylS(O) (sulfoxide), nitroso, and C₁₋₆alkylOSO₂;
and
R^{m} in each instance is independently hydrogen or C₁₋₆alkyl or halo-C₁₋₆alkyl.

In some embodiments, the compound is not any of the following:

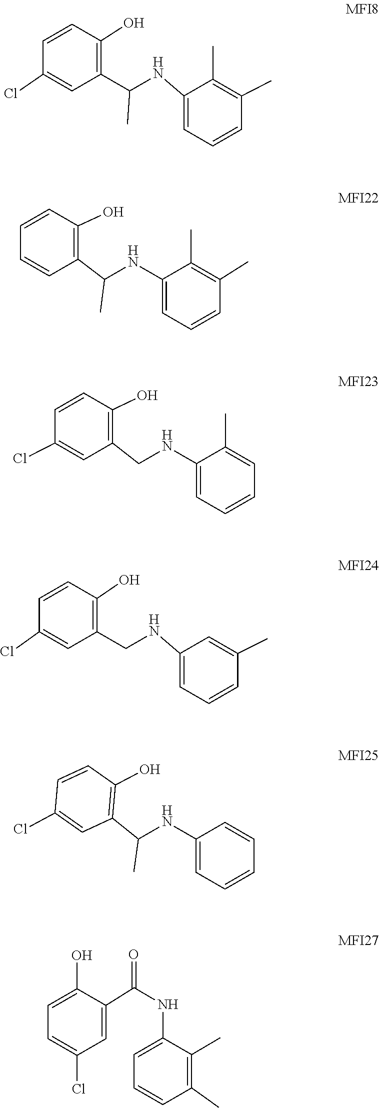 [SMILES: Cc1cccc(NC(C)c2cc(Cl)ccc2O)c1C ; Cc1cccc(NC(C)c2ccccc2O)c1C ; Cc1ccccc1NCc1cc(Cl)ccc1O ; Cc1cccc(NCc2cc(Cl)ccc2O)c1 ; CC(Nc1ccccc1)c1cc(Cl)ccc1O ; Cc1cccc(NC(=O)c2cc(Cl)ccc2O)c1C]

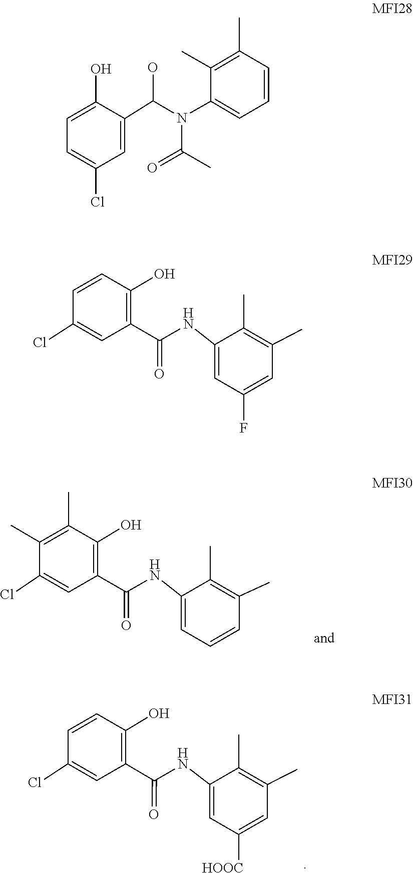

Also provided are pharmaceutical compositions comprising a therapeutically effective amount of a compound of Formula (I) disclosed herein or a pharmaceutically acceptable salt thereof, and a pharmaceutically acceptable excipient.

Another aspect provides a method for treating a disease or condition. The method includes administering to a subject in need thereof a compound of formula (I), a pharmaceutically acceptable salt thereof, or a pharmaceutical composition thereof.

Another aspect provides a method for inhibiting mitofusin-mediated mitochondrial fusion. The method includes contacting a cell with an effective amount of the compound of formula (I) or a pharmaceutically acceptable salt thereof or a pharmaceutical composition thereof.

### DESCRIPTION OF THE DRAWINGS

FIG. 1 shows the activities of some example compounds. MFI8, MFI43 and MFI38 concentration-responsively decreased Mito AR in MEFs. Cells were treated with MFIs at the indicated concentrations for 6 h. Compounds MFI8, MFI43, and MFI38 have an IC50 value of 4.062, 1.793, and 2.343 μM, respectively.

FIG. 2 shows the liver microsome stability of some example compounds. MFI38, MFI43, and MFI56 have increased half life (t½) in a mouse liver microsome assay. This result suggests that MFI38, MFI43, and MFI56 are more stable compounds with improved bioavailability.

FIG. 3 shows the synthesis of an example compound, MFI38.

FIG. 4 shows the synthesis of example compounds MFI45 and MFI46.

FIG. 5 shows an example of the synthesis of a compound under Formula II.

FIG. 6 shows an example of the synthesis of a compound under Formula II.

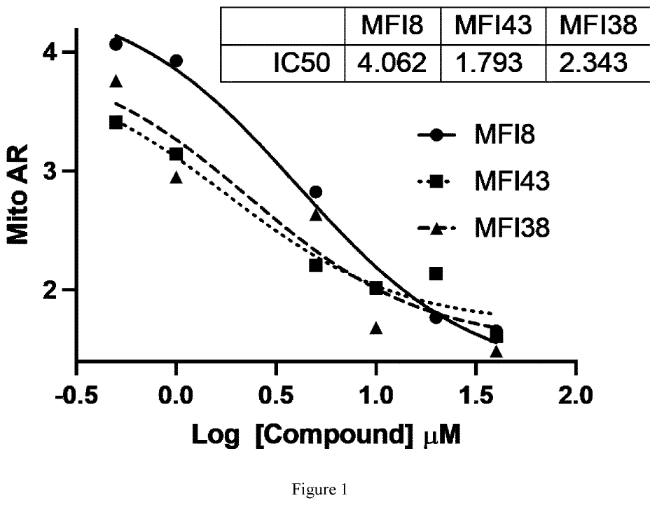

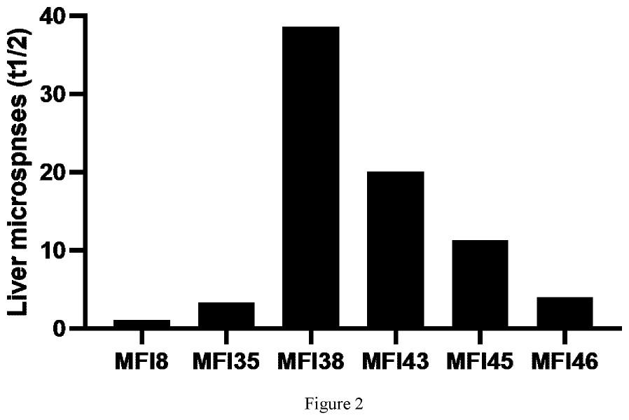

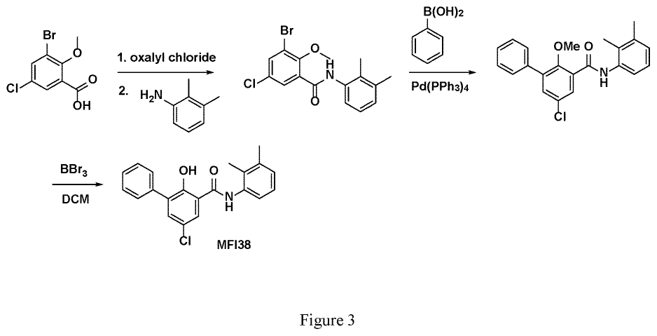

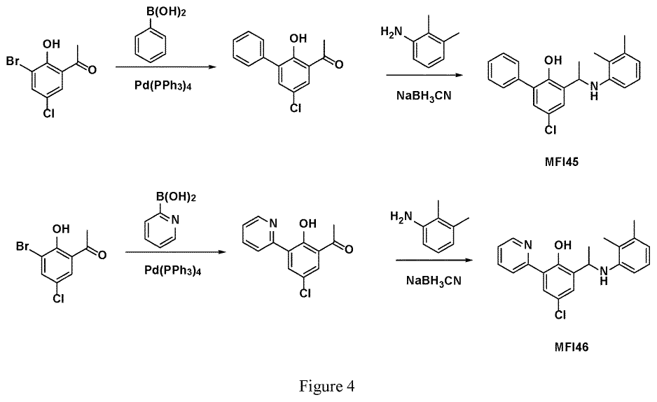

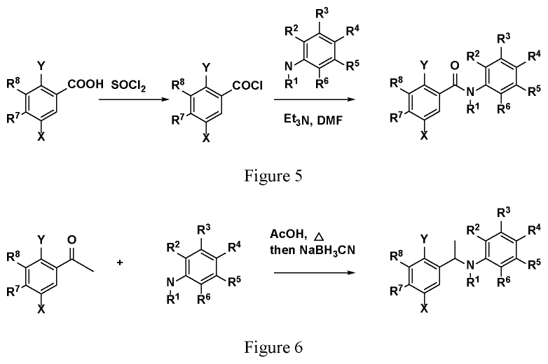

### DETAILED DESCRIPTION

This patent document discloses mitofusin inhibitors and their effects in inducing mitochondrial fission, decreasing mitochondrial respiration, TCA metabolism, and inducing mitochondrial outer membrane permeabilization that leads to caspase activation and DNA damage signaling. Multiple diseases including for example cancer, heart disease, diabetes, and neurodegenerative disorders, as well as aging, are accompanied by disruption of mitochondrial dynamics. Accordingly, the compounds disclosed herein possess great therapeutic potential for treating diseases associated with imbalance in mitochondrial dynamics.

While the following text may reference or exemplify specific embodiments of a compound, substituent, or use thereof, it is not intended to limit the scope of the compound, substituent or its use to such particular references or examples. Various modifications may be made by those skilled in the art, in view of scientific and practical considerations, such as replacement of a substituent or treatment of other diseases.

The articles “a” and “an” as used herein refer to “one or more” or “at least one,” unless otherwise indicated. That is, reference to any element or component of an embodiment by the indefinite article “a” or “an” does not exclude the possibility that more than one element or component is present.

The term “acyl” refers to —C(O)CH₃, —C(O)CH₂CH₃, —C(O)CH₂CH₂CH₃, or —C(O)CH₂CH₂CH₂CH₃.

The term “alkyl” refers to a hydrocarbon or a hydrocarbon chain which may be either straight-chained or branched. The term “C₁₋₆ alkyl” refers to alkyl groups having 1, 2, 3, 4, 5 or 6 carbon atoms. Non-limiting examples include groups such as CH₃, (CH₂)₂CH₃, CH₂CH(CH₃)CH₃, and the like. Similarly, the term “C₂₋₅ alkyl” refers to alkyl groups having 2, 3, 4 or 5 carbon atoms.

The term “alkylene” refers to a divalent hydrocarbon or a hydrocarbon chain which may be either straight-chained or branched. Non-limiting examples include groups such as CH₂, (CH₂)₂CH₂, CH₂CH(CH₃)CH₂, and the like. A C₁₋₃alkylene includes alkylenes with 1, 2 or 3 carbons such as CH₂, (CH₂)₂, CHCH₃,(CH₂)₃, and CH(CH₃)CH₂.

The term “cycloalkyl” refers to saturated and partially unsaturated cyclic hydrocarbon groups having 3 to 12 ring carbons, for example 3 to 8 carbons, and as a further example 3 to 6 carbons, wherein the cycloalkyl group additionally is optionally substituted. Examples of cycloalkyl groups include, without limitation, cyclopropyl, cyclobutyl, cyclopentyl, cyclopentenyl, cyclohexyl, cyclohexenyl, cycloheptyl, and cyclooctyl.

The term “aryl” group refers to a C₆₋₁₄ aromatic moiety comprising one to three aromatic rings, which is optionally substituted. Examples of aryl groups include, without limitation, phenyl, naphthyl, anthracenyl, fluorenyl, and dihydrobenzofuranyl.

The term “alkeny” refers to a carbon chain containing a carbon-carbon double bond moiety. Non-limiting examples of alkenyl groups include ethylenyl, 1-propenyl, allyl and 2-butenyl.

The term “alkynyl” group refers to a caron chain containing a carbon-carbon triple bond moiety. Non-limiting examples of alkynyl groups include ethynyl, 1-propanyl, propargyl and 2-butynyl.

The term “haloalkyl” refers to a C₆₋₁₀alkyl chain, straight or branched, in which one or more hydrogen has been replaced by a halogen. Non-limiting examples of haloalkyls include CHF₂, CFH₂, CF₃, CH₂CHF₂, CH₂CH₂Cl, CH₂CF₃, and CH₂CH₂F. In some embodiments, the alkyl in haloalkyl has 1, 2, 3 or 4 carbons.

The term “heteroalkyl” refers to a C₆₋₁₀alkyl group, straight or branched, wherein one or more carbon atoms in the chain are replaced by one or more heteroatoms selected from the group consisting of O, S, N and NR^{m}. In some embodiments, the alkyl in heteroalkyl has 1 to 10 carbons. In some embodiments, the alkyl in heteroalkyl has 2, 3, 4 or more than 2 carbons.

The term “hydroxyalkyl” refers to a C₆₋₁₀alkyl chain, straight or branched, wherein a carbon is substituted with a hydroxyl group. The carbon the hydroxyl is attached to is a primary carbon or secondary carbon. In some embodiments, the alkyl in hydroxylalkyl has 2, 3, 4 or more than 2 carbons.

The term “dihydroxyalkyl” refers to a C₂₋₁₀alkyl chain, straight or branched, wherein two carbons are each substituted with a hydroxyl group. In some embodiments, the alkyl in dihydroxylalkyl has 2, 3, 4 or more than 2 carbons.

The term “heterocyclyl” or “heterocyclic” group is a ring structure having from about 3 to about 12 atoms, for example 4 to 8 atoms, wherein one or more atoms are selected from the group consisting of N, O, and S, the remainder of the ring atoms being carbon. The heterocyclyl may be a monocyclic, a bicyclic, a spirocyclic or a bridged ring system. Examples of heterocyclic groups include, without limitation, epoxy, azetidinyl, aziridinyl, azocanyl, azepanyl, diazepanyl, dihydrofuranyl, tetrahydrofuranyl, tetrahydropyranyl, oxazepanyl, pyrrolidinyl, pyrrolidinonyl, piperidinyl, piperazinyl, imidazolidinyl, thiazolidinyl, thiooxazepanyl, dithianyl, trithianyl, dioxolanyl, oxazolidinyl, oxazolidinonyl, decahydroquinolinyl, piperidonyl, 4-piperidinonyl, thiomorpholinyl, thiomorpholinyl 1,1 dioxide, morpholinyl, oxazepanyl, azabicyclohexanes, azabicycloheptanes and oxa azabiocycloheptanes. Specifically excluded from the scope of this term are compounds having adjacent annular O and/or S atoms.

The term “heteroaryl” refers to groups having 5 to 14 ring atoms, preferably 5, 6, 9, or 10 ring atoms; having 6, 10, or 14 π electrons shared in a cyclic array; and having, in addition to carbon atoms, from one to three heteroatoms per ring selected from the group consisting of N, O, and S. Examples of heteroaryl groups include acridinyl, azocinyl, benzimidazolyl, benzofuranyl, benzothiofuranyl, benzothiophenyl, benzoxazolyl, benzthiazolyl, benztriazolyl, benztetrazolyl, benzisoxazolyl, benzisothiazolyl, benzimidazolinyl, carbazolyl, 4aH-carbazolyl, carbolinyl, chromanyl, chromenyl, cinnolinyl, furanyl, furazanyl, imidazolinyl, imidazolyl, 1H-indazolyl, indolenyl, indolinyl, indolizinyl, indolyl, 3H-indolyl, isobenzofuranyl, isochromanyl, isoindazolyl, isoindolinyl, isoindolyl, isoquinolinyl, isothiazolyl, isoxazolyl, methylenedioxyphenyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxazolidinyl, pyrimidinyl, phenanthridinyl, phenanthrolinyl, phenazinyl, phenothiazinyl, phenoxathiinyl, phenoxazinyl, phthalazinyl, piperonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolinyl, 2H-pyrrolyl, pyrrolyl, quinazolinyl, quinolinyl, 4H-quinolizinyl, quinoxalinyl, quinuclidinyl, tetrahydroisoquinolinyl, tetrahydroquinolinyl, tetrazolyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl, triazinyl, 1,2,3-triazolyl, 1,2,4-triazolyl, 1,2,5-triazolyl, 1,3,4-triazolyl, and xanthenyl.

The term “halogen” refers to F, Cl, Br or I.

The term “subject” refers to humans or animals including for example sheep, horses, cattle, pigs, dogs, cats, rats, mice, birds, and reptiles. Preferably, the subject is a human or other mammal.

The term “effective amount” or “therapeutically effective amount” of a compound is an amount that is sufficient to ameliorate, or in some manner reduce a symptom or stop or reverse progression of a condition, or negatively modulate or inhibit activity. Such amount may be administered as a single dosage or may be administered according to a regimen, whereby it is effective.

The term “hydrogen bond donor” refers to a group containing a hydrogen, which can be form a hydrogen bond with another electronegative atom such as F, N or O. Non-limiting examples of hydrogen bond donor include OH and NH₂, which can share its hydrogen with electron rich atoms to form a hydrogen bond.

The term “hydrogen bond acceptor” refers to a group or atom rich in electrons, which can form a hydrogen bond with a hydrogen bond donor. Non-limiting examples of hydrogen bond acceptor include O, N and F.

The term “pharmaceutically acceptable” refers to those compounds, materials, compositions, and/or dosage forms which are, within the scope of sound medical judgment, suitable for use in contact with the tissues, organs, and/or bodily fluids of human beings and animals without excessive toxicity, irritation, allergic response, or other problems or complications commensurate with a reasonable benefit/risk ratio.

The term “pharmaceutically acceptable carrier” refers to a chemical compound that facilitates the delivery or incorporation of a compound or therapeutic agent into cells or tissues.

The term “pharmaceutically acceptable salts” means salts of compounds of the present invention which are pharmaceutically acceptable, as defined above, and which possess the desired pharmacological activity. Non-limiting examples of such salts include acid addition salts formed with inorganic acids such as hydrochloric acid, hydrobromic acid, sulfuric acid, nitric acid, and phosphoric acid; or with organic acids such as 1,2-ethanedisulfonic acid, 2-hydroxyethanesulfonic acid, 2-naphthalenesulfonic acid, 3-phenylpropionic acid, 4,4′-methylenebis(3-hydroxy-2-ene-1-carboxylic acid), 4-methylbicyclo[2.2.2]oct-2-ene-1-carboxylic acid, acetic acid, aliphatic mono- and dicarboxylic acids, aliphatic sulfuric acids, aromatic sulfuric acids, benzenesulfonic acid, benzoic acid, camphorsulfonic acid, carbonic acid, cinnamic acid, citric acid, cyclopentanepropionic acid, ethanesulfonic acid, fumaric acid, glucoheptonic acid, gluconic acid, glutamic acid, glycolic acid, heptanoic acid, hexanoic acid, hydroxynaphthoic acid, lactic acid, laurylsulfuric acid, maleic acid, malic acid, malonic acid, mandelic acid, methanesulfonic acid, muconic acid, o-(4-hydroxybenzoyl)benzoic acid, oxalic acid, p-chlorobenzenesulfonic acid, phenyl-substituted alkanoic acids, propionic acid, p-toluenesulfonic acid, pyruvic acid, salicylic acid, stearic acid, succinic acid, tannic acid, tartaric acid, tertiarybutylacetic acid, and trimethylacetic acid. Pharmaceutically acceptable salts also include base addition salts which may be formed when acidic protons present are capable of reacting with inorganic or organic bases. Acceptable inorganic bases include sodium hydroxide, sodium carbonate, potassium hydroxide, aluminum hydroxide and calcium hydroxide. Non-limiting examples of acceptable organic bases include ethanolamine, diethanolamine, ethylenediamine, triethanolamine, tromethamine, and N-methylglucamine. It should be recognized that the particular anion or cation forming a part of any salt of this invention is not critical, so long as the salt, as a whole, is pharmacologically acceptable. Additional examples of pharmaceutically acceptable salts and their methods of preparation and use are presented in Handbook of Pharmaceutical Salts: Properties, and Use (P. H. Stahl & C. G. Wermuth eds., Verlag Helvetica Chimica Acta, 2002).

The term “pharmaceutical composition” refers to a mixture of a compound disclosed herein with other chemical components, such as diluents or additional carriers. The pharmaceutical composition facilitates administration of the compound to an organism. Multiple techniques of administering a pharmaceutical composition exist in the art including, but not limited to, oral, injection, aerosol, parenteral, intranasal, sublingual, inhalational, and topical administration. In some embodiments, pharmaceutically acceptable salts of the compounds disclosed herein are provided.

The term “treating” or “treatment” of any disease or condition refers, in some embodiments, to ameliorating the disease or disorder (i.e., arresting or reducing the development of the disease or at least one of the clinical signs and symptoms thereof). In some embodiments “treating” or “treatment” refers to ameliorating at least one physical parameter, which may not be discernible by the subject. In some embodiments, “treating” or “treatment” refers to modulating the disease or disorder, either physically, (e.g., stabilization of a discernible symptom), physiologically, (e.g., stabilization of a physical parameter), or both. In some embodiments, “treating” or “treatment” refers to delaying the onset of the disease or disorder, or even preventing the same. “Prophylactic treatment” is to be construed as any mode of treatment that is used to prevent progression of the disease or is used for precautionary purpose for persons at risk of developing the condition.

Mitochondria fuse, divide and interact with other organelle structures to regulate cellular fitness and fate while they produce the majority of energy to sustain cellular activity. They are highly dynamic organelles constantly undergoing the physiological process of fusion and fission which regulates mitochondrial morphology and dynamics. Among different cell types or within the same type of cells, mitochondria morphology varies among small spheres, short rods or long tubules. This dynamism allows mitochondria to exchange components (e.g., lipid membranes, proteins), promote repair and removal of defective mitochondria, thus maintaining mitochondrial function and quality. Furthermore, mitochondrial fusion and fission enable mitochondria to meet cellular energy demands in response to environmental stimuli. Fused mitochondria often lead to increased oxidative phosphorylation and mitochondrial membrane potential. In contrast, fragmented mitochondria often correlate with reduced function, decreased mitochondrial membrane potential and oxidative phosphorylation.

### Compounds

An aspect of the disclosure provides a compound of formula I or a pharmaceutically acceptable salt thereof, which are capable of regulating directly MFN1/2 activity and subsequently mitochondrial fusion. The compound binds directly to the recombinant HR2 domain of MFN2 and in cells to intact protein, decreases the GTP-dependent MFN2 higher-order oligomers, and therefore impedes mitochondrial fusion by directly interfering with the tethering permissive structure of MFNs.

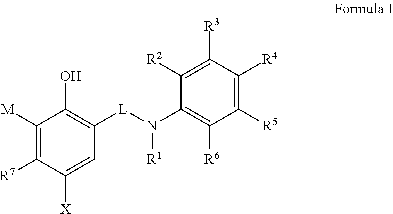

wherein:
M is selected from hydrogen, deuterium, OC₁₋₆alkyl, SC₁₋₆alkyl, CN, OH, SH, COOH, halogen, NO₂, N(R^{m})₂, C(O)OR^{m}, C(O)N(R^{m})₂, C(O)C₁₋₆alkyl, haloC₁₋₆alkyl, haloC₁₋₆alkyleneO, C₁₋₆alkyl, hydroxyC₁₋₆alkyl, dihydroxyC₁₋₁₀alkyl, C₃₋₆cycloalkyl, C(═NC₁₋₆alkyl)C₁₋₆alkyl, OC(O)N(R^{m})₂, C(O)SR^{m}, OC₁₋₆alkyleneOC₁₋₆alkyl, OC₁₋₆alkyleneO-haloC₁₋₆alkyl, SC₁₋₆alkyleneOC₁₋₆alkyl, SC₁₋₆ alkyleneSC₁₋₆alkyl, OC₁₋₆alkyleneSC₁₋₆alkyl, SC₁₋₆alkyleneO-haloC₁₋₆alkyl, SC₁₋₆alkyleneS-haloC₁₋₆alkyl, OC₁₋₆alkyleneS-haloC₁₋₆alkyl, C₁₋₆alkylene-CN, OC₁₋₆alkylene-CN, SC₁₋₆alkylene-CN, OC₁₋₆alkylene-N(R^{m})₂, C₂₋₆alkynyl, C₂₋₆alkenyl, SO₂N(R^{m})₂, NR^{m}SO₂C₁₋₆alkyl, C₁₋₆alkylSO₂ (sulfone), S(O)OH, C₁₋₆alkylS(O) (sulfoxide), nitroso, and C₁₋₆alkylOSO₂, or Ar, wherein Aris a phenyl, naphthyl, 5-10-membered heteroaryl or 4-8 membered heterocyclyl, wherein the phenyl, naphthyl, heteroaryl or heterocycyl is optionally substituted with one more substituents selected from the group consisting of deuterium, OC₁₋₆alkyl, SC₁₋₆alkyl, CN, OH, SH, COOH, halogen, NO₂, N(R^{m})₂, C(O)OR^{m}, C(O)N(R^{m})₂, C(O)C₁₋₆alkyl, haloC₁₋₆alkyl, haloC₁₋₆alkyleneO, C₁₋₆alkyl, hydroxyC₁₋₆alkyl, dihydroxyC₁₋₁₀alkyl, C₃₋₆cycloalkyl, C(═NC₁₋₆alkyl)C₁₋₆alkyl, OC(O)N(R^{m})₂, C(O)SR^{m}, OC₁₋₆alkyleneOC₁₋₆alkyl, OC₁₋₆alkyleneO-haloC₁₋₆alkyl, SC₁₋₆alkyleneOC₁₋₆alkyl, SC₁₋₆ alkyleneSC₁₋₆alkyl, OC₁₋₆alkyleneSC₁₋₆alkyl, SC₁₋₆alkyleneO-haloC₁₋₆alkyl, SC₁₋₆alkyleneS-haloC₁₋₆alkyl, OC₁₋₆alkyleneS-haloC₁₋₆alkyl, C₁₋₆alkylene-CN, OC₁₋₆alkylene-CN, SC₁₋₆alkylene-CN, OC₁₋₆alkylene-N(R^{m})₂, C₂₋₆alkynyl, C₂₋₆alkenyl, SO₂N(R^{m})₂, NR^{m}SO₂C₁₋₆alkyl, C₁₋₆alkylSO₂ (sulfone), S(O)OH, C₁₋₆alkylS(O) (sulfoxide), nitroso, and C₁₋₆alkylOSO₂;
X is halogen;
L is C₁₋₃alkylene optionally substituted with an oxo (═O), SO, SO₂ or OC(═O);
R¹ is hydrogen, C₁₋₆alkyl or C(O)C₁₋₄alkyl,
alternatively, L, NR¹, and R², together with the phenyl attached to NR¹, form a bicyclic ring, wherein the bicyclic ring is optionally substituted with one more substituents selected from the group consisting of deuterium, OC₁₋₆alkyl, SC₁₋₆alkyl, CN, OH, SH, COOH, halogen, NO₂, N(R^{m})₂, C(O)OR^{m}, C(O)N(R^{m})₂, C(O)C₁₋₆alkyl, haloC₁₋₆alkyl, haloC₁₋₆alkyleneO, C₁₋₆alkyl, hydroxyC₁₋₆alkyl, dihydroxyC₁₋₁₀alkyl, C₃₋₆cycloalkyl, C(═NC₁₋₆alkyl)C₁₋₆alkyl, OC(O)N(R^{m})₂, C(O)SR^{m}, OC₁₋₆alkyleneOC₁₋₆alkyl, OC₁₋₆alkyleneO-haloC₁₋₆alkyl, SC₁₋₆alkyleneOC₁₋₆alkyl, SC₁₋₆alkyleneSC₁₋₆alkyl, OC₁₋₆alkyleneSC₁₋₆alkyl, SC₁₋₆alkyleneO-haloC₁₋₆alkyl, SC₁₋₆ alkyleneS-haloC₁₋₆alkyl, OC₁₋₆alkyleneS-haloC₁₋₆alkyl, C₁₋₆alkylene-CN, OC₁₋₆alkylene-CN, SC₁₋₆alkylene-CN, OC₁₋₆alkylene-N(R^{m})₂, C₂₋₆alkynyl, C₂₋₆alkenyl, SO₂N(R^{m})₂, NR^{m}SO₂C₁₋₆ alkyl, C₁₋₆alkylSO₂(sulfone), S(O)OH, C₁₋₆alkylS(O) (sulfoxide), nitroso, and C₁₋₆alkylOSO₂;
R² and R³ are independently selected from the group consisting of hydrogen, OC₁₋₆alkyl, SC₁₋₆alkyl, C₁₋₄alkyl, CN, halogen, C₁₋₆alkylene-CN, OC₁₋₆alkylene-CN, haloC₁₋₆alkyl, SC₁₋₆alkylene-CN, C₂₋₆alkynyl, C₂₋₆alkenyl, and C₁₋₆alkylSO₂ (sulfone);
R⁴, R⁵, and R⁶ are independently selected from the group consisting of hydrogen, deuterium, OC₁₋₆alkyl, SC₁₋₆alkyl, CN, OH, SH, halogen, NO₂, N(R^{m})₂, C(O)OR^{m}, C(O)N(R^{m})₂, C(O)C₁₋₆alkyl, haloC₁₋₆alkyl, haloC₁₋₆alkyleneO, C₁₋₆alkyl, hydroxyC₁₋₆alkyl, dihydroxyC₁₋₁₀alkyl, C₃₋₆cycloalkyl, C(═NC₁₋₆alkyl)C₁₋₆alkyl, OC(O)N(R^{m})₂, C(O)SR^{m}, OC₁₋₆alkyleneOC₁₋₆alkyl, OC₁₋₆ alkyleneO-haloC₁₋₆alkyl, SC₁₋₆alkyleneOC₁₋₆alkyl, SC₁₋₆alkyleneSC₁₋₆alkyl, OC₁₋₆alkyleneSC₁₋₆alkyl, SC₁₋₆alkyleneO-haloC₁₋₆alkyl, SC₁₋₆alkyleneS-haloC₁₋₆alkyl, OC₁₋₆alkyleneS-haloC₁₋₆alkyl, C₁₋₆alkylene-CN, OC₁₋₆alkylene-CN, SC₁₋₆alkylene-CN, OC₁₋₆alkylene-N(R^{m})₂, C₂₋₆alkynyl, C₂₋₆alkenyl, SO₂N(R^{m})₂, NR^{m}SO₂C₁₋₆alkyl, C₁₋₆alkylSO₂ (sulfone), S(O)OH, C₁₋₆alkylS(O) (sulfoxide), nitroso, C₁₋₆alkylOSO₂;
R⁷ is selected from the group consisting of hydrogen, deuterium, OC₁₋₆alkyl, SC₁₋₆alkyl, CN, OH, SH, halogen, NO₂, N(R^{m})₂, C(O)OR^{m}, C(O)N(R^{m})₂, C(O)C₁₋₆alkyl, haloC₁₋₆alkyl, haloC₁₋₆alkyleneO, C₁₋₆alkyl, hydroxyC₁₋₆alkyl, dihydroxyC₁₋₁₀alkyl, C₃₋₆cycloalkyl, C(═NC₁₋₆alkyl)C₁₋₆alkyl, OC(O)N(R^{m})₂, C(O)SR^{m}, OC₁₋₆alkyleneOC₁₋₆alkyl, OC₁₋₆alkyleneO-haloC₁₋₆alkyl, SC₁₋₆ alkyleneOC₁₋₆alkyl, SC₁₋₆alkyleneSC₁₋₆alkyl, OC₁₋₆alkyleneSC₁₋₆alkyl, SC₁₋₆alkyleneO-haloC₁₋₆ alkyl, SC₁₋₆alkyleneS-haloC₁₋₆alkyl, OC₁₋₆alkyleneS-haloC₁₋₆alkyl, C₁₋₆alkylene-CN, OC₁₋₆ alkylene-CN, SC₁₋₆alkylene-CN, OC₁₋₆alkylene-N(R^{m})₂, C₂₋₆alkynyl, C₂₋₆alkenyl, SO₂N(R^{m})₂, NR^{m}SO₂C₁₋₆alkyl, C₁₋₆alkylSO₂ (sulfone), S(O)OH, C₁₋₆alkylS(O) (sulfoxide), nitroso, C₁₋₆alkylOSO₂, phenyl, naphthyl, 5-10-membered heteroaryl or 4-8 membered heterocyclyl, wherein the phenyl, naphthyl, heteroaryl or heterocycyl is optionally substituted with one more substituents selected from the group consisting of deuterium, OC₁₋₆alkyl, SC₁₋₆alkyl, CN, OH, SH, COOH, halogen, NO₂, N(R^{m})₂, C(O)OR^{m}, C(O)N(R^{m})₂, C(O)C₁₋₆alkyl, haloC₁₋₆alkyl, haloC₁₋₆alkyleneO, C₁₋₆alkyl, hydroxyC₁₋₆alkyl, dihydroxyC₁₋₁₀alkyl, C₃₋₆cycloalkyl, C(═NC₁₋₆alkyl)C₁₋₆alkyl, OC(O)N(R^{m})₂, C(O)SR^{m}, OC₁₋₆alkyleneOC₁₋₆alkyl, OC₁₋₆alkyleneO-haloC₁₋₆alkyl, SC₁₋₆alkyleneOC₁₋₆alkyl, SC₁₋₆alkyleneSC₁₋₆alkyl, OC₁₋₆alkyleneSC₁₋₆alkyl, SC₁₋₆ alkyleneO-haloC₁₋₆alkyl, SC₁₋₆alkyleneS-haloC₁₋₆alkyl, OC₁₋₆alkyleneS-haloC₁₋₆alkyl, C₁₋₆alkylene-CN, OC₁₋₆alkylene-CN, SC₁₋₆alkylene-CN, OC₁₋₆alkylene-N(R^{m})₂, C₂₋₆alkynyl, C₂₋₆alkenyl, SO₂N(R^{m})₂, NR^{m}SO₂C₁₋₆alkyl, C₁₋₆alkylSO₂ (sulfone), S(O)OH, C₁₋₆alkylS(O) (sulfoxide), nitroso, and C₁₋₆alkylOSO₂
optionally, two adjacent substituents of R², R³, R⁴, R⁵, and R⁶ link up to form, together with the phenyl they are attached to, a bicyclic ring, wherein the bicyclic ring is optionally substituted with one more substituents selected from the group consisting of deuterium, OC₁₋₆alkyl, SC₁₋₆alkyl, CN, OH, SH, COOH, halogen, NO₂, N(R^{m})₂, C(O)OR^{m}, C(O)N(R^{m})₂, C(O)C₁₋₆alkyl, haloC₁₋₆alkyl, haloC₁₋₆alkyleneO, C₁₋₆alkyl, hydroxyC₁₋₆alkyl, dihydroxyC₁₋₁₀alkyl, C₃₋₆cycloalkyl, C(═NC₁₋₆alkyl)C₁₋₆alkyl, OC(O)N(R^{m})₂, C(O)SR^{m}, OC₁₋₆alkyleneOC₁₋₆alkyl, OC₁₋₆ alkyleneO-haloC₁₋₆alkyl, SC₁₋₆alkyleneOC₁₋₆alkyl, SC₁₋₆alkyleneSC₁₋₆alkyl, OC₁₋₆alkyleneSC₁₋₆alkyl, SC₁₋₆alkyleneO-haloC₁₋₆alkyl, SC₁₋₆alkyleneS-haloC₁₋₆alkyl, OC₁₋₆alkyleneS-haloC₁₋₆alkyl, C₁₋₆alkylene-CN, OC₁₋₆alkylene-CN, SC₁₋₆alkylene-CN, OC₁₋₆alkylene-N(R^{m})₂, C₂₋₆alkynyl, C₂₋₆alkenyl, SO₂N(R^{m})₂, NR^{m}SO₂C₁₋₆alkyl, C₁₋₆alkylSO₂ (sulfone), S(O)OH, C₁₋₆alkylS(O) (sulfoxide), nitroso, and C₁₋₆alkylOSO₂
and
R^{m} in each instance is independently hydrogen or C₁₋₆alkyl or halo-C₁₋₆alkyl.

In some embodiments, Ar is phenyl, 6-membered heteroaryl or 6-membered heterocyclyl, each of which is optionally substituted.

In some embodiments, X is fluorine or chlorine. In some embodiments, X is chlorine.

In some embodiments, R¹ is hydrogen or methyl. In some embodiments, R¹ is C(O)C₁₋₄alkyl (e.g. (CO)Me).

In some embodiments, R² and R³ are independently selected from the group consisting of C₁₋₄alkyl, CN, halo-C₁₋₄alkyl and halogen. In some embodiments, R² and R³ are independently C₁₋₃alkyl. R⁴, R⁵, and R⁶ are independently selected from the group consisting of hydrogen, OH, SH, halogen, NO₂, N(R^{m})₂, C(O)OR^{m}, and C(O)N(R^{m})₂. In some embodiments, R⁴ and R⁶ are each hydrogen and R⁵ is hydrogen or COOH. In some embodiments, R⁷ is hydrogen or C₁₋₃alkyl. In some embodiments, at least one of R², R³, R⁴, R⁵, and R⁶ is halo-C₁₋₄alkyl. In some embodiments, at least one of R², R³, R⁴, R⁵, and R⁶ is halogen. In some embodiments, R² and R³ are independently C₁₋₃alkyl or halo-C₁₋₄alkyl. In some embodiments, both R² and R³ are C₁₋₃alkyl (e.g. methyl, ethyl). In some embodiments, both R² and R³ are halo-C₁₋₂alkyl (e.g. CF₃). In some embodiments, at least one of R⁴ and R⁵ is halogen or halo-C₁₋₂alkyl.

In some embodiments, one or both of R² and R³ are independently C₁₋₄alkyl (e.g. methyl, ethyl or isopropyl) or halo-C₁₋₂alkyl (e.g. CF₃ or CHF₂). In some embodiments, R⁴ is C₁₋₄alkyl or halo-C₁₋₂alkyl. In some embodiments, R⁵ is C₁₋₄alkyl or halo-C₁₋₂alkyl. In some embodiments, R⁶ is C₁₋₄alkyl or halo-C₁₋₂alkyl.

In some embodiments, Ar is phenyl optionally substituted with one or more substituents selected from the group consisting of C₁₋₄alkyl OC₁₋₄alkyl, CN, halogen, N(R^{m})₂, C(O)OR^{m}, and C(O)N(R^{m}).

In some embodiments, Ar is pyridine or pyrimine, wherein the pyridine or pyrimine is optionally substituted with one or more substituents selected from the group consisting of C₁₋₄alkyl OC₁₋₄alkyl, CN, halogen, N(R^{m})₂, C(O)OR^{m}, and C(O)N(R^{m}).

In some embodiments, L is CH₂, CHCH₃, CO, or SO2. In some embodiments, L is C(O). In some embodiments, R¹ is H.

In some embodiments, L is CH₂, CHCH₃ or CHCH₂CH₃. In some embodiments, R¹ is H.

In some embodiments, L and NR¹, together with the phenyl attached to NR¹, form a bicyclic ring, which is optionally substituted. In some embodiments, the optional substituent is selected from OC₁₋₆alkyl, SC₁₋₆alkyl, CN, OH, SH, COOH, halogen, NO₂, N(R^{m})₂, C(O)OR^{m}, C(O)N(R^{m})₂, C(O)C₁₋₆alkyl, haloC₁₋₆alkyl, haloC₁₋₆alkyleneO, C₁₋₆alkyl, hydroxyC₁₋₆alkyl, dihydroxyC₁₋₁₀alkyl, and C₃₋₆cycloalkyl. In some embodiments, the bicyclic ring is quinoline. In some embodiments, the bicyclic ring is indole.

In some embodiments, the ortho substituent of Ar is selected from the group consisting of OC₁₋₆alkyl, SC₁₋₆alkyl, C₁₋₄alkyl, CN, halogen, haloC₁₋₆alkyl, C₂₋₆alkynyl, and C₂₋₆alkenyl. In some embodiments, ortho substituent is C₁₋₄alkyl.

In some embodiments, the ortho substituent of the phenyl (bonded to NR¹) is selected from the group consisting of OC₁₋₆alkyl, SC₁₋₆alkyl, C₁₋₄alkyl, CN, halogen, haloC₁₋₆alkyl, C₂₋₆alkynyl, and C₂₋₆alkenyl. In some embodiments, ortho substituent is C₁₋₄alkyl.

In some embodiments, the compound of Formula I is represented by Formula I-a,

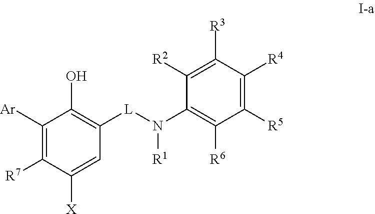

Wherein
L is C₁₋₃alkylene, SO2 or CO;
R¹ is hydrogen or C₁₋₆alkyl;
R², R³, R⁴, R⁵, and R⁶ are independently selected from H, OC₁₋₆alkyl, SC₁₋₆alkyl, C₁₋₄alkyl, CN, halogen, and haloC₁₋₄alkyl; preferably R², R³, R⁴, R⁵, and R⁶ are independently selected from H, OC₁₋₆alkyl, C₁₋₄alkyl, halogen, and haloC₁₋₄alkyl; in some embodiments, R², R³, R⁴, R⁵, and R⁶ are independently selected from H, C₁₋₄alkyl, halogen, and haloC₁₋₄alkyl;
R⁷ is selected from hydrogen, OC₁₋₆alkyl, SC₁₋₆alkyl, CN, OH, SH, halogen, NO₂, N(R^{m})₂, haloC₁₋₆alkyl; in some embodiments, R⁷ is selected from hydrogen, halogen, NO₂, haloC₁₋₆alkyl; in some embodiments, R⁷ is hydrogen;
Ar is phenyl, 5-6-membered heteroaryl or 5-6 membered heterocyclyl, wherein the phenyl, heteroaryl or heterocycyl is optionally substituted with one more substituents selected from OC₁₋₆alkyl, SC₁₋₆alkyl, CN, OH, SH, COOH, halogen, NO₂, N(R^{m})₂, C(O)OR^{m}, C(O)N(R^{m})₂, C(O)C₁₋₆alkyl, haloC₁₋₆alkyl, haloC₁₋₆alkyleneO, C₁₋₆alkyl, hydroxyC₁₋₆alkyl, dihydroxyC₁₋₁₀alkyl, C₃₋₆cycloalkyl; in some embodiments, the one more substituents selected from OC₁₋₆alkyl, CN, OH, COOH, halogen, NO₂, N(R^{m})₂, C(O)OR^{m}, C(O)N(R^{m})₂, C(O)C₁₋₆alkyl, haloC₁₋₆alkyl, haloC₁₋₆alkyleneO, C₁₋₆alkyl, hydroxyC₁₋₆alkyl, and C₃₋₆cycloalkyl; in some embodiment, Ar is phenyl, pyrindine, pyrimidine, piperidine, piperazine or 5-6 membered heterocyclyl, each of which is optionally substituted;
and
X is halogen or haloC₁₋₄alkyl; in some embodiments, X is halogen.

In some embodiments, the compound of Formula I is represented by Formula I-b,

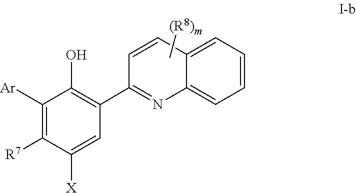

Wherein
R⁷ is selected from hydrogen, OC₁₋₆alkyl, SC₁₋₆alkyl, CN, OH, SH, halogen, NO₂, N(R^{m})₂, haloC₁₋₆alkyl; in some embodiments, R⁷ is selected from hydrogen, halogen, NO₂, haloC₁₋₆alkyl; in some embodiments, R⁷ is hydrogen;
R⁸ in each instance is independently selected from H, OC₁₋₆alkyl, SC₁₋₆alkyl, CN, OH, SH, COOH, halogen, NO₂, N(R^{m})₂, C(O)OR^{m}, C(O)N(R^{m})₂, C(O)C₁₋₆alkyl, haloC₁₋₆alkyl, haloC₁₋₆alkyleneO, C₁₋₆alkyl, hydroxyC₁₋₆alkyl, dihydroxyC₁₋₁₀alkyl, C₃₋₆cycloalkyl; in some embodiments, the one more R^{m} in each instance are selected from OC₁₋₆alkyl, CN, OH, COOH, halogen, NO₂, N(R^{m})₂, C(O)OR^{m}, C(O)N(R^{m})₂, C(O)C₁₋₆alkyl, haloC₁₋₆alkyl, haloC₁₋₆alkyleneO, C₁₋₆alkyl, hydroxyC₁₋₆alkyl, and C₃₋₆cycloalkyl; in some embodiments, R^{m} in each instance is are independently selected from H, OC₁₋₆alkyl, C₁₋₄alkyl, halogen, and haloC₁₋₄alkyl; in some embodiments, R^{m} in each instance is independently selected from H, C₁₋₄alkyl, halogen, and haloC₁₋₄alkyl;
m is 1, 2, 3, 4, 5, or 6;
Ar is phenyl, 5-6-membered heteroaryl or 5-6 membered heterocyclyl, wherein the phenyl, heteroaryl or heterocycyl is optionally substituted with one more substituents selected from OC₁₋₆alkyl, SC₁₋₆alkyl, CN, OH, SH, COOH, halogen, NO₂, N(R^{m})₂, C(O)OR^{m}, C(O)N(R^{m})₂, C(O)C₁₋₆alkyl, haloC₁₋₆alkyl, haloC₁₋₆alkyleneO, C₁₋₆alkyl, hydroxyC₁₋₆alkyl, dihydroxyC₁₋₁₀alkyl, C₃₋₆cycloalkyl; in some embodiments, the one more substituents selected from OC₁₋₆alkyl, CN, OH, COOH, halogen, NO₂, N(R^{m})₂, C(O)OR^{m}, C(O)N(R^{m})₂, C(O)C₁₋₆alkyl, haloC₁₋₆alkyl, haloC₁₋₆alkyleneO, C₁₋₆alkyl, hydroxyC₁₋₆alkyl, and C₃₋₆cycloalkyl; in some embodiment, Ar is phenyl, pyrindine, pyrimidine, piperidine, piperazine or 5-6 membered heterocyclyl, each of which is optionally substituted;
and
X is halogen or haloC₁₋₄alkyl; in some embodiments, X is halogen.

In some embodiments, the compound of Formula I is represented by Formula I-c,

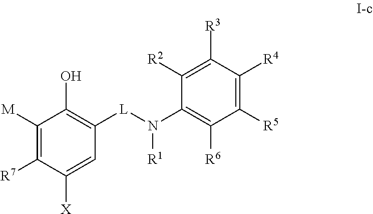

Wherein
M is OC₁₋₆alkyl, SC₁₋₆alkyl, C₁₋₄alkyl, CN, halogen, and haloC₁₋₄alkyl; in some embodiments, M is H;
L is C₁₋₃alkylene, SO2 or CO;
R¹ is hydrogen or C₁₋₆alkyl;
R², R³, R⁴, R⁵, and R⁶ are independently selected from H, OC₁₋₆alkyl, SC₁₋₆alkyl, C₁₋₄alkyl, CN, halogen, and haloC₁₋₄alkyl; preferably R², R³, R⁴, R⁵, and R⁶ are independently selected from H, OC₁₋₆alkyl, C₁₋₄alkyl, halogen, and haloC₁₋₄alkyl; in some embodiments, R², R³, R⁴, R⁵, and R⁶ are independently selected from H, C₁₋₄alkyl, halogen, and haloC₁₋₄alkyl;
R⁷ is selected from phenyl, 5-6-membered heteroaryl or 5-6 membered heterocyclyl, wherein the phenyl, heteroaryl or heterocycyl is optionally substituted with one more substituents selected from OC₁₋₆alkyl, SC₁₋₆alkyl, CN, OH, SH, COOH, halogen, NO₂, N(R^{m})₂, C(O)OR^{m}, C(O)N(R^{m})₂, C(O)C₁₋₆alkyl, haloC₁₋₆alkyl, haloC₁₋₆alkyleneO, C₁₋₆alkyl, hydroxyC₁₋₆alkyl, dihydroxyC₁₋₁₀alkyl, C₃₋₆cycloalkyl; in some embodiments, the one more substituents selected from OC₁₋₆alkyl, CN, OH, COOH, halogen, NO₂, N(R^{m})₂, C(O)OR^{m}, C(O)N(R^{m})₂, C(O)C₁₋₆alkyl, haloC₁₋₆alkyl, haloC₁₋₆alkyleneO, C₁₋₆alkyl, hydroxyC₁₋₆alkyl, and C₃₋₆cycloalkyl; in some embodiment, M is phenyl, pyrindine, pyrimidine, piperidine, piperazine or 5-6 membered heterocyclyl, each of which is optionally substituted;
and
X is halogen or haloC₁₋₄alkyl; in some embodiments, X is halogen.

In some embodiments, the compound of Formula I is represented by Formula I-d,

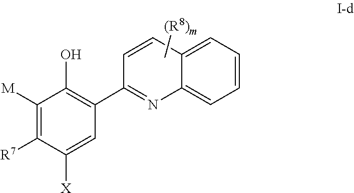

Wherein
M is OC₁₋₆alkyl, SC₁₋₆alkyl, C₁₋₄alkyl, CN, halogen, and haloC₁₋₄alkyl; in some embodiments, M is H;
R⁷ is selected from phenyl, 5-6-membered heteroaryl or 5-6 membered heterocyclyl, wherein the phenyl, heteroaryl or heterocycyl is optionally substituted with one more substituents selected from OC₁₋₆alkyl, SC₁₋₆alkyl, CN, OH, SH, COOH, halogen, NO₂, N(R^{m})₂, C(O)OR^{m}, C(O)N(R^{m})₂, C(O)C₁₋₆alkyl, haloC₁₋₆alkyl, haloC₁₋₆alkyleneO, C₁₋₆alkyl, hydroxyC₁₋₆alkyl, dihydroxyC₁₋₁₀alkyl, C₃₋₆cycloalkyl; in some embodiments, the one more substituents selected from OC₁₋₆alkyl, CN, OH, COOH, halogen, NO₂, N(R^{m})₂, C(O)OR^{m}, C(O)N(R^{m})₂, C(O)C₁₋₆alkyl, haloC₁₋₆alkyl, haloC₁₋₆alkyleneO, C₁₋₆alkyl, hydroxyC₁₋₆alkyl, and C₃₋₆cycloalkyl; in some embodiment, M is phenyl, pyrindine, pyrimidine, piperidine, piperazine or 5-6 membered heterocyclyl, each of which is optionally substituted;
R⁸ in each instance is independently selected from H, OC₁₋₆alkyl, SC₁₋₆alkyl, CN, OH, SH, COOH, halogen, NO₂, N(R^{m})₂, C(O)OR^{m}, C(O)N(R^{m})₂, C(O)C₁₋₆alkyl, haloC₁₋₆alkyl, haloC₁₋₆alkyleneO, C₁₋₆alkyl, hydroxyC₁₋₆alkyl, dihydroxyC₁₋₁₀alkyl, C₃₋₆cycloalkyl; in some embodiments, the one more R⁸ in each instance are selected from OC₁₋₆alkyl, CN, OH, COOH, halogen, NO₂, N(R^{m})₂, C(O)OR^{m}, C(O)N(R^{m})₂, C(O)C₁₋₆alkyl, haloC₁₋₆alkyl, haloC₁₋₆alkyleneO, C₁₋₆alkyl, hydroxyC₁₋₆alkyl, and C₃₋₆cycloalkyl; in some embodiments, R⁸ in each instance is are independently selected from H, OC₁₋₆alkyl, C₁₋₄alkyl, halogen, and haloC₁₋₄alkyl; in some embodiments, R⁸ in each instance is independently selected from H, C₁₋₄alkyl, halogen, and haloC₁₋₄alkyl;
m is 1, 2, 3, 4, 5, or 6;
and
X is halogen or haloC₁₋₄alkyl; in some embodiments, X is halogen.

In some embodiments, the compound is selected from

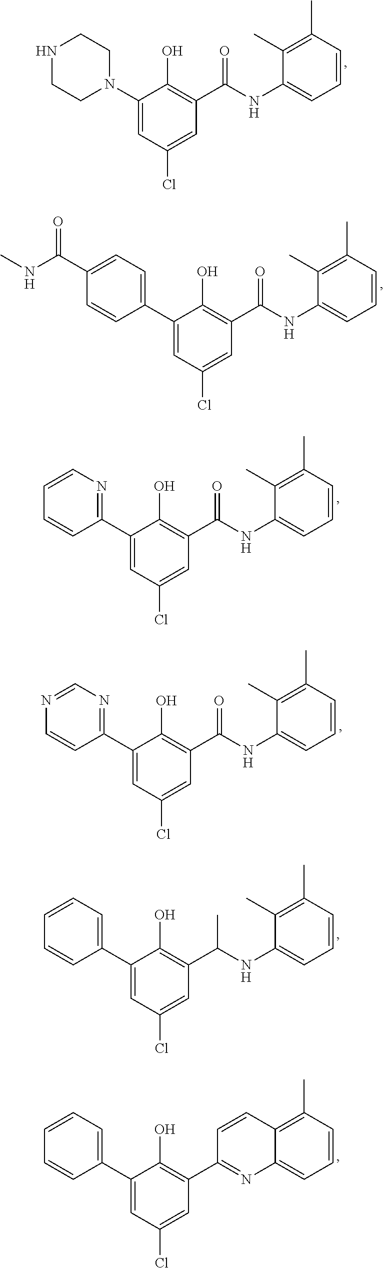 [SMILES: Cc1cccc(NC(=O)c2cc(Cl)cc(N3CCNCC3)c2O)c1C ; CNC(=O)c1ccc(-c2cc(Cl)cc(C(=O)Nc3cccc(C)c3C)c2O)cc1 ; Cc1cccc(NC(=O)c2cc(Cl)cc(-c3ccccn3)c2O)c1C ; Cc1cccc(NC(=O)c2cc(Cl)cc(-c3ccncn3)c2O)c1C ; Cc1cccc(NC(C)c2cc(Cl)cc(-c3ccccc3)c2O)c1C ; Cc1cccc2nc(-c3cc(Cl)cc(-c4ccccc4)c3O)ccc12]

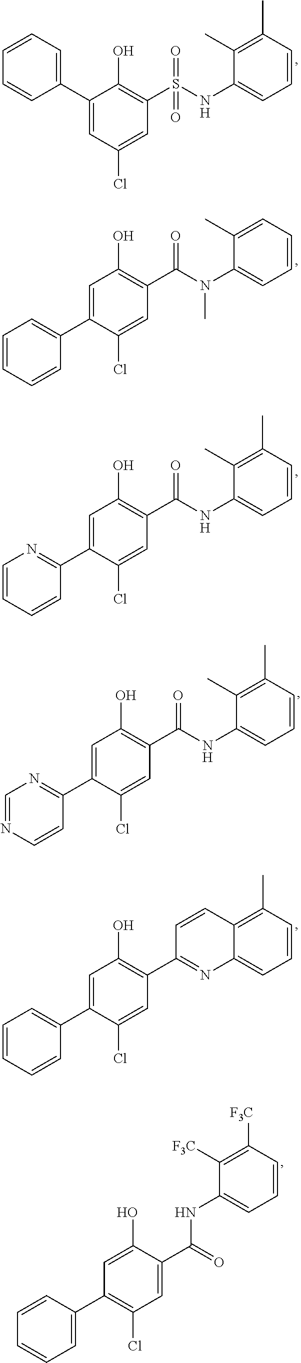 [SMILES: Cc1cccc(NS(=O)(=O)c2cc(Cl)cc(-c3ccccc3)c2O)c1C ; Cc1ccccc1N(C)C(=O)c1cc(Cl)c(-c2ccccc2)cc1O ; Cc1cccc(NC(=O)c2cc(Cl)c(-c3ccccn3)cc2O)c1C ; Cc1cccc(NC(=O)c2cc(Cl)c(-c3ccncn3)cc2O)c1C ; Cc1cccc2nc(-c3cc(Cl)c(-c4ccccc4)cc3O)ccc12 ; O=C(Nc1cccc(C(F)(F)F)c1C(F)(F)F)c1cc(Cl)c(-c2ccccc2)cc1O]

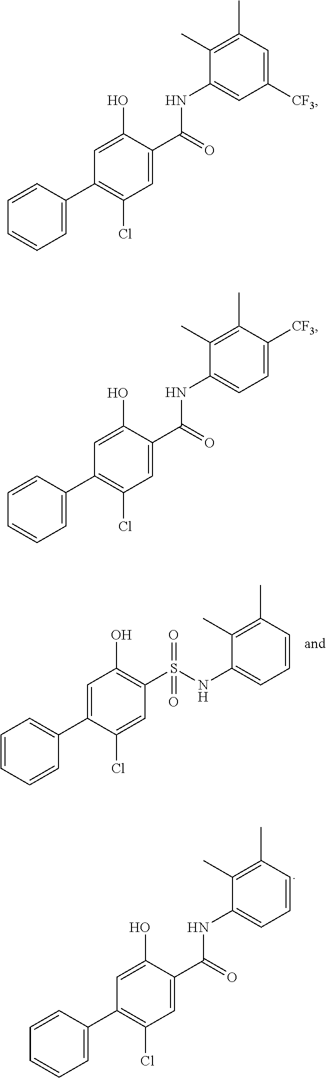 [SMILES: Cc1cc(C(F)(F)F)cc(NC(=O)c2cc(Cl)c(-c3ccccc3)cc2O)c1C ; Cc1c(NC(=O)c2cc(Cl)c(-c3ccccc3)cc2O)ccc(C(F)(F)F)c1C ; Cc1cccc(NS(=O)(=O)c2cc(Cl)c(-c3ccccc3)cc2O)c1C ; Cc1cccc(NC(=O)c2cc(Cl)c(-c3ccccc3)cc2O)c1C]

In some embodiments, the compound is in a salt form. In some embodiments, the compound is in a hydrochloride sate. Depending on the number of basic amine groups in the molecule, the compound may be a mono- or di-salt (e.g. di-hydrochloride chlorided salt).

Additional example compounds under Formula I are illustrated in the table below.

TABLE 1

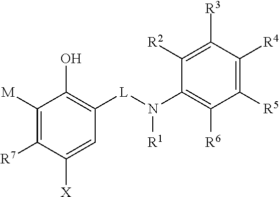

| | M | X | L | R¹ | R² | R³ | R⁴ | R⁵ | R⁶ | R⁷ |
|---|---|---|---|---|---|---|---|---|---|---|
| 1 | 4-F—Ph | Cl | C(O) | H | Me | Me | H | H | H | H |
| 2 | 4-NH₂—Ph | Cl | C(O) | H | Me | Me | H | H | H | H |
| 3 | 3-Me—Ph | Cl | C(O) | H | Me | Me | H | H | H | H |
| 3 | 4- | Cl | C(O) | H | Me | Me | H | H | H | H |
| | (MeOC(O))—Ph | | | | | | | | | |
| 5 | 4-F—Ph | F | C(O) | H | Me | Me | H | H | H | H |
| 6 | 4-F—Ph | Cl | CHCH₃ | H | Me | Me | H | H | H | H |
| 7 | 4-F—Ph | Cl | CHCH₃ | Me | Me | Me | H | H | H | H |
| 8 | 4-F—Ph | Cl | CHCH₃ | H | H | Me | H | H | H | H |
| 9 | 4-F—Ph | Cl | C(O) | H | Me | Me | F | H | H | H |
| 10 | 4-F—Ph | Cl | C(O) | H | Me | Me | H | F | H | H |
| 11 | 4-F—Ph | Cl | C(O) | H | Me | Me | H | H | F | H |
| 12 | 4-F—Ph | Cl | C(O) | H | Me | Me | H | H | H | F |
| 13 | 4-F—Ph | Cl | C(O) | H | Me | Me | H | H | H | Me |
| 14 | 4-F-pyridyl | Cl | C(O) | H | Me | Me | H | H | H | H |
| 15 | 4-Me- | Cl | C(O) | H | Me | Me | H | H | Cl | H |
| | pyridyl | | | | | | | | | |
| 16 | 2-Me- | Cl | C(O) | H | Me | Me | H | Cl | H | H |
| | pyrimidyl | | | | | | | | | |
| 17 | 2-thiophenyl | Cl | C(O) | H | Me | Me | H | H | H | H |
| 18 | 4- | Cl | C(O) | H | Me | Me | H | H | H | H |
| | morpholinyl | | | | | | | | | |
| 19 | 1- | Cl | C(O) | H | Me | Me | H | H | H | H |
| | piperidinyl | | | | | | | | | |
| 20 | 4-methyl- | Cl | C(O) | H | Me | Me | H | H | H | H |
| | piperazinyl | | | | | | | | | |
| 21 | 1- | Cl | C(O) | H | Me | Me | H | H | H | H |
| | pyrrolidinyl | | | | | | | | | |
| 22 | 1-methyl-2- | Cl | CHCH₃ | H | CF₃ | Me | H | H | H | H |
| | pyrrolidinyl | | | | | | | | | |
| 23 | 2-(4-Me- | Cl | SO₂ | Me | CF₃ | CF₃ | Me | H | H | Et |
| | pyridinyl) | | | | | | | | | |
| 24 | 2-F-phenyl | Cl | SO₂ | Me | Et | CF₃ | Me | H | H | Et |
| 25 | H | F | C(O) | Me | Et | CF₃ | H | H | H | 4-F—Ph |
| 26 | H | Cl | C(O) | H | CF₃ | Me | H | H | H | 4-NH₂—Ph |
| 27 | Me | Cl | C(O) | H | CF₃ | CF₃ | H | H | H | 3-Me—Ph |
| 28 | OMe | Cl | CHCH₃ | H | Et | CF₃ | H | H | H | 4- |
| | | | | | | | | | | (MeOC(O))—Ph |
| 29 | F | Cl | SO₂ | Me | CF₃ | Me | H | H | H | 4-F—Ph |
| 30 | CF₃ | Cl | SO₂ | H | H | Me | CF₃ | H | H | 4-F-pyridyl |
| 31 | H | Cl | C(O) | H | H | CF₃ | CF₃ | H | H | 4-Me- |
| | | | | | | | | | | pyridyl |
| 32 | H | F | C(O) | H | H | Me | CF₃ | H | H | 2-Me- |
| | | | | | | | | | | pyrimidyl |
| 33 | H | Cl | C(O) | H | CF₃ | Me | H | H | CF₃ | 4-NH₂—Ph |
| 34 | H | Cl | C(O) | H | CF₃ | Me | H | CF₃ | H | 4-NH₂—Ph |
| 35 | Ph | Cl | C(O) | H | Me | Me | H | Cl | H | H |
| 36 | Ph | Cl | C(O) | H | Me | Me | Cl | H | H | H |
| 37 | Ph | Cl | C(O) | H | Me | Me | H | F | H | H |
| 38 | Ph | Cl | C(O) | H | Me | Me | H | Br | H | H |
| 39 | H | Cl | C(O) | H | Me | Me | H | Cl | H | Ph |
| 40 | H | Cl | C(O) | H | Me | Me | H | F | H | Ph |
| 41 | H | Cl | C(O) | H | Me | Me | H | Br | H | Ph |
| 42 | H | Cl | C(O) | H | Me | Me | Cl | H | H | Ph |

Additional examples of I-b are as shown below.

TABLE 2

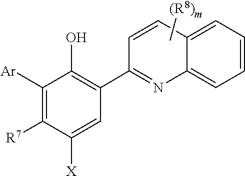

| | Ar | R⁷ | X | 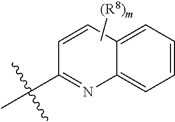 |
|---|---|---|---|---|
| 43 | 4-F—Ph | H | Cl | 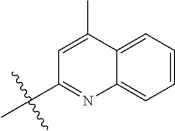 [SMILES: *c1cc(C)c2ccccc2n1] |
| 44 | 4-NH₂—Ph | H | F | 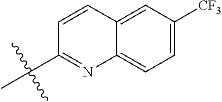 [SMILES: *c1ccc2cc(C(F)(F)F)ccc2n1] |
| 45 | 3-Me—Ph | H | Cl | 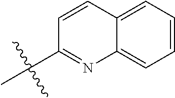 [SMILES: *c1ccc2ccccc2n1] |
| 46 | 4- (MeOC(O))—Ph | Me | Cl | 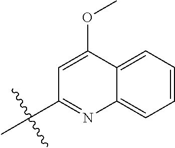 [SMILES: *c1cc(OC)c2ccccc2n1] |
| 47 | 4-F—Ph | F | Cl | 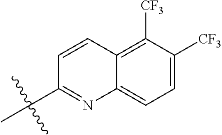 [SMILES: *c1ccc2c(C(F)(F)F)c(C(F)(F)F)ccc2n1] |
| 48 | 4-F—Ph | CF3 | Cl | 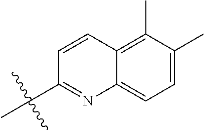 [SMILES: *c1ccc2c(C)c(C)ccc2n1] |
| 49 | 4-F—Ph | OMe | Cl | 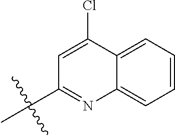 [SMILES: *c1cc(Cl)c2ccccc2n1] |
| 50 | Ph | H | Cl | 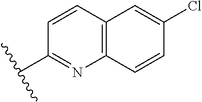 [SMILES: *c1ccc2cc(Cl)ccc2n1] |

Additional examples of I-d are as shown below.

TABLE 3
I-d

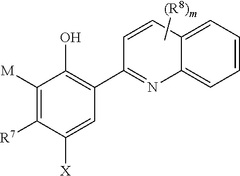

| | R⁷ | M | X | 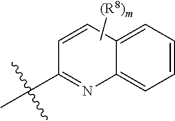 |
|---|---|---|---|---|
| 51 | 4-F—Ph | H | Cl | 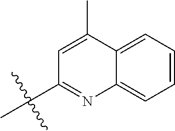 [SMILES: *c1cc(C)c2ccccc2n1] |
| 52 | 4-NH₂—Ph | H | F | 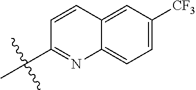 [SMILES: *c1ccc2cc(C(F)(F)F)ccc2n1] |
| 53 | 3-Me—Ph | H | Cl | 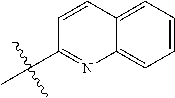 [SMILES: *c1ccc2ccccc2n1] |
| 54 | 4- (MeOC(O))—Ph | Me | Cl | 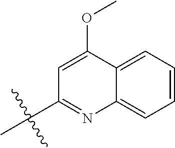 [SMILES: *c1cc(OC)c2ccccc2n1] |
| 55 | 4-F—Ph | F | Cl | 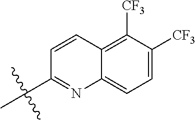 [SMILES: *c1ccc2c(C(F)(F)F)c(C(F)(F)F)ccc2n1] |
| 56 | 4-F—Ph | CF3 | Cl | 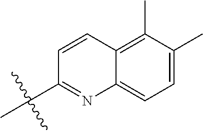 [SMILES: *c1ccc2c(C)c(C)ccc2n1] |
| 57 | 4-F—Ph | OMe | Cl | 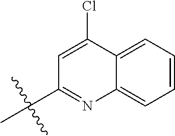 [SMILES: *c1cc(Cl)c2ccccc2n1] |
| 58 | Ph | H | Cl | 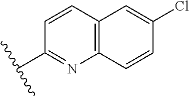 [SMILES: *c1ccc2cc(Cl)ccc2n1] |

Also provided herein is a compound of Formula II or a pharmaceutically acceptable salt thereof.

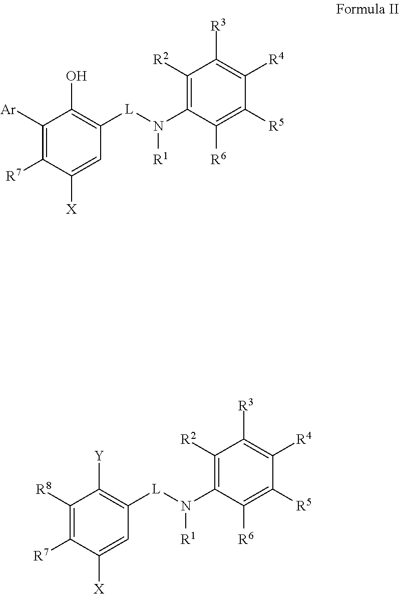

X is halogen;
Y is C(O)OR^{m}, C(O)N(R^{m})₂, C(O)C₁₋₆alkyl,
L is C₁₋₃alkylene optionally substituted with an oxo (═O);
R¹ is hydrogen, C₁₋₆alkyl or C(O)C₁₋₄alkyl,
R² and R³ are independently selected from the group consisting of OC₁₋₆alkyl, SC₁₋₆alkyl, C₁₋₄alkyl, CN, halogen, C₁₋₆alkylene-CN, OC₁₋₆alkylene-CN, haloC₁₋₆alkyl, SC₁₋₆alkylene-CN, C₂₋₆alkynyl, C₂₋₆alkenyl, and C₁₋₆alkylSO₂ (sulfone);
R⁴, R⁵, R⁶, R⁷ and R⁸ are independently selected from the group consisting of hydrogen, deuterium, OC₁₋₆alkyl, SC₁₋₆alkyl, CN, OH, SH, halogen, NO₂, N(R^{m})₂, C(O)OR^{m}, C(O)N(R^{m})₂, C(O)C₁₋₆alkyl, haloC₁₋₆alkyl, haloC₁₋₆alkyleneO, C₁₋₆alkyl, hydroxyC₁₋₆alkyl, dihydroxyC₁₋₁₀alkyl, C₃₋₆cycloalkyl, C(═NC₁₋₆alkyl)C₁₋₆alkyl, OC(O)N(R^{m})₂, C(O)SR^{m}, OC₁₋₆alkyleneOC₁₋₆alkyl, OC₁₋₆alkyleneO-haloC₁₋₆alkyl, SC₁₋₆alkyleneOC₁₋₆alkyl, SC₁₋₆alkyleneSC₁₋₆alkyl, OC₁₋₆ alkyleneSC₁₋₆alkyl, SC₁₋₆alkyleneO-haloC₁₋₆alkyl, SC₁₋₆alkyleneS-haloC₁₋₆alkyl, OC₁₋₆ alkyleneS-haloC₁₋₆alkyl, C₁₋₆alkylene-CN, OC₁₋₆alkylene-CN, SC₁₋₆alkylene-CN, OC₁₋₆ alkylene-N(R^{m})₂, C₂₋₆alkynyl, C₂₋₆alkenyl, SO₂N(R^{m})₂, NR^{m}SO₂C₁₋₆alkyl, C₁₋₆alkylSO₂ (sulfone), S(O)OH, C₁₋₆alkylS(O) (sulfoxide), nitroso, C₁₋₆alkylOSO₂; and
R^{m} in each instance is independently hydrogen or C₁₋₆alkyl or halo-C₁₋₆alkyl.

In some embodiments, Y is COOH or C(O)OC₁₋₆alkyl.

In some embodiments, X is fluorine or chlorine. In some embodiments, X is chlorine.

In some embodiments, R¹ is hydrogen or methyl. In some embodiments, R² and R³ are independently selected from the group consisting of C₁₋₄alkyl, CN, and halogen. In some embodiments, R² and R³ are independently C₁₋₃alkyl. R⁴, R⁵, and R⁶ are independently selected from the group consisting of hydrogen, OH, SH, halogen, NO₂, N(R^{m})₂, C(O)OR^{m}, and C(O)N(R^{m})₂. In some embodiments, R⁴ and R⁶ are each hydrogen and R⁵ is hydrogen or COOH. In some embodiments, R⁷ is hydrogen or C₁₋₃alkyl.

In some embodiments, L is C(O). In some embodiments, R¹ is H.

In some embodiments, L is CH₂, CHCH₃ or CHCH₂CH₃. In some embodiments, R¹ is H.

In some embodiments, the compound is selected from

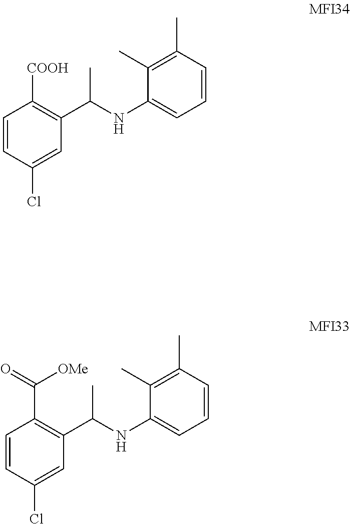 [SMILES: Cc1cccc(NC(C)c2cc(Cl)ccc2C(=O)O)c1C ; COC(=O)c1ccc(Cl)cc1C(C)Nc1cccc(C)c1C]

Also disclosed herein is a compound of Formula III or a pharmaceutically acceptable salt thereof.

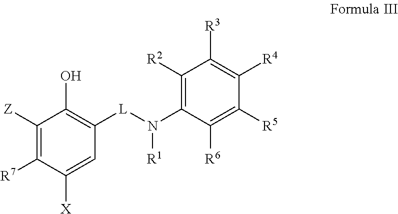

wherein:
X is halogen;
Z is selected from the group consisting of C(O)OR^{m}, C(O)N(R^{m})₂, C(O)C₁₋₆alkyl, OC(O)N(R^{m})₂, OC(O)C₁₋₆alkyl, N(R^{m})C(O)C₁₋₆alkyl, N(R^{m})C(O)C₂₋₆alkenyl, C(O)C₁₋₆alkyl, C₁₋₆alkyleneC(O)OR^{m}, C₁₋₆alkyleneC(O)N(R^{m})₂, C₁₋₆alkyleneC(O)C₁₋₆alkyl, C₁₋₆alkyleneOC(O)N(R^{m})₂, C₁₋₆alkyleneOC(O)C₁₋₆alkyl, C₁₋₆alkyleneC₁₋₆alkyleneN(R^{m})C(O)C₁₋₆alkyl, C₁₋₆alkyleneN(R^{m})C(O)C₂₋₆alkenyl, and C₁₋₆alkyleneC(O)C₁₋₆alkyl,
L is C₁₋₃alkylene optionally substituted with an oxo (═O);
R¹ is hydrogen, C₁₋₆alkyl or C(O)C₁₋₄alkyl,
R² and R³ are independently selected from the group consisting of OC₁₋₆alkyl, SC₁₋₆alkyl, C₁₋₄alkyl, CN, halogen, C₁₋₆alkylene-CN, OC₁₋₆alkylene-CN, haloC₁₋₆alkyl, SC₁₋₆alkylene-CN, C₂₋₆alkynyl, C₂₋₆alkenyl, and C₁₋₆alkylSO₂ (sulfone);
R⁴, R⁵, R⁶, and R⁷ are independently selected from the group consisting of hydrogen, deuterium, OC₁₋₆alkyl, SC₁₋₆alkyl, CN, OH, SH, halogen, NO₂, N(R^{m})₂, C(O)OR^{m}, C(O)N(R^{m})₂, C(O)C₁₋₆alkyl, haloC₁₋₆alkyl, haloC₁₋₆alkyleneO, C₁₋₆alkyl, hydroxyC₁₋₆alkyl, dihydroxyC₁₋₁₀alkyl, C₃₋₆cycloalkyl, C(═NC₁₋₆alkyl)C₁₋₆alkyl, OC(O)N(R^{m})₂, C(O)SR^{m}, OC₁₋₆alkyleneOC₁₋₆alkyl, OC₁₋₆ alkyleneO-haloC₁₋₆alkyl, SC₁₋₆alkyleneOC₁₋₆alkyl, SC₁₋₆alkyleneSC₁₋₆alkyl, OC₁₋₆alkyleneSC₁₋₆alkyl, SC₁₋₆alkyleneO-haloC₁₋₆alkyl, SC₁₋₆alkyleneS-haloC₁₋₆alkyl, OC₁₋₆alkyleneS-haloC₁₋₆alkyl, C₁₋₆alkylene-CN, OC₁₋₆alkylene-CN, SC₁₋₆alkylene-CN, OC₁₋₆alkylene-N(R^{m})₂, C₂₋₆alkynyl, C₂₋₆alkenyl, SO₂N(R^{m})₂, NR^{m}SO₂C₁₋₆alkyl, C₁₋₆alkylSO₂ (sulfone), S(O)OH, C₁₋₆alkylS(O) (sulfoxide), nitroso, C₁₋₆alkylOSO₂; and
R^{m} in each instance is independently hydrogen or C₁₋₆alkyl or halo-C₁₋₆alkyl.

In some embodiments, Z is selected from C₁₋₆alkyleneC(O)OR^{m}, C₁₋₆alkyleneC(O)N(R^{m})₂, C₁₋₆alkyleneC(O)C₁₋₆alkyl, C₁₋₆alkyleneOC(O)N(R^{m})₂, C₁₋₆alkyleneOC(O)C₁₋₆alkyl, C₁₋₆alkyleneC₁₋₆alkyleneN(R^{m})C(O)C₁₋₆alkyl, C₁₋₆alkyleneN(R^{m})C(O)C₂₋₆alkenyl, and C₁₋₆alkyleneC(O)C₁₋₆alkyl. In some embodiments, Z is C₁₋₆alkyleneOC(O)C₁₋₆alkyl, or C₁₋₆alkyleneC₁₋₆alkyleneN(R^{m})C(O)C₁₋₆alkyl, or C₁₋₆alkyleneN(R^{m})C(O)C₂₋₆alkenyl. In some embodiments, Z is C₁₋₆alkyleneC₁₋₆alkyleneN(R^{m})C(O)C₁₋₆alkyl, or C₁₋₆alkyleneN(R^{m})C(O)C₂₋₆alkenyl. In some embodiments, Z is C₁₋₆alkyleneN(R^{m})C(O)C₂₋₆alkenyl. In some embodiments, Z is C₂₋₄alkyleneN(R^{m})C(O)C₂₋₆alkenyl. In some embodiments, Z is C₂₋₄alkyleneN(R^{m})C(O)CHCH₂. In some embodiments, R^{m} is H.

In some embodiments, X is fluorine or chlorine. In some embodiments, X is chlorine.

In some embodiments, R¹ is hydrogen or methyl. In some embodiments, R² and R³ are independently selected from the group consisting of C₁₋₄alkyl, CN, and halogen. In some embodiments, R² and R³ are independently C₁₋₃alkyl. R⁴, R⁵, and R⁶ are independently selected from the group consisting of hydrogen, OH, SH, halogen, NO₂, N(R^{m})₂, C(O)OR^{m}, and C(O)N(R^{m})₂. In some embodiments, R⁴ and R⁶ are each hydrogen and R⁵ is hydrogen or COOH. In some embodiments, R⁷ is hydrogen or C₁₋₃alkyl.

In some embodiments, L is C(O). In some embodiments, R¹ is H.

In some embodiments, L is CH₂, CHCH₃ or CHCH₂CH₃. In some embodiments, R¹ is H.

In some embodiments, the compound is

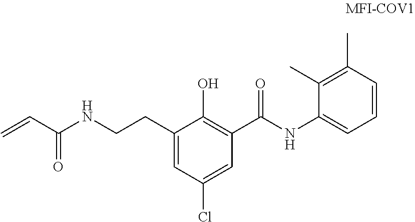 [SMILES: C=CC(=O)NCCc1cc(Cl)cc(C(=O)Nc2cccc(C)c2C)c1O]

In some embodiments, the compound disclosed herein is not one of the following:

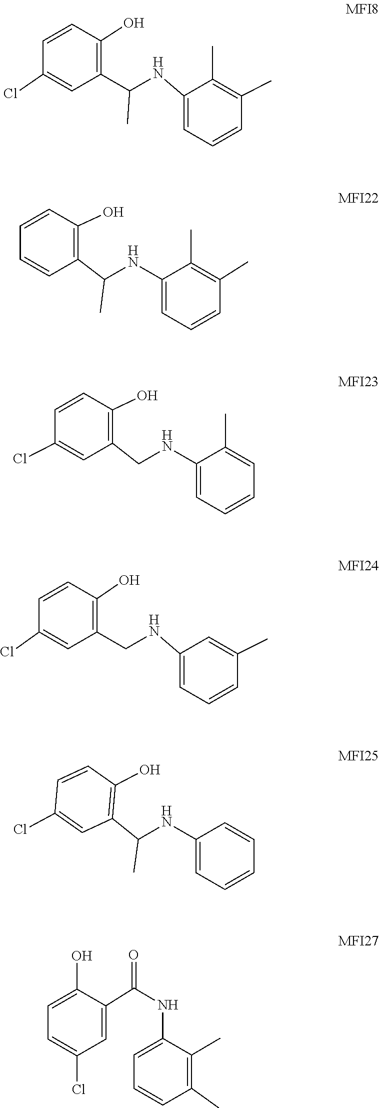 [SMILES: Cc1cccc(NC(C)c2cc(Cl)ccc2O)c1C ; Cc1cccc(NC(C)c2ccccc2O)c1C ; Cc1ccccc1NCc1cc(Cl)ccc1O ; Cc1cccc(NCc2cc(Cl)ccc2O)c1 ; CC(Nc1ccccc1)c1cc(Cl)ccc1O ; Cc1cccc(NC(=O)c2cc(Cl)ccc2O)c1C]

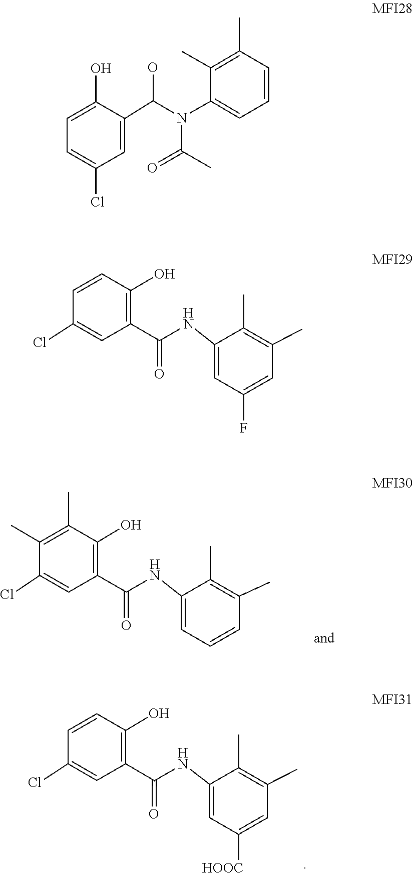

### Pharmaceutical Composition and Kit

Another aspect of the patent specification provides a pharmaceutical composition comprising a compound of Formula I or a pharmaceutically acceptable salt thereof disclosed herein and a pharmaceutically acceptable carrier, excipient, or diluent. Compounds described in this patent specification may be formulated by any method well known in the art and may be prepared for administration by any route, including, without limitation, parenteral, peroral, sublingual, buccal, intrathecal, transdermal, topical, subcutaneous, intramuscular, intraperitoneal, intranasal, intratracheal, or intrarectal.

Nonlimiting examples of pharmaceutically acceptable carriers include physiologically acceptable surface active agents, glidants, plasticizers, diluents, excipients, smoothing agents, suspension agents, complexing agents, film forming substances, and coating assistants. Preservatives, stabilizers, dyes, sweeteners, fragrances, flavoring agents, and the like may be provided in the pharmaceutical composition. For example, sodium benzoate, ascorbic acid and esters of p-hydroxybenzoic acid may be added as preservatives. In addition, antioxidants and suspending agents may be used. In various embodiments, alcohols, esters, sulfated aliphatic alcohols, and the like may be used as surface active agents. Suitable exemplary binders include crystalline cellulose, sucrose, D-mannitol, dextrin, hydroxypropylcellulose, hydroxypropylmethylcellulose, polyvinylpyrrolidone, and the like. Suitable exemplary disintegrants include starch, carboxymethylcellulose, calcium carboxymethylcellulose, croscarmellose sodium, sodium carboxymethylstarch, and the like. Suitable exemplary solvents or dispersion media include water, alcohol (for example, ethanol), polyols (for example, glycerol, propylene glycol, and polyethylene glycol, sesame oil, corn oil, and the like), and suitable mixtures thereof that are physiologically compatible. Suitable exemplary solubilizing agents include polyethylene glycol, propylene glycol, D-mannitol, benzylbenzoate, cyclodextrins, ethanol, trisaminomethane, cholesterol, triethanolamine, sodium carbonate, sodium citrate, and the like. Suitable exemplary suspending agents include surfactants such as stearyltriethanolamine, sodium laurylsulfate, laurylaminopropionic acid, lecithin, benzalkonium chloride, benzethonium chloride, glycerin monostearate, coconut oil, olive oil, sesame oil, peanut oil, soya and the like; and hydrophilic polymers such as polyvinyl alcohol, polyvinylpyrrolidone, sodium carboxymethylcellulose, methylcellulose, hydroxymethylcellulose, hydroxyethylcellulose, hydroxypropylcellulose, and the like. Suitable exemplary isotonic agent includes sodium chloride, glycerin, D-mannose, and the like. Suitable exemplary buffer agents include buffer solutions of salts, such as phosphate, acetates, carbonates, and citrates. Suitable exemplary soothing agents include benzyl alcohol, and the like. Suitable exemplary antiseptic substances include para-oxybenzoic acid esters, benzethonium chloride, benzalkonium chloride, chlorobutanol, benzyl alcohol, phenethyl alcohol, dehydroacetic acid, sorbic acid, and the like. Suitable exemplary antioxidants include sulfite salts, ascorbic acid, and the like. Suitable exemplary sealers include, but are not limited to HPMC (or hypromellose), HPC, PEG and combinations thereof. Suitable exemplary lubricants include magnesium stearate, calcium stearate, talc, colloidal silica, hardened oil and the like.

In further exemplary embodiments for solid preparations, carriers or excipients include diluents, lubricants, binders, and disintegrants. In exemplary embodiments for liquid preparations, carriers include solvents, solubilizing agents, suspending agents, isotonic agents, buffer agents, soothing agents, and the like. Acceptable additional carriers or diluents for therapeutic use and the general procedures for the preparation of pharmaceutical compositions are well known in the pharmaceutical art, and are described, for example, in Remington's Pharmaceutical Sciences, 18th Ed., Mack Publishing Co., Easton, PA (1990), which is incorporated herein by reference in its entirety.

The compound of Formula I may also be in a pharmaceutically acceptable salt form. Examples of such salts include, but are not limited to acid addition salts formed with inorganic acids (for example, hydrochloric acid, hydrobromic acid, sulfuric acid, phosphoric acid, nitric acid, and the like), and salts formed with organic acids such as acetic acid, oxalic acid, tartaric acid, succinic acid, malic acid, ascorbic acid, benzoic acid, tannic acid, pamoic acid, alginic acid, polyglutamic acid, naphthalenesulfonic acid, naphthalenedisulfonic acid, and polygalacturonic acid. The compounds can also be administered as pharmaceutically acceptable quaternary salts known by those skilled in the art, which specifically include the quaternary ammonium salt, wherein the counterion includes, for example, chloride, bromide, iodide, —O-alkyl, toluenesulfonate, methylsulfonate, sulfonate, phosphate, or carboxylate (such as benzoate, succinate, acetate, glycolate, maleate, malate, citrate, tartrate, ascorbate, benzoate, cinnamoate, mandeloate, benzyloate, and diphenylacetate).

A related aspect provides a kit, which includes a compound of Formula I or a pharmaceutically acceptable salt thereof, or a pharmaceutical composition thereof and an instruction for treating or preventing certain diseases or conditions. In some embodiments, the kit further includes an additional secondary therapeutic agent.

In some embodiments, the secondary agent is an anti-cancer agent. In any of the methods or kit described herein, the administration or inclusion of a secondary agent having a cytotoxic effect on a cancer cell is contemplated. A cytotoxic effect refers to the depletion, elimination and/or the killing of target cells (i.e., tumor cells). The cytotoxic agent may be at least one selected from the group consisting of an antimetabolite, a mitotic inhibitor, an alkylating agent, an antibody-based EGFR inhibitor, an antibody based HER2/3 inhibitor, an angiogenesis inhibitor, a mTOR inhibitor, a CDK4 and CDK6 inhibitor or an aromatase inhibitor. The combination may include at least two cytotoxic agents. For example, the combination may include at least 2, at least 3, or at least 4 selected from the group consisting of an antimetabolite, a mitotic inhibitor, an alkylating agent, an angiogenesis inhibitor, or all of them.

The antimetabolite may be a drug that inhibits DNA synthesis in cells by suppressing formation of purines or pyrimidines, which are bases of a nucleotide. In one embodiment, the antimetabolite may be selected from the group consisting of Capecitabine, 5-Fluorouracil, Gemcitabine, Pemetrexed, Methotrexate, 6-Mercaptopurine, Cladribine, Cytarabine, Doxifludine, Floxuridine, Fludarabine, Hydroxycarbamide, decarbazine, hydroxyurea, and asparaginase. In a more specific embodiment, the antimetabolite is a base analog, with the term base analogs herein including nucleotide and nucleoside analogs in addition to purine base analogs such as 5-fluorouracil.

The mitotic inhibitor may be a microtubule-destabilizing agent, a microtubule-stabilizing agent, or a combination thereof. The mitotic inhibitor may be selected from taxanes, vinca alkaloids, epothilone, or a combination thereof. In a specific embodiment, the mitotic inhibitor is a taxane, for example including but not limited to, paclitaxel, docetaxel and cabazitaxel. In another specific embodiment, the mitotic inhibitor is a vinca alkaloid or its derivative, for example including but not limited to, vinblastine, vincristine, vinflunine, vinorelbine, vincaminol, vinburnine, vineridine and vindesine.

The mitotic inhibitor may be selected from BT-062, HMN-214, eribulin mesylate, vindesine, EC-1069, EC-1456, EC-531, vintafolide, 2-methoxyestradiol, GTx-230, trastuzumab emtansine (T-DM1), crolibulin, D1302A-maytansinoid conjugates IMGN-529, lorvotuzumab mertansine, SAR-3419, SAR-566658, IMP-03138, topotecan/vincristine combinations, BPH-8, fosbretabulin tromethamine, estramustine phosphate sodium, vincristine, vinflunine, vinorelbine, RX-21101, cabazitaxel, STA-9584, vinblastine, epothilone A, patupilone, ixabepilone, Epothilone D, paclitaxel, docetaxel, DJ-927, discodermolide, eleutherobin, and pharmaceutically acceptable salts thereof or combinations thereof.

Non-limiting examples of checkpoint inhibitors include those that target PD-1, PD-L1, CTLA4 and TIGIT (T cell immunoglobulin and ITIM domain). Further examples include Ipilimumab (Yervoy®; blocking a checkpoint protein called CTLA-4); pembrolizumab (Keytruda®), Cemiplimab (Libtayo) and nivolumab (Opdivo®) (targeting another checkpoint protein called PD-1); atezolizumab (Tecentriq®), Avelumab (Bavencio), and Durvalumab (Imfinzi) (targeting PD-L1); MK-7684, Etigilimab/OMP-313 M32, Tiragolumab/MTIG7192A/RG-6058, BMS-986207, AB-154 and ASP-8374 (targeting TIGIT), and V-domain Ig suppressor of T cell activation (VISTA).

The EGFR inhibitors may be selected from erlotinib, gefitinib, lapatinib, canetinib, pelitinib, neratinib, (R,E)-N-(7-chloro-1-(1-(4-(dimethylamino)but-2-enoyl)azepan-3-yl)-1H-benzo[d]imidazol-2-yl)-2-methylisonicotinamide, Trastuzumab, Margetuximab, panitumumab, matuzumab, necitumumab, pertuzumab, nimotuzumab, zalutumumab, cetuximab, icotinib, afatinib, and pharmaceutically acceptable salt thereof. In one embodiment the EGFR inhibitor may be an antibody based EGFR inhibitor such as cetuximab and in another embodiment, it is necitumumab and yet in another embodiment it is panitumumab. The molecularly targeted agent may be an anti-EGFR family antibody or a complex including the anti-EGFR family antibody. The anti-EGFR family antibody may be an anti-HER1 antibody, an anti-HER2 antibody, or an anti-HER4 antibody.

Further examples of agents for chemotherapy include SHP2 inhibitors (e.g. RMC-4550 and RMC-4630), phosphatase inhibitors (e.g. Tautomycin), CDK 4/6 inhibitors (abemaciclib (Lilly), palbociclib (Pfizer)), protein-protein interaction disruptors (BI 1701963), HSP90 inhibitor, tubulin inhibitor, apoptosis inhibitor, chemopreventative agent, and therapies targeting PBK/AKT/mTOR pathway.

Antibody-drug conjugates have emerged as a breakthrough approach to the development of cancer therapeutics. Cancer is one of the leading causes of deaths in the world. Antibody-drug conjugates (ADCs) comprise monoclonal antibodies (MAbs) that are covalently linked to cell-killing drugs. This approach combines the high specificity of MAbs against their antigen targets with highly potent cytotoxic drugs, resulting in “armed” MAbs that deliver the payload (drug) to tumor cells with enriched levels of the antigen. Targeted delivery of the drug also minimizes its exposure in normal tissues, resulting in decreased toxicity and improved therapeutic index. The approval of two ADC drugs, ADCETRIS® (brentuximab vedotin) in 2011 and KADCYLA® (trastuzumab emtansine or T-DM1) in 2013 by FDA validated the approach. Another example is Trastuzumab duocarmazine. There are currently more than 30 ADC drug candidates in various stages of clinical trials for cancer treatment. As antibody engineering and linker-payload optimization are becoming more and more mature, the discovery and development of new ADCs are increasingly dependent on the identification and validation of new targets that are suitable to this approach and the generation of targeting MAbs. Two criteria for ADC targets are upregulated/high levels of expression in tumor cells and robust internalization.

### Method of Treating Diseases

Another aspect of the patent specification provides for methods for treating a disease or condition associated with imbalance between mitochondrial fission and fusion, including for example abnormal or elevated mitochondrial fusion and abnormal or decreased mitochondrial fission. The method includes administering to a subject in need thereof the compound of formula (I), a pharmaceutically acceptable salt thereof, or a corresponding pharmaceutical composition disclosed herein.

The compounds of this patent specification directly target mitofusins and impact on mitochondrial dynamics. Diseases or conditions treatable with the compounds and their pharmaceutically acceptable salts include for example neurodegenerative diseases, metabolic disease, cardiovascular diseases, autoimmune disease, hypertension, inflammatory disease, ageing and cancer.

In some embodiments, the disease treatable with the methods disclosed herein is cancer including for example breast cancer, colorectal cancer, gastric cancer, glioma, anal cancer, appendix cancer, bile duct cancer (i.e., cholangiocarcinoma), bladder cancer, brain tumor, cervical cancer, esophageal cancer, eye cancer, fallopian tube cancer, kidney cancer, liver cancer, lung cancer, medulloblastoma, melanoma, oral cancer, ovarian cancer, pancreatic cancer, parathyroid disease, penile cancer, pituitary tumor, prostate cancer, rectal cancer, skin cancer, gastric cancer, testicular cancer, throat cancer, thyroid cancer, uterine cancer, vaginal cancer, vulvar cancer, leukemia, lymphoma or a solid tumor, acute lymphoblastic leukemia (ALL), acute myeloid leukemia (AML), chronic lymphocytic leukemia (CLL) or chronic myeloid leukemia (CMIL), non-Hodgkin's lymphoma, myeloma, multiple myeloma, prostate cancer, skin cancer, colon cancer, brain cancer, head-neck cancer, glioma, glioblastoma, stomach cancer, cancer of the gastrointestinal tract, non-small cell lung cancer (NSCLC), small-cell lung carcinoma, adrenal carcinoma, renal cell carcinoma, soft-tissue sarcoma, rhabdomyosarcoma, Wilms' tumor.

Although inhibition of mitochondrial fusion induces minority MOMP, sub-lethal activation of caspases-3/7 and DNA damage as downstream events of minority MOMP, it does not induce cell death. Such insight provides a new opportunity to use inhibition of MFN1/2 activity to sensitize cells to other agents promoting apoptosis. As demonstrated in the example section, BV6, a SMAC mimetic that operates downstream of MOMP, when combined with MFI8 can synergistically induces cell death. Similar combinatorial strategies can have broad applications in cancer therapy by enhancing the activity of pro-apoptotic drugs to induce caspase-mediated apoptosis or evade resistance mechanisms. Furthermore, inhibition of mitochondrial fusion may alter the transcription of several genes that are involved in DNA repair and respiration, suggesting that changes in mitochondrial shape can impact gene transcription of diverse gene sets. These are possible homeostatic mechanisms that cells use to counteract mitochondrial stress.

In some embodiments, the disease treatable with the methods disclosed herein is a cardiovascular disease including for example arrhythmia, ischemic heart disease, hypertensive heart disease and pulmonary hypertensive heart disease, valvular disease, congenital heart disease, cardiomyopathy, stroke ischemic heart disease, cardiac ischemia-reperfusion injury, myocardial infarction, chemotherapy-induced cardiotoxicity, arteriosclerosis, heart failure, heart transplantation.

In some embodiments, the disease treatable with the methods disclosed herein is a metabolic disorder including for example type II diabetes, obesity, insulin resistance, sarcopenia, diabetes, acute liver failure, NASH, hepatosteatosis, alcoholic fatty liver, renal failure and chronic kidney disease.

In some embodiments, the disease treatable with the methods disclosed herein is a neurodegenerative diseases including for example Alzheimer's disease, Lewy body dementia, frontotemporal dementia, traumatic brain injury, prion diseases, Huntington's disease, Parkinson's disease, chronic traumatic encephalopathy, amyotrophic lateral sclerosis, mixed dementias, vascular dementia, hydrocephalus, and amyotrophic lateral sclerosis.

Also disclosed in this patent document is the use of a therapeutically effective amount of a compound of Formula I or a pharmaceutically acceptable salt thereof to treat a disease or condition. This patent document further provides a therapeutically effective amount of a compound of Formula I or a pharmaceutically acceptable salt thereof for use in the treatment of a disease or condition. The disease or condition, the means of administration, the dosage form and formulation, and the additional agents are the same as in the methods described herein.

Another aspect of the patent document discloses a method of inhibiting mitofusin 1 and/or mitofusin 2. The method is applicable to attenuating or inhibiting mitofusin-mediated mitochondrial fusion. The method includes contacting a cell containing mitofusin 1 and/or mitofusin 2 with an effective amount of the compound of formula (I) or the pharmaceutically acceptable salt thereof or pharmaceutical composition disclosed herein to inhibit mitofusin 1 and/or mitofusin 2. The compounds are also effective for promoting decreased mitochondrial respiration and functionality, decreased metabolites of TCA cycle and/or promoting mitochondrial outer membrane permeabilization that leads to sublethal caspase activation and DNA damage. In some embodiments, the contacting takes place in vitro. In some embodiments, the contacting takes place in vivo. Similarly, this patent document further provides an effective amount of a compound of Formula I or a pharmaceutically acceptable salt thereof for use in inhibiting mitofusin-mediated mitochondrial fusion. Also disclosed in this patent document is the use of an effective amount of a compound of Formula I or a pharmaceutically acceptable salt thereof to inhibit mitofusin-mediated mitochondrial fusion.

A related aspect provides a method of inducing or promoting apoptosis/cell death in tumor cells. The method includes contacting a cell with an effective amount of the compound of formula (I) or the pharmaceutically acceptable salt thereof or pharmaceutical composition disclosed herein. In some embodiments, the contacting takes place in vitro, or the compound or its salt is administered to a subject in need thereof. The scope of cancer is as descried above. In some embodiments, the cancer is leukemia, melanoma, pancreatic cancer, colon cancer, lung cancer, head and neck cancer, lymphoma, ovarian cancer, prostate cancer, breast cancer, kidney cancer, liver cancer, or bladder cancer. As illustrated in the examples, a compound of Formula I or its salt inhibits MFN1/2 and/or fragmentation of mitochondria and is able to induce a robust apoptosis induction and cell death.

In some embodiments of any methods or uses disclosed herein, there includes the administration of an additional agent including for example SMAC mimetics, BH3 mimetics and other pro-apoptotic anti-cancer drugs. BH3 mimetics is a class of small molecules that antagonizes with the pro-apoptotic BH3 domains in the binding to the hydrophobic pocket of the anti-apoptotic BCL-2 family of proteins such as Bcl-2, Bcl-xL, Mcl-1. BH3 mimetics are essentially selective inhibitors of Bcl-2 or Bcl-xL or Mcl-1 proteins or inhibit more than one anti-apoptotic BCL-2 family of proteins and activate the intrinsic pathway of apoptosis by inducing mitochondrial outer membrane permeabilization (MOMP). BH3 mimetics have been used as an anti-cancer treatment in the clinic in various solid tumors and hematological malignancies as they induce cell death/apoptosis in cancer cells. SMAC mimetics is a class of small molecules that mimics the interaction of second mitochondria-derived activator of caspases (SMAC) with the inhibitor of apoptosis proteins (IAPs). SMAC mimetics are antagonists of cIAP1, cIAP2, XIAP proteins. Inhibition of IAPs by SMAC mimetics induces cell death/apoptosis in cancer cells by activating the intrinsic and/or the extrinsic pathway of apoptosis. SMAC mimetics are evaluated in the clinic as anti-cancer treatment against solid tumors and hematological malignancies.

Nonlimiting examples of SMAC mimetics include Birinapant (TL32711), GDC-0152, Xevinapant (AT406), Tolinapant (ASTX660), AZD5582, BV-6, SM-164, LCL161, and APG-1387. Nonlimiting examples of BH3 mimetics include Venetoclax (ABT-199), Lisaftoclax (APG-2575), S55746, DT2216, Navitoclax (ABT-263), ABT-737, APG-1252, A-1331852, A-115546, S64315 (MIK665), S63845, AMG-176 and AZD5991. When used in combination, the SMAC mimetic or BH3 mimetic can be administered prior to, simultaneously with, or subsequent to the administration of the compound of Formula I or a salt thereof. In some embodiments, the compound of Formula I or a salt thereof in the combination is in an effective amount to decrease cell viability by about 10%, about 20%, about 30%, about 40%, about 50%, about 60%, about 80%, or about 100% in comparison with the SMAC mimetic or BH3 mimetic administered by itself. In some embodiments, the compound of Formula I or a salt thereof in the combination is in an effective amount to increase caspase 3/7 activation by about 10%, about 20%, about 30%, about 40%, about 50%, about 60%, about 80%, or about 100% in comparison with the SMAC mimetic or BH3 mimetic administered by itself.

Another aspect of the patent document provides a method of sensitizing cells to caspase activation or to apoptosis/cell death. The method includes contacting a cell with an effective amount of the compound of formula (I) or the pharmaceutically acceptable salt thereof or pharmaceutical composition disclosed herein. In some embodiments, the method improves caspase activation. In some embodiments, the method sensitizes the cells to improve the response to a SMAC mimetic or BH3 mimetic or other pro-apoptotic drug or agent. The compound of Formula I or a salt thereof can be administered prior to, simultaneously with, or subsequent to the administration of the SMAC mimetic or BH3 mimetic or other pro-apoptotic drug. In some embodiments, the method includes contacting the compound or agent with a cell in vitro. In some embodiments, the method includes contacting the compound or agent with a cell in vivo or administering the compound or agent to a subject.

In some embodiments of any method disclosed herein, the compound of Formula I or a salt thereof in the combination is in an effective amount to decrease cell viability by about 10%, about 20%, about 30%, about 40%, about 50%, about 60%, about 80%, or about 100% in comparison with a vehicle alone. In some embodiments, the compound of Formula I or a salt thereof in the combination is in an effective amount to increase caspase 3/7 activation by about 10%, about 20%, about 30%, about 40%, about 50%, about 60%, about 80%, or about 100% in comparison with a vehicle alone. In some embodiments of any method disclosed herein, the compound of Formula I or a salt thereof in the combination is in an effective amount to sensitize cells to apoptosis/cell death to other pro-apoptotic molecules (e.g. SMAC mimetic or BH3 mimetic, etc.) by about 10%, about 20%, about 30%, about 40%, about 50%, about 60%, about 80%, or about 100% in comparison with a vehicle or the pro-apoptotic molecule.

### Administration Regimen

The compound of Formula I, or a pharmaceutically acceptable salt thereof or a pharmaceutically composition thereof for the methods or kit described herein described herein may be administered to the subject by any suitable means. Non-limiting examples of methods of administration include, among others, (a) administration though oral pathways, which administration includes administration in capsule, tablet, granule, spray, syrup, or other such forms; (b) administration through non-oral pathways such as rectal, vaginal, intraurethral, intraocular, intranasal, or intrarticular, which administration includes administration as an aqueous suspension, an oily preparation or the like or as a drip, spray, suppository, salve, ointment or the like; (c) administration via injection, subcutaneously, intraperitoneally, intravenously, intramuscularly, intradermally, intraorbitally, intracapsularly, intraspinally, intrasternally, or the like, including infusion pump delivery; as well as (d) administration topically; as deemed appropriate by those of skill in the art for bringing the active compound into contact with living tissue.

Advantageously, the compound of Formula I, or a pharmaceutically acceptable salt thereof or a pharmaceutically composition thereof for administrations described above are prepared into dosage forms in a unit dose suited to fit a dose of the active ingredients. Such dosage forms in a unit dose include, for example, tablets, pills, capsules, injections (ampoules), suppositories, etc.

In exemplary embodiments of the pharmaceutical composition of the compound of Formula I, or a pharmaceutically acceptable salt thereof for oral administration, the composition can be a tablet, coated tablet, capsule, caplet, cachet, lozenges, gel capsule, hard gelatin capsule, soft gelatin capsule, troche, dragee, dispersion, powder, granule, pill, liquid, an aqueous or non-aqueous liquid suspension, an oil-in-liquid or oil-in-water emulsion, including sustained release formulations that are known in the art. For pediatric and geriatric applications, suspensions, syrups and chewable tablets are especially suitable.

The therapeutically effective amount (dosage) of the compound of Formula I, or a pharmaceutically acceptable salt thereof required will depend on the route of administration, the species (human or animal), and the physical characteristics of the particular subject or patient being treated. The dose can be tailored to achieve a desired effect, but will depend on such factors as weight, diet, concurrent medication and other factors which those skilled in the medical arts will recognize. More specifically, a therapeutically effective amount means an amount of compound effective to prevent, alleviate or ameliorate symptoms of disease or prolong the survival of the patient or animal being treated. Determination of a therapeutically effective amount is well within the capability of those skilled in the art, especially in light of the detailed disclosure provided herein.

In non-human animal studies, applications of potential products are commenced at higher dosage levels, with dosage being decreased until the desired effect is no longer achieved or adverse side effects disappear. The dosage may range broadly, depending upon the desired effects and the therapeutic indication. Typically, dosages may be about 10 μg/kg to about 100 mg/kg body weight, preferably about 100 μg/kg to about 10 mg/kg body weight. Alternatively, dosages may be based and calculated upon the surface area of the animal, as understood by those of skill in the art.

The exact formulation, route of administration and dosage for the pharmaceutical compositions can be chosen by the individual physician in view of the patient's condition. (see e.g., Fingl et al. 1975, in “The Pharmacological Basis of Therapeutics”, which is hereby incorporated herein by reference in its entirety, with particular reference to Ch. 1, p. 1). In some embodiments, the dose range of the compound of Formula I or a pharmaceutically acceptable salt thereof administered to the subject or patient can be from about 0.5 to about 1000 mg/kg of their body weight. The dosage may be a single one or a series of two or more given in the course of one or more days, as is needed by the patient. In instances where human dosages for compounds have been established for at least some conditions, those same dosages, or dosages that are about 0.1% to about 500%, more preferably about 25% to about 250% of the established human dosage may be used.

It should be noted that the attending physician would know how to and when to terminate, interrupt, or adjust administration due to side-effects, toxicity or organ dysfunctions. Conversely, the attending physician would also know to adjust treatment to higher levels if the clinical response was not adequate (precluding toxicity). The magnitude of an administrated dose in the management of the disorder of interest will vary with the severity of the condition to be treated and to the route of administration. The severity of the condition may, for example, be evaluated, in part, by standard prognostic evaluation methods. Further, the dose and perhaps dose frequency will also vary according to the age, body weight, and response of the individual patient. A program comparable to that discussed above may also be used in veterinary medicine.

Although the exact dosage will be determined on a drug-by-drug basis, in most cases, some generalizations regarding the dosage can be made. The daily dosage regimen for an adult human patient may be, for example, a peroral dose of about 0.01 mg to 2000 mg of the active ingredient, preferably from about 0.01 mg to about 500 mg. In other embodiments, an intravenous, subcutaneous, or intramuscular dose of the active ingredient of about 0.01 mg to about 100 mg, preferably about 0.01 mg to about 60 mg is used. In cases of administration of a pharmaceutically acceptable salt, dosages may be calculated as the freebase. In some embodiments, the composition is administered 1 to 4 times per day. Alternatively, a compound of Formula I or a pharmaceutically acceptable salt thereof may be administered by continuous intravenous infusion, preferably at a dose of up to about 1000 mg per day. As will be understood by those of skill in the art, in certain situations it may be necessary to administer a compound of Formula I or a pharmaceutically acceptable salt thereof disclosed herein in amounts that exceed, or even far exceed, the above-stated, preferred dosage range in order to effectively and aggressively treat particularly intractable diseases or conditions. In some embodiments, a compound of Formula I or a pharmaceutically acceptable salt thereof will be administered for a period of continuous therapy, for example for a week or more, or for months or years.

In some embodiments, a compound of Formula I or a pharmaceutically acceptable salt thereof is formulated into a dosage form for release for a period of 1 to 12, typically 3 to 12 hours, more typically 6-12 hours after administration. In some embodiments, the oral pharmaceutical compositions described herein may be administered in single or divided doses, from one to four times a day. The oral dosage forms may be conveniently presented in unit dosage forms and prepared by any methods well known to those skilled in the art of pharmacy.

A compound of Formula I or a pharmaceutically acceptable salt thereof can be evaluated for efficacy and toxicity using known methods. For example, the toxicology of the compound may be established by determining in vitro toxicity towards a cell line, such as a mammalian, and preferably human, cell line. The results of such studies are often predictive of toxicity in animals, such as mammals, or more specifically, humans. Alternatively, the toxicity may be determined in an animal model (such as mice, rats, rabbits, or monkeys) using known methods. The efficacy of a particular compound may be established using several recognized methods, such as in vitro methods, animal models, or human clinical trials. Recognized in vitro models exist for nearly every class of condition. Similarly, acceptable animal models may be used to establish the efficacy of chemicals to treat such conditions. When selecting a model to determine efficacy, the skilled artisan can be guided by the state of the art to choose an appropriate model, dose, and route of administration, and dosing regime. Of course, human clinical trials can also be used to determine the efficacy of a compound of Formula I or a pharmaceutically acceptable salt thereof in humans.

A compound of Formula I or a pharmaceutically acceptable salt thereof may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration. The pack or dispenser may also be accompanied with a notice associated with the container in a form prescribed by a governmental agency regulating the manufacture, use, or sale of pharmaceuticals, which notice is reflective of approval by the agency of the form of the drug for human or veterinary administration. Such notice, for example, may be the labeling approved by the U.S. Food and Drug Administration for prescription drugs, or the approved product insert. Compositions comprising a compound of Formula I or a pharmaceutically acceptable salt thereof formulated in a compatible pharmaceutical carrier may also be prepared, placed in an appropriate container, and labeled for treatment of an indicated condition.

Assays that can be used to monitor compound-induced inhibition of mitochondrial fusion in a mitofusin dependent manner include, for example, fluorescent microscopy to monitor mitochondrial shape and measurement of mitochondrial aspect ratio, EM microscopy to monitor mitochondrial shape, PEG mitochondrial fusion assay, and In vitro mitochondrial fusion luminescent and fluorescent-based assays.

The following non-limiting examples serve to further illustrate the embodiments of the present disclosure.

### EXAMPLES

Reagents. MASM7 was obtained from Enamine (cat. #EN300-396282). Screened MASMs were purchased from Enamine, ChemBridge and ChemDiv. MFI8 was obtained from ChemBridge (cat. #7681311) and also synthesized in house in a more stable form as a HCl salt. MFI8 was freshly dissolved in DMSO at 10 mM prior each experiment. MFI22-26 were also synthesized in house as HCl salts. The rest of the screened MFIs were purchased from Enamine, ChemBridge, ChemDiv, Vitas M and UORSY. All compounds were >95-98% pure, dissolved in 100% DMSO to prepare a 10 mM stock solution and diluted in aqueous buffers or cell culture medium for assays. BV6 (B4653) and Q-VD-Oph (A1901) were purchased from APExBio. Staurosporine (S1421) was purchased from Selleck. Myxothiazol (T5580) was purchased from Sigma. Rotenone was purchased from Tocris (36165).

Cell lines. Cell lines were purchased from ATCC. MEFs (WT, MIFN1 KO, MFN2 KO and MFN1/MFN2 DKO) were also provided by David Chan's laboratory. DRP1^{flox/flox} MEFs were provided from Richard Kitsis' laboratory. U2OS cells were provided from Stephen Tait's laboratory. All cells maintained in DMEM (Life Technologies) supplemented with 10% FBS, 100 U ml⁻¹ penicillin/streptomycin and 2 mM L-glutamine.

Mice. All animal experiments were approved by and performed in compliance with the guidelines and regulations approved by the Institutional Animal Care and Use Committee of the Albert Einstein College of Medicine.

Structural model of MFN2. The structural model of MFN2 was calculated based on the I-TASSER (Iterative Threading ASSEmbly Refinement) hierarchical approach to protein structure as we previously described and truncated crystal structure of MFN2 (PDB ID: 6JFL). Energy minimization and analysis of the top-ranked structure was performed with MAESTRO tools (Maestro, Schrödinger Release 2016-2020, Schrödinger, LLC). PyMOL (The PyMOL Molecular Graphics System. Schrödinger Release 2016-2020, Schrödinger, LLC) was used for preparing the figures.

Molecular dynamics simulations. Molecular dynamics (MD) simulations of the full-length MFN2 model structure was performed using DESMOND (Schrodinger LLC, 2021). Three independent 250 ns MD simulations were performed in a truncated TIP3P water box using OPLS3 force field and the NPT ensemble at 300K and constant pressure of 1.0325 bar. The Nose-Hoover Chain thermostat and Martyna-Tobias-Klein barostat were used to maintain the temperature and pressure, respectively. The system was neutralized by Na⁺ and Cl⁻ ions at a final concentration of 0.15 M. Before production runs, the system was minimized and pre-equilibrated using the standard equilibration protocol implemented in DESMOND. Analysis of the trajectories was performed using MAESTRO simulation event analysis tools (Schrodinger LLC, 2021). Interatomic distance plots obtained from MAESTRO were plotted using GraphPad Prism 9. PyMOL (Schrodinger LLC, 2021) was used to show structures of the MD trajectory snapshots.

In silico small molecule library preparation. eMolecules (www.emolecules.com) library of purchasable compounds was converted to 3D structures using LIGPREP (LigPrep, Schrödinger Release 2016, Schrödinger, LLC) and EPIK (Epik, Schrödinger Release 2016, Schrödinger, LLC) generating an in silico library of approximately 13.8 million compounds containing compounds with different ionization state at pH 7.0±2.0, stereochemistry and tautomeric form, excluding potential Pan Assay Interference Compounds (PAINS) using PAINS definitions included in Canvas. Conformation analysis of ligands was calculated using the OPLS3 force field.

3D Pharmacophore model generation and screen. Phase (Phase, Schrödinger Release 2016, Schrödinger, LLC) module was used to generate a pharmacophore hypothesis and a 3D pharmacophore screen. The coordinates of the HR1 helix residues Val372, Met376 and His380 from the structural model of MFN2 were used to assign pharmacophore points in 3D coordinates. Pharmacophore hypothesis included 5 features as defined in Phase for 3 hydrophobic groups to mimic the sidechain residues of Val273 and Met376 and an aromatic ring with a hydrogen-bond donor to mimic the sidechain of His380. The pharmacophore screen used the in silico library of compounds prepared from the eMolecules library in pre-existing conformations with the requirement to satisfy at least 4 out of the 5 pharmacophore features of the hypothesis. The top 1000 compounds ranked based on the Phase Score were selected for further visual analysis and clustered for diversity using dendritic fingerprints in Canvas. Physicochemical and AMDET properties including Lipinski rules, permeability, log P, metabolic liabilities and hERG inhibition were evaluated using QikProp (QikProp, Schrödinger Release 2016, Schrödinger, LLC). The highest 8 ranked compounds and the 10 most diverse compounds yielded selected molecules for experimental validation. MASM7 and MFI8 were checked for potential Pan Assay Interference Compounds (PAINS) and has not been reported as a hit in previous screens in Pubchem database.

Live cell imaging. MEFs were seeded on chamber slides (MatTek Corporation: 35 mm dishes, No. 1.5, 14 mm glass diameter) at ~70% confluence. Cells were treated with MASM7 or MFI8. Then, cells were stained with MitoTracker Green (200 nM, Invitrogen) for 20 min at 37° C. After treatments, media was replaced with FluoroBrite™ DMEM (Invitrogen) supplemented with 10% FBS, 100 U ml⁻¹ penicillin/streptomycin, 2 mM L-glutamine and MASM7 or MFI8 prior image acquisition. Images were taken with Leica SP5 inverted confocal microscope. Data were analyzed with Image J software. >200 mitochondria were measured per condition in mitochondrial aspect ratio.

Measurement of mitochondrial aspect ratio (Mito AR). Images that depict stained mitochondria with mitotracker green were analyzed using FIJI We measured for each mitochondrion its length and width. Then, we divided the length by the width for each mitochondrion to get its mitochondrial aspect ratio. >200 mitochondria were measured per treatment.

Immunofluorescent analysis and microscopy. Cells were treated with MASM7, MFI8 and or Q-VD-OPh at ~70% confluence for 6 h. After treatments, cells were washed with PBS and fixed with 4% PFA for 15 min. Then cells were permeabilized with 0.1% Triton X-100 in PBS for 10 min. Cells were blocked with 5% BSA in PBS-T and incubated overnight with primary antibodies as indicated; γH2AX (Sigma; 05-636, 1:100 dilution). After incubation with primary antibodies, cells were washed with PBS and incubated with the appropriate mouse secondary antibody (ThermoFisher Scientific; A-11001) in the blocking solution. After PBS washes, coverslips were dipped in water and mounted on glass slides using Vectashield containing DAPI (Vector laboratories). Images were taken with Leica SP5 inverted confocal microscope. Data were analyzed with ImageJ.

Mutagenesis. Mutagenesis on the HR2 domain for the NMR analysis or the MFN2 gene that was used for the packaging of the adenovirus was performed by using QuikChange Lightning Site-Directed mutagenesis Kit (Qiagen; Cat. 210518). The following primers were used:

S685A (adeno):
5′-CTGGCTCCAACTGCGCCCACCAAGTCCAGC-3′
L692A (adeno):
5′-CCAAGTCCAGCAGGAAGCGTCTGGGACCTTTGC-3′
S685A (NMR):
5′-TGGGTAGCAACTGCGCCCACCAGGTGCAGC-3′
L692A (NMR):
5′-AGGTGCAGCAAGAGGCGAGCGGCACCTTCG-3′
D725A/L727A:
5′-AAGAAAATTGAAGTTCTGGCCAGCGCGCAAAGCAAGGCGAAACT
G-3′
L727A:
5′-AATTGAAGTTCTGGACAGCGCGCAAAGCAAGGCGAAACT-3′

Recombinant HR2 protein production. Human MFN2 residues 678-757 corresponding to the HR2 domain were cloned into a pET-28 vector fused to a His-tag and transformed into BL21(DE3) CodonPlus (DE3)-RIPL E. coli cells. Cells were grown at 37° C. in 1 L of LB media to an OD₆₀₀ of 0.8, cells were then harvested and resuspended in 1 L of Luria Broth media or M9 media supplemented with 1.5 gr/L of ¹⁵N ammonium chloride grown for 45 min at 37° C. and induced at 18° C. for 16 hours with 1 mM isopropyl β-d-1-thiogalactopyranoside. MFN2-HR2 or ¹⁵N-MFN2-HR2 domain was purified from bacterial pellets by high-pressure homogenization in lysis buffer (20 mM Tris·HCl pH 7, 250 mM KCl, 25 mM imidazole, and Roche complete EDTA free protease inhibitor cocktail) and ultracentrifuged at 45,000 g for 45 min. The supernatant was applied to pre-equilibrated 1 mL HisPur Ni-NTA Resin washed in lysis buffer and eluted using elution buffer (20 mM Tris·HCl pH 6, 250 mM KCl, 400 mM imidazole). MFN2-HR2 or ¹⁵N-MFN2-HR2 was further purified by size exclusion chromatography (Superdex 75 Increase 10/300 GL column) in gel filtration buffer (20 mM potassium phosphate pH 6, 150 mM KCl). Fractions containing the MFN2-HR2 domain were confirmed by SDS-PAGE, pooled and concentrated to 50 M in NMR buffer (20 mM potassium phosphate pH 6, 150 mM KCl, 10% D20) using a 10 KDa cut-off Centricon spin concentrator (Millipore) for prompt use in biochemical and NMR studies.

NMR experiments. The uniformly ¹⁵N-labeled protein samples were prepared by growing the bacteria in a minimal medium, as described above. Correlation ¹H-¹⁵N-HSQC spectra of 50 M MFN2-HR2 in the presence and absence of MASM7 or MFI8 or 367-384Gly or 398-418Gly were recorded on a BRUKER AVANCE IIIHD 600 MHz system equipped with a 5 mm H/F-TCI CryoProbe at 25° C. All experiments were performed using an independent sample for each experimental measurement as a 400 μL sample in a 5-mm Shigemi; all samples were DMSO matched with 2% d₆-DMSO. Spectra were processed using qMDD (mddnmr v2.0) and NMRPIPE and analyzed using Analysis (CCPNMR).

Microscale Thermopheresis. Freshly purified His-tagged MFN2-HR2 domain was used for Microscale Thermophoresis (MST) binding studies. For protein labelling, a fresh stock of 5 M His-tag-RED-tris-NTA 2^{nd} generation dye (Nanotemper) in 25 mM Hepes pH 7.5, 100 mM NaCl, 0.005% Tween-20 (assay buffer) was used to label 500 nM of MFN2-HR2 in the same buffer. The labeling reaction was incubated for 30 min at RT and centrifuged at 15.000×g at 4° C. for 10 min. Labelled protein from the supernatant was kept on ice and used immediately. Compounds were serially diluted in 100% DMSO. Immediately before mixing with labeled protein, the dilution series was transferred in assay buffer to reach 2% DMSO. 10 μl of each compound in the final series was mixed with 10 l of labeled protein and samples were incubated for 5 min at 30° C. before MST measurement using a MonolithNT.115 instrument (Nanotemper). Peptides were diluted in assay buffer plus 2% DMSO and treated as above. Final MST conditions: 25 mM Hepes pH 7.5, 100 mM NaCl, 0.005% Tween-20, 1% DMSO, 125 nM labeled HR2, 25 nM NTA dye, temperature 30° C., LED power 40%, MST power Medium, before MST 3 sec, MST-on, 10 sec, after MST: 1 sec. Samples were measured in standard capillaries (Nanotemper) and showed no aggregation according to post-run analysis using the Monolith data collection software (Nanotemper). Kd values were obtained from non-linear regression fits of normalized data to a three or four parameter logistic curve, using GraphPad Prism 9.

In vitro MFN2 oligomerization. MEFs were seeded in 8×15 cm² dishes and grown at ~90% confluence. Then, mitochondria were isolated according to previously published protocol. Briefly, cells were harvested, pelleted and washed with cold PBS. Then, cells were resuspended in cold mitochondrial isolation buffer (0.2 M sucrose, 10 mM Tris-MOPS pH 7.4, 1 mM EGTA, 5 mM Mg(OAc)₂, 50 mM KOAc, 1×HALT protease inhibitors, 0.5 mM PMSF) and homogenized in dounce homogenizer with 20 strokes. Isolated mitochondria were incubated with 2 mM GTP, 10 μM MASM7 and 40 M MFI8 at 37° C. for 30 min. Equal volume of 2× lysis buffer was added to each reaction to have final concentration of (50 mM Bis-Tris, 50 mM NaCl, 10% Glycerol, and 1% wt/vol Digitonin), then samples were incubated on ice for 15 min. Lysates were centrifuged at 16,000 g at 4° C. for 30 min. Subsequently, supernatant was mixed with NativePAGE 5% G-250 Sample Additive to a final concentration of 0.25%. Proteins were resolved with a Novex NativePAGE 4-16% Bis-Tris Protein Gels (Invitrogen) at 4° C. according to previously published protocol.

Blue native PAGE. The procedure for the Blue native PAGE (BN-PAGE) electrophoresis was adapted from previously published protocol. Lysates from the in vitro MFN2 oligomerization experiment were resolved with a Novex Native PAGE 4-16% Bis-Tris Protein Gels (Invitrogen) at 4° C. The gels were firstly run in the dark cathode buffer at 40 V for 30 min and then at 100 V for 30 min. Then, the dark cathode buffer was replaced with light cathode buffer and the gels were run at 100 V for 30 min and at 200 V for 1 hr and 50 min. Next, the gels were transferred to polyvinylidene fluororide (PVDF) membranes at 30 V for 16 h using a transfer buffer (Tris 25 mM, 192 mM glycine, 20% methanol). Once the transfer was completed, the membranes were incubated 8% acetic acid for 15 min and subsequently washed with water for 5 min. Then, membranes were dried at 37° C. for 20 min, rehydrated in 100% methanol, and washed with water. Finally, membranes were blocked for 1 hr in PBS-T containing 5% milk and 1% BSA and incubated overnight with an anti-MFN2 antibody (Cell Signaling; #9482S) at a 1:1000 dilution.

Densitometric analysis. Densitometric data from western blot scanned films were obtained using Image Studio software (LI-COR). Data were corrected to loading control (total ACTIN) and normalized to the treated bands that correspond to 25° C. (100%) and blot backgrounds (0%). Tm values were obtained from non-linear regression fits of normalized data to a four-parameter logistic curve (4PL), using GraphPad Prism 8.

Western blot. Western blots were performed from whole cell lysates (WCL) prepared in lysis buffer containing 50 mM Tris-HCl pH 7.5, 1% Triton X-100, 150 mM NaCl, 5 mM MgCl₂, 1 mM EGTA and 10% glycerol in the presence of protease inhibitor cocktail (Roche). WCL were separated on a 4-12% NuPAGE MES gel (Invitrogen), transferred into a PVDF membrane, block for 1 hr and immunoblot with the corresponding anti-bodies.

Mitochondrial depolarization assay (mtΔΨ). MEFs (10⁴ cells/well) were seeded in a 96-well black plate and treated with MASM7 or MFI8 for 6 hrs. Following treatments, cells were stained with 250 nM TMRE (Sigma; Cat. 87917) for 20 min at 37° C. Subsequently, cells were washed with thrice with PBS. Fluorescence intensity was detected by a M100 microplate reader (TECAN, Ex: 540 nm/Em: 579 nm).

Mitochondrial respiration in cellulo. Mitochondrial oxygen consumption rates (OCR) were assessed using a XF24 Analyzer (Seahorse Biosciences, Billerica MA, USA). In brief, 3×10⁴ MEFs were cultured in a XF24 cell culture microplate containing DMEM supplemented with 10% fetal bovine serum. Cells were treated with MASM7 (1 μM) or MFI8 (20 μM) 6 hrs prior to OCR analysis. Mitochondrial respiration was assessed by the sequential addition of oligomycin (1 μM), carbonyl cyanide-4-(trifluoromethoxy)phenylhydrazone (FCCP, 2 μM) and rotenone (1 μM)-antimycin (1 μM) as previously reported. OCR was normalized to cell number or total protein content per well for each condition tested.

Mitochondrial respiration in isolated mitochondria. Mitochondria were isolated based on previously published protocol. Isolated mitochondria were treated with MFI8 (20 μM) for 30 min prior to OCR analysis. OCR was measured using the Mitocell (MT200), a Clarktype electrode from Strathkelvin instruments. Isolated mitochondria (50 g) from murine cardiomyocytes were loaded into the 50 μL magnetically stirred respiration chamber containing EBm buffer (1 M sucrose, 0.01 M Tris/HCl, 1 M MgCl₂, 0.1 M EGTA/Tris, 2 mM KH₂PO₄, pH 7.4). Glutamate and malate were added to the vessel at final concentrations of 0.25 M and 0.125 M, respectively. Respiration was measured after the addition of substrates (glutamate and malate) and termed “state 2 respiration”. ADP was then added at a final concentration of 800 μM to measure “state 3 respiration.” Addition of oligomycin at final concentration 800 ng/ml was used to quench the reaction and measure proton leakage. Finally, FCCP was added at final concentration 200 nm to measure maximum (uncoupled) respiration.

Caspase-3/7 activation assay. Cells (5×10³ cells/well) were seeded in a 96-well white plate and treated with MASM7, MFI8. Caspase 3/7 activation was measured after 6 hr by addition of the Caspase-Glo 3/7 reagent according to the manufacturer's protocol (Promega). Luminescence was detected by a F200 PRO microplate reader (TECAN). Caspase assays were performed in at least triplicate and the data normalized to vehicle-treated control wells. Dilutions of MASM7 or MFI8 were performed using a TECAN D300e Digital Dispenser from 10 mM stocks.

Cytochrome C release. MEFs were seeded in a 10 cm² dish and grown at ~70% confluence. Following 6 hrs treatment with MFI8, cells were harvested and lysed in digitonin buffer [20 mM Hepes, pH 7.2, 10 mM KCl, 5 mM MgCl₂, 1 m MEDTA, 1 mM EGTA, 250 mM sucrose, 0.025% Digitonin (from 5% w/v stock) and complete protease inhibitors (Roche Applied Science)] on ice for 10 min. Then, supernatants were isolated by centrifugation at 15,000×g for 10 min. Mitochondrial pellets solubilized in 1% Triton X-100/PBS for 1 hr at 4° C. Solubilized Pellets were subjected to a 14,000×rpm spin for 10 min. Samples were prepared for western blot analysis and separated by 4-12% NuPage (Life Technologies).

Cell viability assay. Cells (5×10³ cells/well) were seeded in a 96-well white plate and treated with serial dilutions of MASM7 or MFI8. Cell viability was assayed after 72 hrs by addition of CellTiter-Glo reagent according to the manufacturer's protocol (Promega). Luminescence was measured using a F200 PRO microplate reader (TECAN). Viability assays were performed in at least triplicate and the data normalized to vehicle-treated control wells. Dilutions of MASM7 or MFI8 were performed using a TECAN D300e Digital Dispenser from 10 mM stocks.

Cell death assay. Cells (2×10⁵ cells/well) were seeded in a 6-well plate and treated with the indicated drugs for 6 hours. Cells were dissociated using Accutase (Thermo Fisher; Cat. #00-4555-56) in order to avoid accidental exposure of phosphatidylserine on the outer plasma membrane. Cell death was evaluated with Dead Cell Apoptosis Kit with Annexin V Alexa Fluor™ 488 & Propidium Iodide (Thermo Fisher; Cat. #V13241) according to manufacturer's protocol. Data was acquired by BD LSRII flow cytometer system using BD FACSDiva software. Data was analyzed by FlowJo (BD).

Metabolomics. MEFs were treated with MASM7 and MFI8. Cells were harvested using a cell scraper and centrifuged at 1200 rpm in 4° C. for 3 min. Each cell pellet sample was suspended into 250 to 700 μL of 80% aqueous methanol in an Eppendorf tube. The samples were vortex mixed for 15 s and sonicated in an ice-water bath for 5 min, followed by centrifugal clarification at 15,000 rpm and 5° C. in an Eppendorf 5424R centrifuge. The clear supernatants were collected. A standard stock solution of TCA cycle carboxylic acids, NAD and NADH was prepared in 80% methanol as S1. This standard solution S1 was serially diluted 1 to 4 (v/v) with the same solvent to make standard solutions S2 to S10. 20 μL of each standard solution and an aliquot of the clear supernatant from each cell was mixed with 20 μL of an internal standard solution containing 9 ¹³C- or deuterium labeled analogues of the TCA cycle carboxylic acids (except isocitric acid), 20 μL of 200 mM 3-NPH solution and 20 μL of 150 mM of EDC solution. The mixtures were allowed to react at 30° C. for 30 min. After reaction, 120 μL of water was added to each solution. 10 μL of the resultant solutions was injected into a C18 UPLC column to quantitate the TCA cycle carboxylic acids by UPLC-MRM/MS with (−) ion detection, according to the procedure we described in a publication.

Minority MOMP. U2OS cells (100,000) were seeded onto glass coverslips and transfected with 250 ng each of CytoGFP and Mito-mCherry as previously described 39. 16 h after transfection, cells were treated with compounds for 3 h and then fixed in 4% paraformaldehyde. Cells were imaged on a Zeiss LSM880 with Airyscan using a 63×1.4 NA objective. Subsequent to acquisition, images were processed using the Airyscan processing function in ZEN software. Minority MOMP was quantified by manually scoring the co-localisation of CytoGFP and mito-mCherry.

Analysis of mitochondrial biogenesis-related genes by q-PCR. Total RNA was extracted by using RNeasy Mini Kit (Qiagen) followed by cDNA synthesis using the SuperScript IV VILO Master Mix (Thermo Scientific), both performed according to the manufacturer's protocol. q-PCR was performed using the following primers:

Rpl139 Forward:
CAAAATCGCCCTATTCCTCA
Rpl139 Reverse:
AGACCCAGCTTCGTTCTCCT
Mfn1 Forward:
GACCGAAGGGTCAGATGAAA
Mfn1 Reverse:
TCCAGCTCTGTGGTGACATC
Mfn2 Forward:
CCTGGATGCTGATGTGTTTG
Mfn2 Reverse:
CCAGCTCATCCACCAGAAAG
Ppargc1a Forward:
TGATGTGAATGACTTGGATACAGACA
Ppargc1a Reverse:
GCTCATTGTTGTACTGGTTGGATATG
Ppargc1b Forward:
GGGAAAGGGACCAGACATAATC
Ppargc1b Reverse:
GCGGAAGCAGATGGTAAGATAA

For the q-PCR reaction, Power SYBR Green master mix (Thermo Scientific) was used according to the manufacturer's instructions. Briefly, each reaction consisted of 10 ng cDNA, 5 μL Power SYBR Green master mix, 200 nM primers (forward and reverse), and RNase-free water up to 10 μL. q-PCR was performed on the ViiA 7 Real-Time PCR System (Thermo Scientific) with the following cycle parameter: 95° C. for 10 min, 40 cycles of 95° C. for 15 s, and 60° C. for 1 min. q-PCR products were analyzed by melting curves for unspecific products or primer dimer formation. Rpi39 was used as housekeeping gene and 2^{-ΔΔCT} method was applied to determine the relative mRNAs expression.

Determination of mtDNA to genomic DNA ratio by q-PCR. DNA was extracted using DNeasy Blood and Tissue kit (Qiagen) according to the manufacturer's protocol. q-PCR was used to determine the ratio of mitochondrial DNA (mtDNA) and genomic DNA (gn DNA). The following primers were used for q-PCR reaction:

mt DNA (Nd2) Forward:
CCTATCACCCTTGCCATCAT
mt DNA (Nd2) Reverse:
GAGGCTGTTGCTTGTGTGAC
gn DNA (Pecam1) Forward:
ATGGAAAGCCTGCCATCATG
gn DNA (Pecam1) Reverse:
TCCTTGTTGTTCAGCATCAC

Each reaction consisted of 5 ng of DNA, 5 μL Power SYBR Green master mix (Thermo Scientific), 200 nM primers (forward and reverse), and RNase-free water up to 10 μL. q-PCR was performed on the ViiA 7 Real-Time PCR System (Thermo Scientific) at 95° C. for 10 minutes, 40 cycles of 95° C. for 15 s, and 60° C. for 1 minute, followed by melting curve analysis. 2^{-ΔΔCT} method was applied to determine the mtDNA/nDNA ratio, being ΔCT=CT(mtDNA gene)−CT(nDNA gene).

RNA-seq. Sequencing results were demultiplexed and converted to FASTQ format using Illumina bcl2fastq software. The sequencing reads were adapter and quality trimmed with Trimmomatic and then aligned to the mouse genome (build mm10/GRCm38) using the splice-aware STAR aligner. The featureCounts program was utilized to generate counts for each gene based on how many aligned reads overlap its exons. These counts were then normalized and used to test for differential expression using negative binomial generalized linear models implemented by the DESeq2 R package.

Chemical syntheses. All chemical reagents and solvents were obtained from commercial sources and used without further purification. FastWoRX™ was purchased from Faster Chemistry LLC. Microwave reactions were performed using an Anton Paar Monowave 300 reactor. Chromatography was performed on a Teledyne ISCO CombiFlash R_{f} 200i using disposable silica cartridges. Analytical thin layer chromatography (TLC) was performed on Merck silica gel plates and compounds were visualized using UV. NMR spectra were recorded on a Bruker 600 spectrometers. ¹H chemical and ¹³C chemical shifts (6) are reported relative to tetramethyl silane (TMS, 0.00 ppm) as internal standard or relative to residual solvent signals. Mass spectra were recorded by the Proteomics Facility at the Albert Einstein College of Medicine.

### Preparation of Compound 40-2

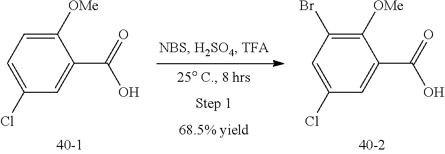 [SMILES: COc1ccc(Cl)cc1C(=O)O ; COc1c(Br)cc(Cl)cc1C(=O)O]

To a solution of compound 40-1 (20.0 g, 107 mmol, 1.00 eq) in H₂SO₄ (40.0 mL) and TFA (80.0 mL) was added NBS (21.0 g, 118 mmol, 1.10 eq). The mixture was stirred at 25° C. for 8 hrs. TLC (DCM:MeOH=5:1, compound 40-1 R_{f}=0.50, compound 40-2 R_{f}=0.30) indicated compound 40-1 was consumed completely, and a major new spot with larger polarity were detected. The reaction mixture was quenched by H₂O 360 mL at 0° C., and extracted with EtOAc 480 mL (240 mL*2). The combined organic layers were washed with brine 300 mL, dried over Na₂SO₄, filtered and concentrated under reduced pressure to give a residue. The crude product was triturated with DCM 120 mL at 25° C. for 30 mins to give compound 40-2 (19.5 g, 73.5 mmol, 68.5% yield) as a white solid.

### Preparation of Compound 40-4

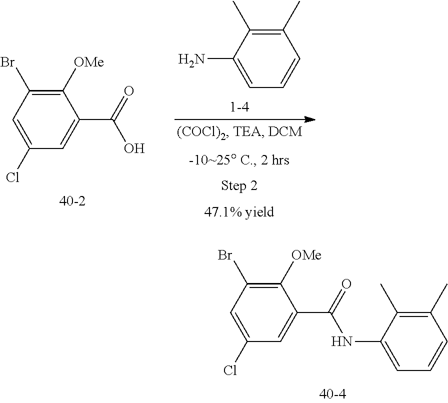

To a solution of compound 40-2 (16.5 g, 62.2 mmol, 1.00 eq) in DCM (180 mL) was added (COCl)₂ (39.4 g, 310 mmol, 5.00 eq) at −10° C. The mixture was stirred at 25° C. for 1 hrs. And then the mixture was concentrated under reduced pressure to give a residue. The residue was added into a solution of compound 1-4 (9.79 g, 80.8 mmol, 1.30 eq) and TEA (12.6 g, 124 mmol, 2.00 eq) in DCM (200 mL). The mixture was stirred at 25° C. for 1 hr. LCMS (EC14613-10-P1A2, product: RT=0.443 min) showed compound 40-2 was consumed and 62.7% of compound 40-4 was detected. The reaction mixture was filtered and concentrated under reduced pressure to give a residue. The residue was purified by prep-HPLC (FA condition) to give compound 40-4 (10.8 g, 28.3 mmol, 47.1% yield) as a yellow solid.

LCMS: EC14613-10-P1A1, product: RT=0.443 min, m/z=369.9 [M+H]⁺

¹H NMR: EC14613-13-P1N1, (400 MHz, CDCl₃)

δ 9.49-9.39 (m, 1H), 8.17-8.10 (m, 1H), 7.89-7.81 (m, 1H), 7.77-7.69 (m, 1H), 7.21-7.15 (m, 1H), 7.09-7.04 (m, 1H), 2.35 (s, 3H), 2.26 (s, 3H)

### Preparation of Compound 40-6

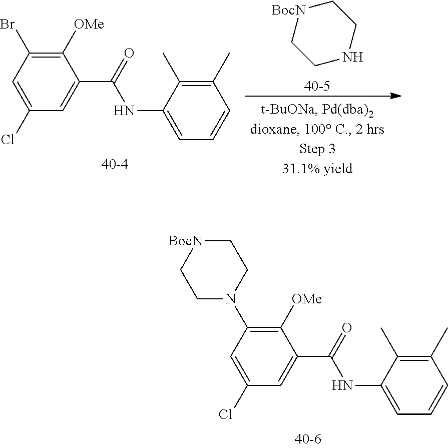

A mixture of compound 40-4 (1.00 g, 2.71 mmol, 1.00 eq), compound 40-5 (581 mg, 3.12 mmol, 1.15 eq), t-BuONa (782 mg, 8.14 mmol, 3.00 eq), Pd(dba)₂ (156 mg, 271 mol, 0.10 eq) in dioxane (20.0 mL) was degassed and purged with N₂ for 3 times, and then the mixture was stirred at 100° C. for 2 hrs under N₂ atmosphere. LCMS (EC14613-14-P1A1, product: RT=2.811 mins) showed compound 40-4 was consumed completely and 20.7% of compound 40-6 was detected. The reaction mixture was quenched by H₂O 30.0 mL at 25° C., and then diluted with EtOAc 30.0 mL and extracted with EtOAc 150 mL (50.0 mL*3). The combined organic layers were washed with brine 50.0 mL, dried over Na₂SO₄, filtered and concentrated under reduced pressure to give a residue. The residue was purified by prep-HPLC (basic condition) to give compound 40-6 (400 mg, 843 mol, 31.1% yield) as a yellow solid.

LCMS: EC14613-14-P1A1, product: RT=2.811 mins, m/z=474.1 [M+H]⁺

### Preparation of Compound MFI41

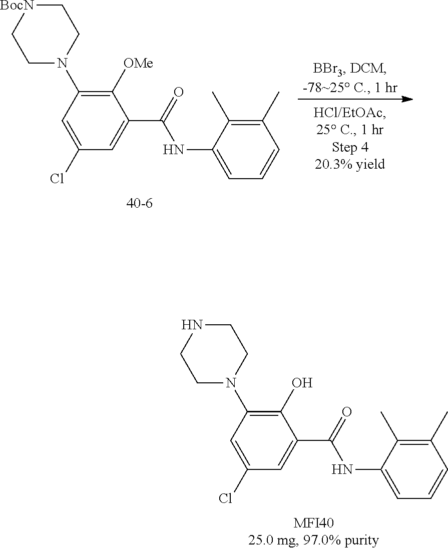

To a solution of compound 40-6 (400 mg, 843 mol, 1.0 eq) in DCM (5.00 mL) was added BBr₃ (317 mg, 1.27 mmol, 1.50 eq) at −78° C. And then the mixture was stirred at 25° C. for 1 hr. TLC (DCM:MeOH=5:1, compound 40-6 R_{f}=0.50, MFI40 R_{f}=0.30) indicated compound 40-6 was consumed completely, and a major new spot with larger polarity were detected. The reaction mixture was quenched by H₂O 30.0 mL at 0° C., and extracted with DCM 75.0 mL (25.0 mL*2). The combined organic layers were washed with brine (80.0 mL), dried over Na₂SO₄ filtered and concentrated under reduced pressure to give a residue. The residue was added HCl/EtOAc (4M, 10.0 mL) and stirred at 25° C. for 1 hr. The mixture was concentrated under reduced pressure to give a residue. The residue was purified by prep-HPLC (neutral condition) to give MFI40 (77.0 mg, 214 mol, 20.3% yield, 96.6% purity) as a yellow solid.

LCMS: EC14613-17-P1A1, product: RT=1.660 mins, m/z=360.1 [M+H]⁺

HPLC: EC14613-17-P1A3, product: RT=1.533 mins, 96.6% purity

¹H NMR: EC14467-17-P1A1, (400 MHz, DMSO-d₄) δ 14.28 (s, 1H), 8.00 (d, J=7.6 Hz, 1H), 7.48 (d, J=2.6 Hz, 1H), 7.00 (t, J=7.8 Hz, 1H), 6.84 (d, J=7.4 Hz, 1H), 6.59-6.53 (m, 1H), 3.25-3.13 (m, 10H), 2.50 (s, 5H), 2.25 (s, 3H), 2.21 (s, 3H)

### Preparation of Compound 41-2

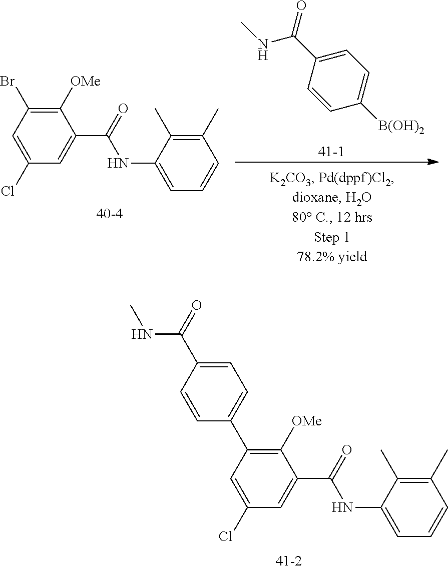

To a solution of compound 40-4 (2.00 g, 5.43 mmol, 1.00 eq) in dioxane (20.0 mL) was added K₂CO₃ (1.50 g, 10.9 mmol, 2.00 eq) in H₂O (5.00 mL), compound 41-1 and Pd(dppf)Cl₂ (794 mg, 1.09 mmol, 0.20 eq) and the mixture was degassed and purged with N₂ for 3 times. Then the mixture was stirred at 80° C. for 12 hrs. LCMS (EC14467-13-P1A1, product: RT=0.467 min) showed compound 40-4 was consumed completely and 71.7% of compound 41-2 was detected. The reaction mixture was quenched by H₂O 30.0 mL at 0° C., and extracted with EtOAc 75.0 mL (25.0 mL*3). The combined organic layers were washed with brine 80.0 mL, dried over Na₂SO₄, filtered and concentrated under reduced pressure to give compound 41-2 (2.50 g, 4.23 mmol, 71.6% purity, 78.2% yield) as a black brown solid.

LCMS: EC14467-13-P1A1, product: RT=0.467 min, m/z=423.1 [M+H]⁺

### Preparation of Compound MFI41

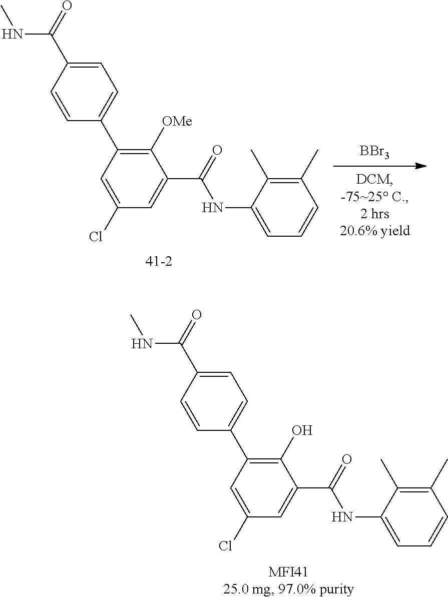

To a solution of compound 41-2 (500 mg, 1.18 mmol, 1.00 eq) in DCM (10.0 mL) was added BBr₃ (296 mg, 1.18 mmol, 114 μL, 1.00 eq) at −75° C. The mixture was stirred at 25° C. for 2 hrs. LCMS (EC14467-15-P1A1, product: RT=0.467 min) showed compound 41-2 was consumed completely and 58.3% of MFI41 was detected. The reaction mixture was quenched by addition H₂O 10.0 mL dropwise at 25° C. The mixture was extracted with the solution (DCM MeOH=5:1, 20.0 mL*2). The combined organic layers were washed with brine 30.0 mL and dried over Na₂SO₄, filtered and concentrated under reduced pressure to give a residue. The residue was purified by prep-HPLC (neutral condition) to give MFI41 (100 mg, 245 mol, 20.6% yield, 97.2% purity) as a pink solid.

LCMS: EC14467-15-P1A1, product: RT=2.243 mins, m/z=409.3 [M+H]⁺

LCMS: EC14467-28-P1A1, product: RT=2.173 mins, m/z=409.1 [M+H]⁺

HPLC: EC14467-28-P1H2, product: RT=3.293 mins, 97.2% purity

¹H NMR: EC14467-28-P1N1, (400 MHz, DMSO-d₄)

δ 13.38 (s, 1H), 10.58 (s, 1H), 8.49 (d, J=4.6 Hz, 1H), 8.26 (d, J=2.3 Hz, 1H), 7.89 (d, J=8.4 Hz, 1H), 7.71-7.61 (m, 1H), 7.18-7.12 (m, 1H), 2.81 (d, J=4.4 Hz, 1H), 2.30 (s, 1H), 2.33-2.27 (m, 1H), 2.12 (s, 1H)

### Preparation of Compound 43-2

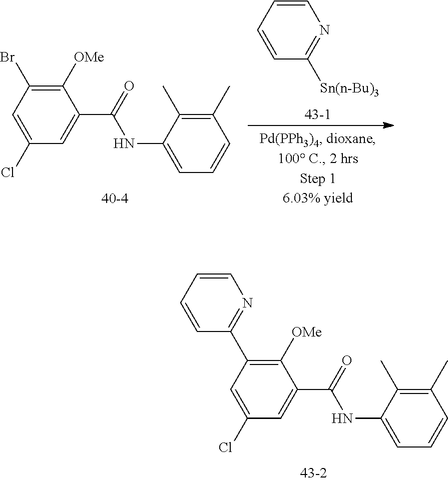

To a solution of compound 40-4 (2.00 g, 5.43 mmol, 1.00 eq) in dioxane (20.0 mL) was added Pd(PPh₃)₄ (1.25 g, 1.09 mmol, 0.20 eq) and compound 43-1 (2.00 g, 5.43 mmol, 1.00 eq) and the mixture was degassed and purged with N₂ for 3 times. The mixture was stirred at 100° C. for 2 hrs. LCMS (EC14467-50-P1A2, product: RT=0.455 min, SM: RT=0.586) showed 31.2% of compound 40-4 was remained and 11.3% of compound 43-2 was detected. The reaction mixture was filtered and concentrated under reduced pressure to give a residue. The residue was purified by column chromatography (SiO₂, Petroleum ether:EtOAc=30:1 to 10:1) to give compound 43-2 (250 mg, 327 mol, 6.03% yield, 48.0% purity) as a black brown solid.

LCMS: EC14467-50-P1A2, product: RT=0.373 min, m/z=367.0 [M+H]⁺

### Preparation of Compound MFI43

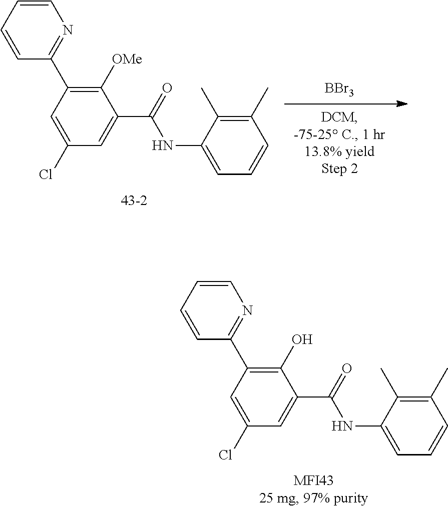

To a solution of compound 43-2 (200 mg, 545 mol, 1.00 eq) in DCM (2.00 mL) was added BBr₃ (137 mg, 545 mol, 1.00 eq) at −75° C. The mixture was stirred at 25° C. for 1 hr. LCMS (EC14467-62-P1A1, product: RT=2.956 mins) showed compound 43-2 was consumed completely and 53.1% of MFI43 was detected. The reaction mixture was quenched by addition H₂O 10.0 mL dropwise at 25° C. The mixture was extracted with the solution (DCM:MeOH=5:1, 20.0 mL*2). The combined organic layers were washed with brine 30.0 mL and dried over Na₂SO₄, filtered and concentrated under reduced pressure to give a residue. The crude product was purified by reversed-phase HPLC (neutral condition) to give MFI43 (60.0 mg, 75.2 mol, 13.8% yield, 44.2% purity) as a yellow solid.

LCMS: EC14467-62-P1A1, product: RT=2.956 mins, m/z=353.1 [M+H]⁺

LCMS: EC14467-62-P1A3, product: RT=2.373 mins, m/z=373.1 [M+H]⁺

HPLC: EC14467-62-P1H3, product: RT=2.849 mins, 97.3% purity

¹H NMR: EC14467-62-P1N1, (400 MHz, MEOH-d4)

δ 8.67-8.61 (m, 1H), 8.29-8.24 (m, 1H), 8.22 (d, J=2.6 Hz, 1H), 8.12 (d, J=2.6 Hz, 1H), 8.11-8.07 (m, 1H), 7.68-7.64 (m, 1H), 7.55-7.50 (m, 1H), 7.16-7.09 (m, 1H), 7.08-7.03 (m, 1H), 2.37-2.33 (m, 3H), 2.29 (s, 3H).

### Preparation of Compound 44-2

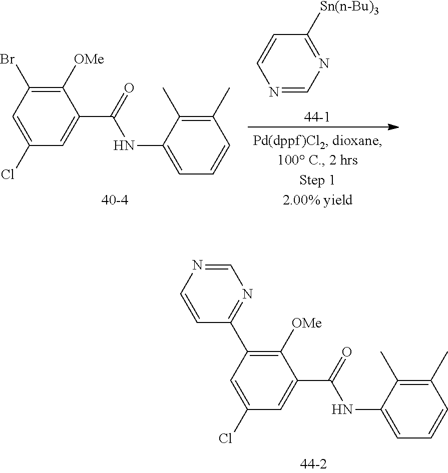

To a solution of compound 40-4 (1.20 g, 3.26 mmol, 1.50 eq) in dioxane (2.00 mL) was added CuI (206 mg, 1.09 mmol, 0.50 eq) and Pd(dppf)Cl₂ (318 mg, 434 mol, 0.20 eq) and compound 44-1 (801 mg, 2.17 mmol, 1.00 eq) and the mixture was degassed and purged with N₂ for 3 times. The mixture was stirred at 100° C. for 2 hrs. TLC (Petroleum ether:EtOAc=3:1, compound 40-4 R_{f}=0.80, compound 44-2 R_{f}=0.50) indicated compound 40-4 was consumed completely and a major new spot with larger polarity were detected. The reaction mixture was filtered and concentrated under reduced pressure to give a residue. The crude product was purified by reversed-phase HPLC (neutral condition) to give compound 44-2 (250 mg, 43.5 mol, 2.00% yield) as a yellow solid.

### Preparation of MFI44

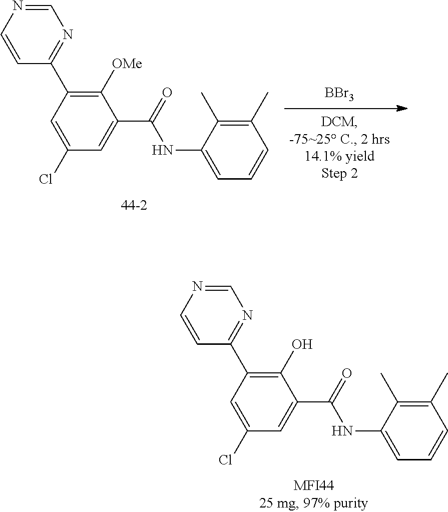

To a solution of compound 44-2 (200 mg, 544 mol, 1.00 eq) in DCM (5.00 mL) was added BBr₃ (204 mg, 816 mol, 1.50 eq) at −75° C. The mixture was stirred at 25° C. for 2 hrs. TLC (Petroleum ether:EtOAc=3:1, compound 44-2 R_{f}=0.40, MFI44 R_{f}=0.30) indicated 0% of compound 44-2 was remained, and one major new spot with larger polarity was detected. The reaction mixture was quenched by addition H₂O 10.0 mL dropwise at 25° C. The mixture was extracted with the solution (DCM:MeOH=5:1, 20.0 mL*2). The combined organic layers were washed with brine 30.0 mL and dried over Na₂SO₄, filtered and concentrated under reduced pressure to give a residue. The crude product was purified by reversed-phase HPLC (neutral condition) to give MFI44 (30.0 mg, 81.5 mol, 96.1% purity, 15.0% yield) as a yellow solid.

LCMS: EC14467-65-P1A, product: RT=2.213 mins, m/z=354.0 [M+H]⁺

HPLC: EC14467-65-P1H, product: RT=2.607 mins, 96.1% purity

1H NMR: EC14467-65-P1N (400 MHz, DMSO-d6)

δ 10.51-10.39 (m, 1H), 9.45-9.29 (m, 1H), 9.16-8.88 (m, 1H), 8.36 (s, 2H), 8.23 (br s, 1H), 7.42-7.34 (m, 1H), 7.18-7.06 (m, 2H), 2.33-2.27 (m, 3H), 2.17 (s, 3H)

### Preparation of Compound 2

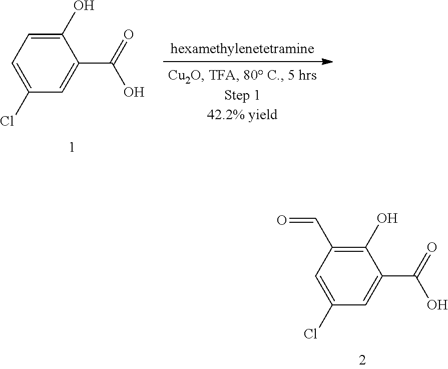 [SMILES: O=C(O)c1cc(Cl)ccc1O ; "hexamethylenetetramine" ; O=Cc1cc(Cl)cc(C(=O)O)c1O]

To a solution of compound 1 (20.0 g, 115 mmol, 1.00 eq) in TFA (200 mL) was added Cu₂O (16.6 g, 116 mmol, 1.00 eq) and hexamethylenetetramine (32.5 g, 231 mmol, 2.00 eq). The mixture was stirred at 80° C. for 5 hrs. LCMS (EC14486-3-P1A1, product: RT=0.551 min) showed compound 1 was consumed completely and 47.3% of compound 2 was detected. The reaction mixture was quenched by addition HCl (3 M, 200 mL) at 20° C., and concentrated under reduced pressure to give a residue. The crude product was purified by reversed-phase HPLC (5.00%-30.0% 45 min; 0.10% FA condition) to give compound 2 (10.9 g, 49.9 mmol, 42.2% yield) as a yellow solid.

LCMS: EC14486-3-P1A1, product: RT=0.551 min, m/z=202.0 [M+H]⁺

### Preparation of Compound 3

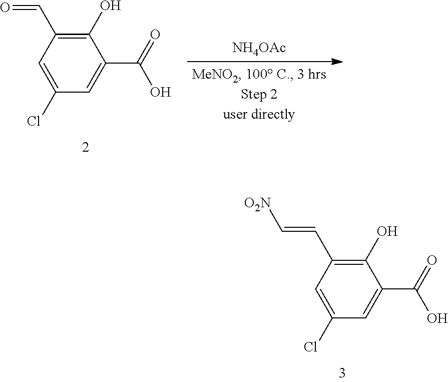 [SMILES: O=Cc1cc(Cl)cc(C(=O)O)c1O ; O=C(O)c1cc(Cl)cc(/C=C/[N+](=O)[O-])c1O ; "NH4OAc"]

To a solution of compound 2 (5.00 g, 24.9 mmol, 1.00 eq) in MeNO₂ (20.0 mL) was added NH₄OAc (8.40, 74.7 mmol, 3.00 eq). The mixture was stirred at 100° C. for 3 hrs. TLC (Petroleum ether:EtOAc=2:1, compound 2 R_{f}=0.20, compound 3 R_{f}=0.40) indicated compound 2 was consumed completely, and one major new spot with lower polarity was detected. The reaction mixture was filtered and concentrated under reduced pressure to give compound 3 (6.07 g, 30.8 mmol, crude) as a yellow solid.

### Preparation of Compound 4

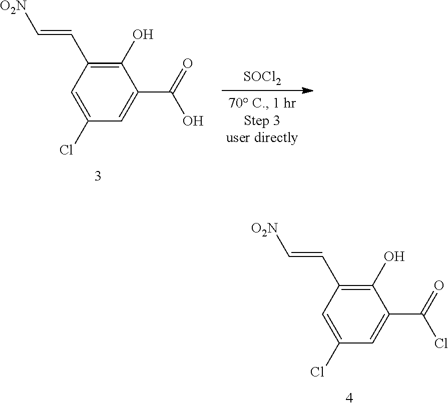

A mixture of compound 3 (6.07 g, 30.8 mmol, 1.00 eq) in SOCl₂ (50 mL) was degassed and purged with N₂ for 3 times, and then the mixture was stirred at 70° C. for 1 hr under N₂ atmosphere. TLC (Petroleum ether:EtOAc=3:1, compound 3 R_{f}=0.10, compound 4 R_{f}=0.30) indicated compound 3 was consumed completely, and one major new spot with lower polarity was detected. The reaction mixture was filtered and concentrated under reduced pressure to give compound 4 (6.53 g, 31.5 mmol, crude) as a yellow oil.

### Preparation of Compound 5

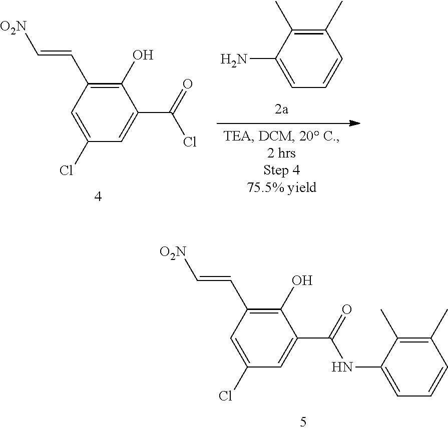

To a solution of compound 2a (4.58 g, 37.8 mmol, 1.20 eq) and TEA (6.35 g, 63.0 mmol, 2.00 eq) in DCM (200 mL) was added compound 4 (6.53 g, 31.5 mmol, 1.00 eq). The mixture was stirred at 20° C. for 2 hrs. LCMS (EC14486-56-P1L1, product: RT=0.429 min, SM: RT=0.394 min) showed compound 4 was consumed completely and 78.9% of compound 5 was detected. The reaction mixture was filtered and concentrated under reduced pressure to give compound 5 (1.20 g, 3.46 mmol, crude) as a yellow solid.

LCMS: EC14486-56-P1L1, product: RT=0.429 min, m/z=346.9 [M+H]⁺

### Preparation of Compound 6

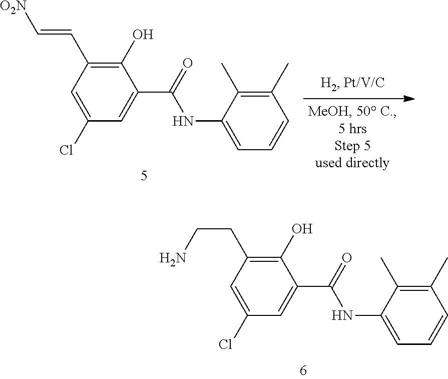 [SMILES: Cc1cccc(NC(=O)c2cc(Cl)cc(/C=C/[N+](=O)[O-])c2O)c1C ; Cc1cccc(NC(=O)c2cc(Cl)cc(CCN)c2O)c1C]

To a solution of compound 5 (1.20 g, 3.46 mmol, 1.00 eq) in MeOH (30.0 mL) was added Pt/V/C (1.70 g, 69.2 mol, 1.00% purity, 0.02 eq) under Ar₂ atmosphere. The suspension was degassed and purged with H2 for 3 times. The mixture was stirred under H2 (50 Psi) at 50° C. for 5 hrs. LCMS (EC14486-79-P1A2, product: RT=1.069 mins) showed compound 5 was consumed completely and 18.2% of compound 6 was detected. The reaction mixture was filtered and concentrated under reduced pressure to give compound 6 (30.0 mg, 941 mol, 27.2% yield) as a white solid.

LCMS: EC14486-79-P1A2, product: RT=1.069 mins, m/z=319.1 [M+H]⁺

### Preparation of MFI-COV1

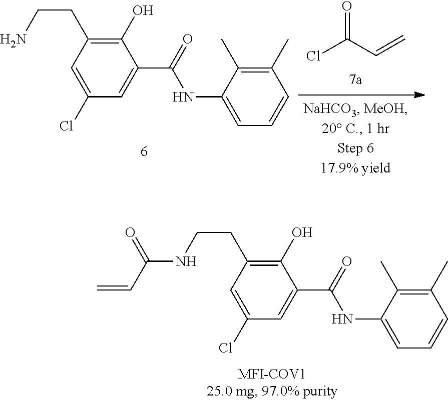

A mixture of compound 6 (20.0 mg, 62.7 mol, 1.00 eq), compound 7a (11.3 mg, 125 mol, 2.00 eq), NaHCO₃ (10.5 mg, 125 mol, 2.00 eq), in H₂O (2.00 mL) and MeOH (1.00 mL) was degassed and purged with N₂ for 3 times, and then the mixture was stirred at 20° C. for 1 hr under N₂ atmosphere. LCMS (EC14486-71-P1A1, product: RT=0.346 min, SM: RT=0.392 min) showed 2.47% of compound 6 was remained and 39.7% of MFI-COV1 was detected. The reaction mixture was filtered and concentrated under reduced pressure to give a residue. The crude product was purified by reversed-phase HPLC (neutral condition) to obtain MFI-COV1 (4.68 mg, 12.4 mol, 17.9% yield, 98.7% purity) as a white solid.

Statistical analysis. The results are presented as mean±SEM with a minimum of three replicates unless otherwise specified. Statistical analyses were performed by Student's t-tests or one-way or two-way ANOVA using Prism 8.1 (GraphPad software). When the overall ANOVA revealed a significant effect, the data were further analyzed with the Dunnett, Sidak or Tukey post hoc test to determine specific group differences. P values indicated on the graphs: * p<0.05, ** p<0.01, ***p<0.001, ****p<0.0001.

A small molecule inhibitor of mitochondrial fusion via mitofusins. Inter-molecular interactions of the HR2 domains of MFNs from adjacent mitochondria mediate MFNs oligomerization. It was therefore posited that a small molecule capable of inhibiting such inter-molecular HR2-HR2 interactions could act as an inhibitor of MFNs oligomerization and subsequently mitochondrial fusion. Structural analysis can be employed for the development a pharmacophore model, which is used in an in silico small molecule screening to identify small molecules that activate mitofusins. Previously a helical peptide from the MFN2 HR1 residues: 398-418 was found to inhibit mitochondrial fusion in cells by inhibiting HR2-HR2 inter-molecular interactions and subsequently MFNs oligomerization. This prompted visual inspection of the interactions of the HR1-residues 398-418 with the HR2 residues to gain structural insights for small molecule mimicry. Specifically, hydrophobic interactions were observed between the HR1-residues: Leu408, Ala412 and the HR2-residues: Leu692, Val688, and a possible hydrogen bond between the HR1-amino acid: Tyr415 and HR2-amino acid: Ser685. Of note, these HR1 residues are conserved among different species of MFN2 and MFN1. Hence, it was envisioned that a small molecule capable of recapitulating the interactions of the aforementioned HR1-amino acids would compete with inter-molecular HR2-HR2 interactions and inhibit MFNs oligomerization. Accordingly, to identify such a small molecule a strategy was adopted including virtual library preparation, pharmacophore screen with Phase, selection of top-ranked hits, interaction analysis and molecular property-based selection, and testing of selected hits experimentally. An in silico pharmacophore model was generated that screens small molecules to mimic specifically the side chains of the HR1 residues: Leu408, Ala412 and Tyr415 and bind to the corresponding HR2 residues. The pharmacophore model includes two hydrophobic interactions, an aromatic ring and a hydrogen bond donor/acceptor. Next the in silico library of 13.8×10⁶ commercially available small molecules was screened using the strategy described above. Likewise a set of 21 putative Mitochondrial Fusion Inhibitors (MFIs) was selected for experimental validation based on their fit to the pharmacophore model and molecular diversity of their scaffolds.

Selected hits were screened for their capacity to inhibit mitochondrial fusion in cells by monitoring mitochondrial morphology and using mitochondrial aspect ratio as a readout. Strikingly, MFI8 reduced significantly mitochondrial aspect ratio and emerged as the most effective compound in inhibiting mitochondrial fusion and subsequently promoting mitochondrial fission. Titration of MFI8 showed a concentration-dependent reduction of the mitochondrial aspect ratio. MFI8 has a small structure but possesses functional groups that could fulfill the 4 criteria of the pharmacophore model used for the in silico screen. Thus, the phenolic ring could participate in pi-stacking interactions and act as a hydrogen bond donor/acceptor, as the side chain of Tyr415, while the dimethyl-substituted phenyl ring could mimic the hydrophobic interactions of Leu408 and Ala412. To further validate that MFI8 meets the 4 criteria of the pharmacophore model hypothesis, structure activity relationships around the MFI8 scaffold were investigated. A series of MFI8 analogues were generated and evaluated for their capacity to promote mitochondrial fragmentation in cells. Substitution of the chlorine with hydrogen in the aromatic ring of MFI22 significantly reduced the capacity of the compound to promote mitochondrial fragmentation, suggesting that loss of chlorine affects the electron density of the aromatic ring and impairs its interaction. Loss of the methyl group in the aliphatic chain that connects the two phenyl rings had a minimal effect on the activity of MFI23. Furthermore, loss of each methyl group in the phenyl ring of MFI8 was not tolerated as MFI24 and MFI25 did not reduce mitochondrial aspect ratio, indicating the importance of those two methyl groups in mediating hydrophobic contacts for MFI8. Importantly, loss of the hydroxyl group of the phenolic ring was detrimental for the activity of MFI26, indicating that MFI8 forms a crucial hydrogen bond with the HR2 domain. Taken together, these data indicated that MFI8 possesses functional groups that meet the criteria of the pharmacophore model and specifically interact with mitofusins.

Next it was investigated whether MFI8 inhibited mitochondrial fusion in a MFNs dependent manner using mitochondrial aspect ratio as a readout. MFI8 was still capable of reducing mitochondrial aspect ratio when either MFN1 or MFN2 was knocked out. In contrast, double knockout of MFN1 and MFN2 completely abolished MFI8 from reducing mitochondrial aspect ratio. Collectively, these data indicate that MFI8 can promote mitochondrial fission by inhibiting mitochondrial fusion and interfering with the formation of either homotypic or heterotypic MFNs complexes. Importantly, MFI8 reduced mitochondrial aspect ratio even when it was co-treated with MASM7 in MEFs. Such a result supports the idea that MFI8 operates on the tethering permissive structure of MFNs and inhibits MFNs oligomerization by reducing the H1R2-HR2 inter-molecular interactions.

Mitochondrial fusion positively correlates with mitochondrial respiration and membrane potential. Furthermore, inhibition of mitochondrial fusion decreases mitochondrial membrane potential and subsequently respiration. MASM7 increases mitochondrial functionality such as mitochondrial respiration and membrane potential. MFI8 decreases mitochondrial functionality such as mitochondrial respiration and membrane potential. A series of mutagenesis experiments in vitro and in cellulo supported the observation that both MASM7 and MFI8 interact specifically with the H1R2 domain of MFN2, albeit at different binding sites. Additional experiments from isolated mitochondria and in cells were used to demonstrate that both MASM7 and MFI8 modulate the conformational plasticity of MFN2 and its capacity to form oligomers. MASM7 promotes oligomerization of MFN2 by promoting the pro-tethering conformation of MFN2, while MFI8 directly inhibits the mitofusin oligomerization.

Inhibition of mitochondrial fusion promotes minority mitochondrial outer membrane permeabilization, and subsequently caspase 3/7 activation, albeit at sub-lethal levels. Aberrant mitochondrial fragmentation promotes minority MOMP and sub-lethal caspase 3/7 activation. Experiments have connected mitochondrial shape with mitochondrial membrane integrity and caspase 3/7 activation. Further experiments showed that MFI8 sensitizes cells to BV6 (SMAC mimetic) treatment in a mitofusin and APAF-1 dependent manner. Inhibition of mitochondrial fusion promotes minority MOMP and activates caspases 3/7, which can be used in combination with chemotherapeutic agents that act downstream of MOMP, such as Smac mimetics (e.g. BV6), to promote synergistic cell death in cells.

MFI8 binds to the HR2 domain of MFN2. To confirm that MASM7 and MFI8 directly interact with the HR2 domain, for the first time an ¹⁵N-labeled MFN2-HR2 domain (residues 678-757) was prepared and heteronuclear single quantum coherence (HSQC) spectra with or without MASM7 or MFI8 was recorded. HSQC NMR spectra of the recombinant HR2 domain showed evidence of a folded conformation of HR2. Titration of MASM7 induced peak broadening and shifting of select cross peaks of HR2 residues in the HSQC spectra, demonstrating direct binding to the HR2 domain. Similar cross peaks broadened and shifted upon titration of the 367-384Gly peptide, indicating that both MASM7 and 367-384Gly interact with similar HR2 residues. To further confirm that the interaction of MASM7 is specific to the expected binding region of HR2, a double mutant of HR2, D725A/L727A, was generated that should disrupt binding of MASM7 based on its proposed interaction with the HR2 domain. Titration of MASM7 to an 15N-labeled MFN2-HR2 D725A/L727A mutant under the same conditions showed lack of peak shifts or peak broadening indicating lack of MASM7 binding to the MFN2-HR2 D725A/L727A mutant.

Titration of MFI8 to the 15N-labeled MFN2-HR2 revealed peak broadening and shifting of select cross peaks of HR2 residues in the HSQC spectra, showing that MFI8 directly interacts with the HR2 domain of MFN2. Additionally, similar cross peaks shifted upon titration of the 398-418Gly peptide, indicating interaction with similar HR2 residues for both MFI8 and 398-418Gly. Notably, titration of MFI8 to ¹⁵N-labeled MFN2-HR2 S685A mutant under the same conditions showed lack of peak shifts or peak broadening indicating lack of MFI8 binding to the MFN2-HR2 S685A mutant.

To further confirm that MASM7 and MFI8 directly interact with the HR2 domain, MFN2-HR2 domain (residues 678-757) was produced and evaluated for their interaction in comparison with 367-384Gly and 398-418Gly peptides using microscale thermophoresis (MST). MASM7 and MFI8 demonstrated direct binding to the HR2 domain of MFN2 with Kds in the low micromolar range and that was comparable to the binding interactions of 367-384Gly and 398-418Gly peptides, respectively. Furthermore, MASM23 and MFI23 demonstrated direct binding to the HR2 domain of MFN2, in line with our previous results that showed that these small molecules are capable of increasing or decreasing the mitochondrial aspect ratio, respectively. In contrast, MASM19, MASM21, MASM22, MFI22, MFI25 and MFI26 did not demonstrate measurable binding to the HR2 domain of MFN2, in agreement with the inability or weak activity of these compounds to promote mitochondrial fusion or fision, respectively.

Next it was determined whether MFI8 also interacted with the HR2 domain of MFN2 in cells. MASM7 markedly increased mitochondrial aspect ratio in MFN1/MFN2 DKO MEFs when reconstituted with WT MFN2, but not when reconstituted with D725A/L727A or L727A MFN2 mutants, underscoring that MASM7 specifically targets the HR2 domain of MFN2 in cells. Likewise, MFI8 significantly decreased mitochondrial aspect ratio in MFN1/MFN2 DKO MEFs when reconstituted with WT MFN2, but not when reconstituted with S685A or L692A MFN2 mutants, indicating Ing that MFI8 interacts specifically with the HR2 domain of MFN2 in cells. Collectively, MST, NMR and mitochondrial morphology data supported that MASM7 and MFI8 specifically interact with the HR2 domain as predicted from the pharmacophore models.

MASM7 and MFI8 modulates MFN conformation and complexes. Next, it was investigated whether MASM7 and MFI8 can modulate MFN2 oligomerization in its native membrane environment. Thus, isolated mitochondria from MEFs were treated with MASM7 or MFI8 and the capacity of MFN2 to form oligomers was monitored using blue native gel electrophoresis (BN-PAGE). MFN2 migrated as a dimer in the absence of GTP, while incubation with GTP promoted higher order oligomers ~450 kD. Indeed, treatment of MASM7 in isolated mitochondria increased the ratio of higher order oligomers ~450 kD to dimers upon GTP binding, whereas MFI8 reduced the ratio.

In contrast, MFI8 alone had no effect on the Tₘ of MFN2 (Veh: Tm=42.9±0.8° C. vs MFI8: Tm=43.7±1.1° C.). Given that MFI8 binds in vitro and in cells on the HR2 domain of MFN2, reduces the ratio of higher order MFN2 oligomers to dimers and inhibits mitochondrial fusion in a MFN dependent manner, it was speculated that MFI8 engages better with the pro-tethering conformation of MFN2 in which the H1R2 domain is exposed in the cytoplasm. To test this hypothesis cells were co-treated with MFI8 and MASM7, where the latter compound was found promote the pro-tethering conformation and increase the Tm of MFN2. Strikingly, MFI8 revoked MASM7-induced MFN2 stabilization (Veh: Tm=42.9±0.8° C. vs MFI8+MASM7: Tm=44.05±2.6° C.). This result is consistent with previous experiments showing that MFI8 can still promote mitochondrial fragmentation even when co-treated with MASM7. Besides target engagement, MASM7-induced MFN2 stabilization can also be attributed to the increased complexation of MFN2 with other proteins, presumably MFN1 and MFN2 as part of their functional oligomerization to mediate mitochondrial fusion. Thus, the effect of MFI8 treatment on MASM7-induced MFN2 stabilization indicates that MFI8 destabilizes homotypic (MFN2-MFN2) or heterotypic (MFN1-MFN2) complex formation.

It is noteworthy that neither MASM7 nor MFI8 altered MFN1 and MFN2 gene expression and their corresponding protein levels. Furthermore, neither MASM7 nor MFI8 altered Tomm20 protein levels, mitotracker green intensity and mitochondrial to nuclear DNA ratio, suggesting that none of the compounds altered mitochondrial biomass. In line with this result, no alteration in the gene expression levels of mitochondrial biogenesis markers was detected upon MASM7 or MFI8 treatment.

Modulation of mitofusins activity alters mitochondrial respiration and functionality. Previous reports showed that knockdown of MFN2 or knockout of both MFNs reduced respiration, suggesting that mitofusins positively regulate mitochondrial respiration. Given that MASM7 positively and MFI8 negatively regulate MFNs' fusogenic activity, it was examined whether they can affect mitochondrial respiration. Strikingly, MASM7 increased basal and maximal respiration and mitochondrial ATP production in WT but not in MFN1/MFN2 DKO MEFs. On the other hand, MFI8 reduced respiration and mitochondrial ATP production in WT but not in MFN1/MFN2 DKO MEFs. Importantly, MFI8 did not alter the ratio of state III/state II respiration of isolated mitochondria, indicating that MFI8 does not impact directly the electron transport chain (ETC) or is an unspecific uncoupler to isolated mitochondria but it rather reduces mitochondrial respiration by modulating mitochondrial dynamics. These results support the idea that the fusogenic activity of MFNs positively correlates with mitochondrial respiration.

To further support this idea WT MFN2 or β-Galactosidase (βGal) was reconstituted in MFN1/MFN2 DKO MEFs and the mitochondrial membrane potential was evaluated using TMRE staining as a readout. Interestingly, cells that expressed WT MFN2 possessed a higher mitochondrial membrane potential compared to cells that expressed βGal. Consistently, MASM7 concentration responsively increased mitochondrial membrane potential in WT MEFs. In line with this result, MASM7 significantly increased mitochondrial membrane potential of MFN1/MFN2 DKO MEFs when reconstituted with WT MFN2 and to a lesser extent when reconstituted with L727A MFN2. Importantly, MASM7-induced increase in mitochondrial membrane potential was revoked when cells were co-treated with myxothiazol or rotenone, indicating that the increase in the membrane potential upon MASM7 treatment is derived from an increased activity of the ETC. On the other hand, MFI8 concentration responsively decreased mitochondrial membrane potential in WT MEFs. It is noteworthy that MFI8 significantly decreased mitochondrial membrane potential of MFN1/MFN2 DKO MEFs when reconstituted with WT MFN2 but not when reconstituted with L692A MFN2. Strikingly, MFI8 reduced the gene expression of several nuclear-encoded subunits of the respiratory complexes as revealed by RNA-seq analysis, while MASM7 altered expression of selected genes of the respiratory complexes rather than inducing a consistent trend. Such result is in line with the idea that MFI8 promotes mitochondrial dysfunction and highlights that alterations in mitochondrial dynamics can affect gene transcription.

Next, it was determined whether such alterations can have an impact on the metabolites of the TCA cycle. Surprisingly, MASM7 had no significant effect in the majority of the metabolites of the TCA cycle. On the contrary, MFI8 markedly reduced several metabolites such as malate, oxaloacetate, and α-ketoglutarate. Consistently, MFI8 decreased the total NAD⁺/NADH ratio, which is consistent with reduced oxidative capacity. In contrast, MASM7 had no effect on total NAD⁺/NADH ratio. Collectively, these data indicate that MFNs act as regulators of mitochondrial functionality and that mitochondrial fusion positively correlates with mitochondrial functionality.

Inhibition of mitofusin activity induces minority MOMP and primes cells for cell death. Previous reports highlighted the importance of mitochondrial shape and function in the execution and sensitivity of the cells to various types of cell death, such as apoptosis. Remarkably, MFI8 concentration-responsively increased caspase-3/7 activity, a key component of apoptotic signaling, whereas MASM7 had no effect on it. Furthermore, an analogue of MFI8 that is unable to inhibit MFNs' fusogenic activity and induce aberrant mitochondrial fragmentation, MFI22, did not increase caspase-3/7 activity. Consistently, deletion of MFN1/MFN2 also impaired the capacity of MFI8 to increase caspase-3/7 activity. Taken together these data indicate that MFI8 induces caspase 3/7 activation in a MFNs dependent manner, and such phenotype is associated with mitochondrial fragmentation. Deletion of APAF-1 was detrimental for the capacity of MFI8 to increase caspase 3/7 activity, indicating that apoptosome formation is crucial for the MFI8-induced caspase 3/7 activation. Furthermore, cytosolic and mitochondrial fractions were also analyzed upon treatment with MFI8 and it was found that cytochrome c was released to the cytosol, albeit at modest levels and in a mitofusin dependent manner. Interestingly, MFI8 did not increase the percentage of dead cells upon caspase 3/7 activation. Consistently, neither MFI8 nor MASM7 decreased cellular viability over the course of 72 hours.

Since cytochrome c release and caspase-3/7 activation was detected upon MFI8 treatment, it was posited that inhibition of mitochondrial fusion by MFI8 could induce mitochondrial outer membrane permeabilization (MOMP). Using an established live cell imaging assay in U2OS cells that expressed fluorescent cytosolic (CytoGFP) and mitochondrial (MitoCherry) probes, MOMP was quantified. Notably, MFI8 induced MOMP, albeit at modest levels, Accordingly, MFI8 but not MASM7 induced caspase-3/7 activation in U2OS cells. Of note, MFI8 also inhibited mitochondrial fusion in U2OS cells. Taken all together these data indicate that MFI8 induced minority MOMP and subsequent activation of caspases 3/7, albeit at sub-lethal levels. Previously, it was reported that minority MOMP induces genomic instability. Hence, the capacity of MFI8 in inducing DNA damage using γH2AX as a readout was evaluated. Interestingly, MFI8 increased γH2AX foci in WT MEFs but not in MFN1/MFN2 DKO MEFs, demonstrating that MFI8 induces DNA damage in a MFNs dependent manner. Of note, MASM7 did not induce DNA damage in any of the cell lines. Consistently, MFI8 up-regulated several genes that are involved in DNA damage response in MEFs. Moreover, MFI8 induced DNA damage in U2OS cells. Importantly, co-treatment of a pan-caspase inhibitor, Q-VD-OPh, with MFI8 abolished the capacity of the latter to induce DNA damage in U2OS.

It was further investigated if induction of minority MOMP by MFI8 can be used to enhance the capacity of another pro-apopotic agent to induce cell death. Thus, BV6, a bivalent SMAC mimetic that induces caspase-dependent cell death predominantly via XIAP inhibition was used. Notably, deletion of MFNs sensitized cells to BV6 treatment. Consistently, MFI8 potentiated the capacity of BV6 to induce cell death. The effect of MFI8 was specific to MFN1/2 inhibition as MFI22 did not sensitize cells to BV6 treatment. Importantly, deletion of MFN1/2 and APAF abolished the capacity of MFI8 to sensitize cells to BV6 treatment. Collectively, these results demonstrate that inhibition of MFNs' fusogenic activity induces minority MOMP and sub-lethal caspase-3/7 activation, which leads to DNA damage and can sensitize cells to apoptotic cell death in combination with a SMAC-mimetic. Further experiments showed that MFI8 synergizes with SMAC mimetic BV6 in THP-1 cells. More specifically, it was observed that MFI8 and SMAC mimetic BV6 synergistically induce cell death in THP-1 and U2OS cells.

It was also discovered that MFI8 synergizes with BH3 mimetic (MCL-1 inhibitor) AMG-176 in MOLM13 cells. Results showed that MFI8 sensitizes cells to caspase activation in response to BH3 mimetic (MCL-1 inhibitor) AMG-176 treatment. An increase in caspase 3/7 activation was observed in MOLM13 cells treated with increasing concentrations of AMG-176 and MFI8 (n=3, mean±SD).

These studies demonstrated direct binding of the mitofusin activator MASM7 and mitofusin inhibitor MFI8 to the recombinant HR2 domain of MFN2 and to intact MFN2 localized at mitochondria in cells. Binding and activity data come in agreement with our pharmacophore models where MASM7 and MFI8 were designed to interact with the H1R2 residues based on the side chains of HR1 residues. MASM7 promotes the pro-tethering conformation of MFNs to enable mitochondrial fusion, whereas MFI8 impedes mitochondrial fusion by directly interfering with the tethering premising structure of MFNs. MASM7 and MFI8 were found to increase or decrease, respectively, the GTP-dependent MFN2 higher-order oligomers, demonstrating these small molecules can modulate the levels of pro-fusion oligomers, and therefore the extent of fusion among mitochondria. These data also support that the activity of MASM7 and MFI8 through their interactions with the HR2 domain of MFN1/2 is compatible with the proposed GTP-mediated dimerization mechanism of the GTPase domains of MFNs. The data indicated that MASM7 can activate both MFN2 and MFN1, while MFI8 can inhibit both MFN2 and MIFN1. This can be attributed to the high sequence homology between MFN1 and MFN2, and the conservation of the residues that are located in the binding region of each small molecule between MIFN1 and MFN2.

MFNs modulators reported here allow temporal manipulation of the fusogenic activity of MFNs in a reversible fashion. This is in contrast to other small molecules that have been reported such as the drug Leflunomide, which alters MFNs protein levels through loss of pyrimidine synthesis and are likely to affect non-fusogenic functions of MFNs. Importantly, the rational discovery of MASM7 and MFI8 enables development of novel therapeutics for disorders/syndromes where impaired mitochondrial dynamics contributes to pathogenesis. Defective MFN2 mutants have been associated with development of Charcot-Marie-Tooth disease type 2A (CMT2A) and imbalances in mitochondrial dynamics have been linked to metabolic disorders such as type II diabetes, obesity, neurodegeneration, cancer and aging. Several mutations on the GTPase, HR1 and HR2 domain of the Mfn2 have been identified from patient samples and correlated with the development of the CMT2A disease. Interestingly, M376, a residue that was used for the development of the pharmacophore model for the discovery of the MFN2 activators, has been found to be mutated in patient samples to Val. Moreover, Leu724, a residue that is located in the binding region of the MFN2 activators, has been found to be mutated in patient samples to Pro. These patient mutations highlight the importance of those residues in HR1 and HR2 domains for the function of the protein. Notably, MFN2 activators have been shown to rescue mitochondrial defects of CMT2A in preclinical models raising the opportunity for the development of novel therapeutics.

MEFs were seeded on chamber slides (MatTek Corporation: 35 mm dishes, No. 1.5, 14 mm glass diameter) at ~70% confluence. Cells were treated with compounds. Then, cells were stained with MitoTracker Green (200 nM, Invitrogen) for 20 min at 37° C. After treatments, media was replaced with FluoroBrite™ DMEM (Invitrogen) supplemented with 10% FBS, 100 U ml⁻¹ penicillin/streptomycin, 2 mM L-glutamine and compound prior image acquisition. Images were taken with Leica SP5 inverted confocal microscope. Data were analyzed with Image J software. >200 mitochondria were measured per condition in mitochondrial aspect ratio. Measurement of mitochondrial aspect ratio (Mito AR). Images that depict stained mitochondria with mitotracker green were analyzed using FIJI software. Each mitochondrion was measured for its length and width. Then, the length was divided by the width for each mitochondrion to get its mitochondrial aspect ratio. >200 mitochondria were measured per treatment.

All references cited herein are incorporated by reference in their entireties.

It will be appreciated by persons skilled in the art that the invention described herein is not limited to what has been particularly shown and described. Rather, the scope of the invention is defined by the claims which follow. It should further be understood that the above description is only representative of illustrative examples of embodiments. The description has not attempted to exhaustively enumerate all possible variations. The alternate embodiments may not have been presented for a specific substituent of the compound, or a step of the method, and may result from a different combination of described substituent or step, or that other undescribed alternate embodiments may be available for a compound or method, is not to be considered a disclaimer of those alternate embodiments. It will be appreciated that many of those un-described embodiments are within the literal scope of the following claims, and others are equivalent.

1. A compound of formula I or a pharmaceutically acceptable salt thereof, 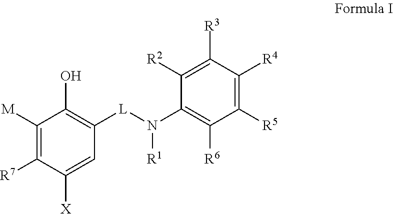
wherein
M is selected from hydrogen, deuterium, OC₁₋₆alkyl, SC₁₋₆alkyl, CN, OH, SH, COOH, halogen, NO₂, N(R^{m})₂, C(O)OR^{m}, C(O)N(R^{m})₂, C(O)C₁₋₆alkyl, haloC₁₋₆alkyl, haloC₁₋₆alkyleneO, C₁₋₆alkyl, hydroxyC₁₋₆alkyl, dihydroxyC₁₋₁₀alkyl, C₃₋₆cycloalkyl, C(═NC₁₋₆alkyl)C₁₋₆alkyl, OC(O)N(R^{m})₂, C(O)SR^{m}, OC₁₋₆alkyleneOC₁₋₆alkyl, OC₁₋₆alkyleneO-haloC₁₋₆alkyl, SC₁₋₆alkyleneOC₁₋₆alkyl, SC₁₋₆alkyleneSC₁₋₆alkyl, OC₁₋₆alkyleneSC₁₋₆alkyl, SC₁₋₆alkyleneO-haloC₁₋₆alkyl, SC₁₋₆alkyleneS-haloC₁₋₆alkyl, OC₁₋₆alkyleneS-haloC₁₋₆alkyl, C₁₋₆alkylene-CN, OC₁₋₆alkylene-CN, SC₁₋₆alkylene-CN, OC₁₋₆alkylene-N(R^{m})₂, C₂₋₆alkynyl, C₂₋₆alkenyl, SO₂N(R^{m})₂, NR^{m}SO₂C₁₋₆alkyl, C₁₋₆alkylSO₂ (sulfone), S(O)OH, C₁₋₆alkylS(O) (sulfoxide), nitroso, and C₁₋₆alkylOSO₂, or Ar,
Ar is a phenyl, naphthyl, 5-10-membered heteroaryl or 4-8 membered heterocyclyl, wherein the phenyl, naphthyl, heteroaryl or heterocycyl is optionally substituted with one more substituents selected from the group consisting of deuterium, OC₁₋₆alkyl, SC₁₋₆alkyl, CN, OH, SH, COOH, halogen, NO₂, N(R^{m})₂, C(O)OR^{m}, C(O)N(R^{m})₂, C(O)C₁₋₆alkyl, haloC₁₋₆alkyl, haloC₁₋₆alkyleneO, C₁₋₆alkyl, hydroxyC₁₋₆alkyl, dihydroxyC₁₋₁₀alkyl, C₃₋₆cycloalkyl, C(═NC₁₋₆alkyl)C₁₋₆alkyl, OC(O)N(R^{m})₂, C(O)SR^{m}, OC₁₋₆ alkyleneOC₁₋₆alkyl, OC₁₋₆alkyleneO-haloC₁₋₆alkyl, SC₁₋₆alkyleneOC₁₋₆alkyl, SC₁₋₆ alkyleneSC₁₋₆alkyl, OC₁₋₆alkyleneSC₁₋₆alkyl, SC₁₋₆alkyleneO-haloC₁₋₆alkyl, SC₁₋₆ alkyleneS-haloC₁₋₆alkyl, OC₁₋₆alkyleneS-haloC₁₋₆alkyl, C₁₋₆alkylene-CN, OC₁₋₆alkylene-CN, SC₁₋₆alkylene-CN, OC₁₋₆alkylene-N(R^{m})₂, C₂₋₆alkynyl, C₂₋₆alkenyl, SO₂N(R^{m})₂, NR^{m}SO₂C₁₋₆alkyl, C₁₋₆alkylSO₂ (sulfone), S(O)OH, C₁₋₆alkylS(O) (sulfoxide), nitroso, and C₁₋₆alkylOSO₂;
X is halogen;
L is C₁₋₃alkylene optionally substituted with an oxo (═O), SO, SO₂ or OC(═O);
R¹ is hydrogen, C₁₋₆alkyl or C(O)C₁₋₄alkyl,
R² and R³ are independently selected from the group consisting of hydrogen, OC₁₋₆alkyl, SC₁₋₆alkyl, C₁₋₄alkyl, CN, halogen, C₁₋₆alkylene-CN, OC₁₋₆alkylene-CN, haloC₁₋₆alkyl, SC₁₋₆alkylene-CN, C₂₋₆alkynyl, C₂₋₆alkenyl, and C₁₋₆alkylSO₂ (sulfone);
R⁴, R⁵, and R⁶ are independently selected from the group consisting of hydrogen, deuterium, OC₁₋₆alkyl, SC₁₋₆alkyl, CN, OH, SH, halogen, NO₂, N(R^{m})₂, C(O)OR^{m}, C(O)N(R^{m})₂, C(O)C₁₋₆alkyl, haloC₁₋₆alkyl, haloC₁₋₆alkyleneO, C₁₋₆alkyl, hydroxyC₁₋₆alkyl, dihydroxyC₁₋₁₀alkyl, C₃₋₆cycloalkyl, C(═NC₁₋₆alkyl)C₁₋₆alkyl, OC(O)N(R^{m})₂, C(O)SR^{m}, OC₁₋₆alkyleneOC₁₋₆alkyl, OC₁₋₆alkyleneO-haloC₁₋₆alkyl, SC₁₋₆alkyleneOC₁₋₆alkyl, SC₁₋₆alkyleneSC₁₋₆alkyl, OC₁₋₆alkyleneSC₁₋₆alkyl, SC₁₋₆alkyleneO-haloC₁₋₆alkyl, SC₁₋₆alkyleneS-haloC₁₋₆alkyl, OC₁₋₆alkyleneS-haloC₁₋₆alkyl, C₁₋₆alkylene-CN, OC₁₋₆ alkylene-CN, SC₁₋₆alkylene-CN, OC₁₋₆alkylene-N(R^{m})₂, C₂₋₆alkynyl, C₂₋₆alkenyl, SO₂N(R^{m})₂, NR^{m}SO₂C₁₋₆alkyl, C₁₋₆alkylSO₂ (sulfone), S(O)OH, C₁₋₆alkylS(O) (sulfoxide), nitroso, C₁₋₆alkylOSO₂;
R⁷ is selected from the group consisting of hydrogen, deuterium, OC₁₋₆alkyl, SC₁₋₆alkyl, CN, OH, SH, halogen, NO₂, N(R^{m})₂, C(O)OR^{m}, C(O)N(R^{m})₂, C(O)C₁₋₆alkyl, haloC₁₋₆alkyl, haloC₁₋₆alkyleneO, C₁₋₆alkyl, hydroxyC₁₋₆alkyl, dihydroxyC₁₋₁₀alkyl, C₃₋₆cycloalkyl, C(═NC₁₋₆alkyl)C₁₋₆alkyl, OC(O)N(R^{m})₂, C(O)SR^{m}, OC₁₋₆alkyleneOC₁₋₆ alkyl, OC₁₋₆alkyleneO-haloC₁₋₆alkyl, SC₁₋₆alkyleneOC₁₋₆alkyl, SC₁₋₆alkyleneSC₁₋₆alkyl, OC₁₋₆alkyleneSC₁₋₆alkyl, SC₁₋₆alkyleneO-haloC₁₋₆alkyl, SC₁₋₆alkyleneS-haloC₁₋₆alkyl, OC₁₋₆alkyleneS-haloC₁₋₆alkyl, C₁₋₆alkylene-CN, OC₁₋₆alkylene-CN, SC₁₋₆alkylene-CN, OC₁₋₆alkylene-N(R^{m})₂, C₂₋₆alkynyl, C₂₋₆alkenyl, SO₂N(R^{m})₂, NR^{m}SO₂C₁₋₆alkyl, C₁₋₆alkylSO₂ (sulfone), S(O)OH, C₁₋₆alkylS(O) (sulfoxide), nitroso, C₁₋₆alkylOSO₂, phenyl, naphthyl, 5-10-membered heteroaryl or 4-8 membered heterocyclyl, wherein the phenyl, naphthyl, heteroaryl or heterocycyl is optionally substituted with one more substituents selected from the group consisting of deuterium, OC₁₋₆alkyl, SC₁₋₆alkyl, CN, OH, SH, COOH, halogen, NO₂, N(R^{m})₂, C(O)OR^{m}, C(O)N(R^{m})₂, C(O)C₁₋₆alkyl, haloC₁₋₆alkyl, haloC₁₋₆alkyleneO, C₁₋₆alkyl, hydroxyC₁₋₆alkyl, dihydroxyC₁₋₁₀alkyl, C₃₋₆cycloalkyl, C(═NC₁₋₆alkyl)C₁₋₆alkyl, OC(O)N(R^{m})₂, C(O)SR^{m}, OC₁₋₆alkyleneOC₁₋₆alkyl, OC₁₋₆ alkyleneO-haloC₁₋₆alkyl, SC₁₋₆alkyleneOC₁₋₆alkyl, SC₁₋₆alkyleneSC₁₋₆alkyl, OC₁₋₆ alkyleneSC₁₋₆alkyl, SC₁₋₆alkyleneO-haloC₁₋₆alkyl, SC₁₋₆alkyleneS-haloC₁₋₆alkyl, OC₁₋₆alkyleneS-haloC₁₋₆alkyl, C₁₋₆alkylene-CN, OC₁₋₆alkylene-CN, SC₁₋₆alkylene-CN, OC₁₋₆ alkylene-N(R^{m})₂, C₂₋₆alkynyl, C₂₋₆alkenyl, SO₂N(R^{m})₂, NR^{m}SO₂C₁₋₆alkyl, C₁₋₆alkylSO₂ (sulfone), S(O)OH, C₁₋₆alkylS(O) (sulfoxide), nitroso, and C₁₋₆alkylOSO₂
optionally, two adjacent substituents of R², R³, R⁴, R⁵, and R⁶ link up to form, together with the phenyl they are attached to, a bicyclic ring, wherein the bicyclic ring is optionally substituted with one more substituents selected from the group consisting of deuterium, OC₁₋₆alkyl, SC₁₋₆alkyl, CN, OH, SH, COOH, halogen, NO₂, N(R^{m})₂, C(O)OR^{m}, C(O)N(R^{m})₂, C(O)C₁₋₆alkyl, haloC₁₋₆alkyl, haloC₁₋₆alkyleneO, C₁₋₆alkyl, hydroxyC₁₋₆alkyl, dihydroxyC₁₋₁₀alkyl, C₃₋₆cycloalkyl, C(═NC₁₋₆alkyl)C₁₋₆alkyl, OC(O)N(R^{m})₂, C(O)SR^{m}, OC₁₋₆alkyleneOC₁₋₆alkyl, OC₁₋₆alkyleneO-haloC₁₋₆alkyl, SC₁₋₆ alkyleneOC₁₋₆alkyl, SC₁₋₆alkyleneSC₁₋₆alkyl, OC₁₋₆alkyleneSC₁₋₆alkyl, SC₁₋₆alkyleneO-haloC₁₋₆alkyl, SC₁₋₆alkyleneS-haloC₁₋₆alkyl, OC₁₋₆alkyleneS-haloC₁₋₆alkyl, C₁₋₆alkylene-CN, OC₁₋₆alkylene-CN, SC₁₋₆alkylene-CN, OC₁₋₆alkylene-N(R^{m})₂, C₂₋₆alkynyl, C₂₋₆alkenyl, SO₂N(R^{m})₂, NR^{m}SO₂C₁₋₆alkyl, C₁₋₆alkylSO₂ (sulfone), S(O)OH, C₁₋₆alkylS(O) (sulfoxide), nitroso, and C₁₋₆alkylOSO₂
alternatively, L, NR¹, and R², together with the phenyl attached to NR¹, form an optionally substituted bicyclic ring,
and
R^{m} in each instance is independently hydrogen or C₁₋₆alkyl or halo-C₁₋₆alkyl.

2. The compound of formula (I) or the pharmaceutically acceptable salt thereof of claim 1,
Wherein R¹ is hydrogen or methyl.
The compound of formula (I) or the pharmaceutically acceptable salt thereof of any one of claims 1-2, wherein X is fluorine or chlorine.

3. The compound of formula (I) or the pharmaceutically acceptable salt thereof of any one of claims 1-3, wherein R² and R³ are independently selected from the group consisting of C₁₋₄alkyl, CN, halo-C₁₋₄alkyl and halogen.

4. The compound of formula (I) or the pharmaceutically acceptable salt thereof of any one of claims 1-4, wherein at least one of R², R³, R⁴, R⁵, and R⁶ is halo-C₁₋₄alkyl or halogen.

5. The compound of formula (I) or the pharmaceutically acceptable salt thereof of any one of claims 1-5, wherein L is CH₂, CHCH₃, CO, or SO2.

6. The compound of formula (I) or the pharmaceutically acceptable salt thereof of any one of claims 1-6, wherein R² and R³ are independently C₁₋₃alkyl or halo-C₁₋₄alkyl.

7. The compound of formula (I) or the pharmaceutically acceptable salt thereof of any one of claims 1-7, wherein L is CO.

8. The compound of formula (I) or the pharmaceutically acceptable salt thereof of any one of claims 1-8, wherein L is SO2.

9. The compound of formula (I) or the pharmaceutically acceptable salt thereof of any one of claims 1-9, wherein wherein L and NR¹, together with the phenyl attached to NR¹, form a bicyclic ring.

10. The compound of formula (I) or the pharmaceutically acceptable salt thereof of any one of claims 1-10, wherein Ar is phenyl optionally substituted with one or more substituents selected from the group consisting of C₁₋₄alkyl OC₁₋₄alkyl, CN, halogen, N(R^{m})₂, C(O)OR^{m}, and C(O)N(R^{m}).

11. The compound of formula (I) or the pharmaceutically acceptable salt thereof of any one of claims 1-10, wherein Ar is pyridine or pyrimine, wherein the pyridine or pyrimine is optionally substituted with one or more substituents selected from the group consisting of C₁₋₄alkyl OC₁₋₄alkyl, CN, halogen, N(R^{m})₂, C(O)OR^{m}, and C(O)N(R^{m}).

12. The compound of formula (I) or the pharmaceutically acceptable salt thereof of any one of claims 1-10, wherein Ar is heterocyclyl selected from the group consisting of piperidine, piperazine, and morpholine, wherein the heterocyclyl is optionally substituted with one or more substituents selected from the group consisting of C₁₋₄alkyl OC₁₋₄alkyl, CN, halogen, N(R^{m})₂, C(O)OR^{m}, and C(O)N(R^{m}).

13. The compound of formula (I) or the pharmaceutically acceptable salt thereof of any one of claims 1-10, wherein R⁷ is phenyl optionally substituted with one or more substituents selected from the group consisting of C₁₋₄alkyl OC₁₋₄alkyl, CN, halogen, N(R^{m})₂, C(O)OR^{m}, and C(O)N(R^{m}).

14. The compound of formula (I) or the pharmaceutically acceptable salt thereof of any one of claims 1-10, wherein R⁷ is pyridine or pyrimine, wherein the pyridine or pyrimine is optionally substituted with one or more substituents selected from the group consisting of C₁₋₄alkyl OC₁₋₄alkyl, CN, halogen, N(R^{m})₂, C(O)OR^{m}, and C(O)N(R^{m}).

15. The compound of formula (I) or the pharmaceutically acceptable salt thereof of any one of claims 1-14, wherein one ore two of R², R³, R⁴, R⁵ and R⁶ are CF₃.

16. The compound of formula (I) or the pharmaceutically acceptable salt thereof of claim 1, wherein the compound is selected from the group consisting of

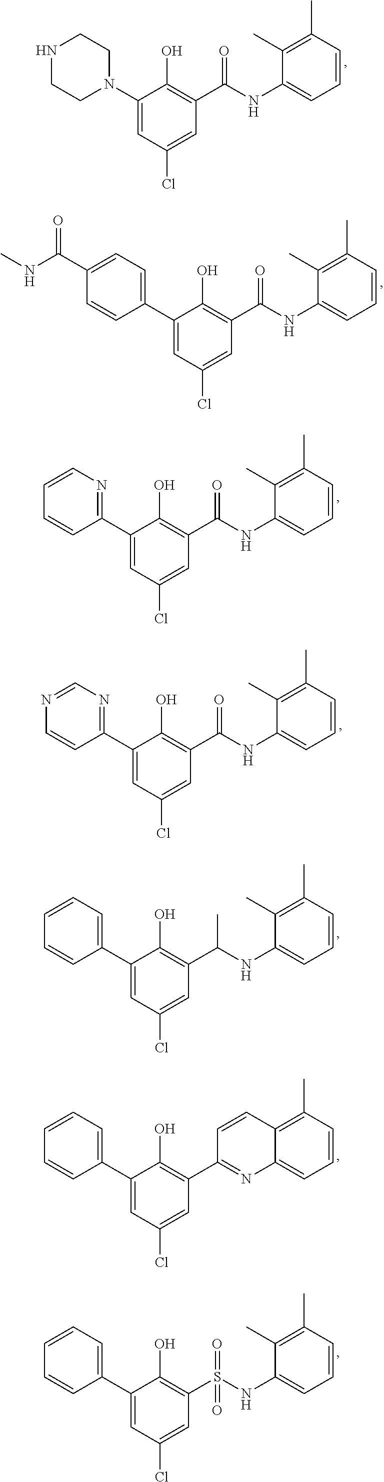

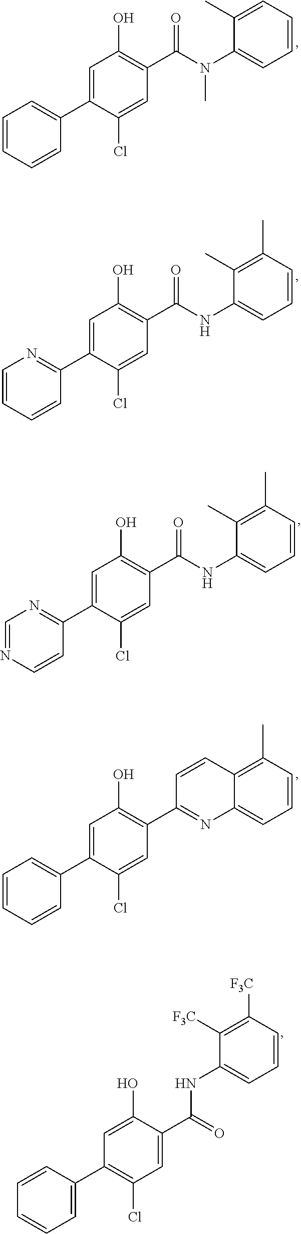 [SMILES: Cc1ccccc1N(C)C(=O)c1cc(Cl)c(-c2ccccc2)cc1O ; Cc1cccc(NC(=O)c2cc(Cl)c(-c3ccccn3)cc2O)c1C ; Cc1cccc(NC(=O)c2cc(Cl)c(-c3ccncn3)cc2O)c1C ; Cc1cccc2nc(-c3cc(Cl)c(-c4ccccc4)cc3O)ccc12 ; O=C(Nc1cccc(C(F)(F)F)c1C(F)(F)F)c1cc(Cl)c(-c2ccccc2)cc1O]

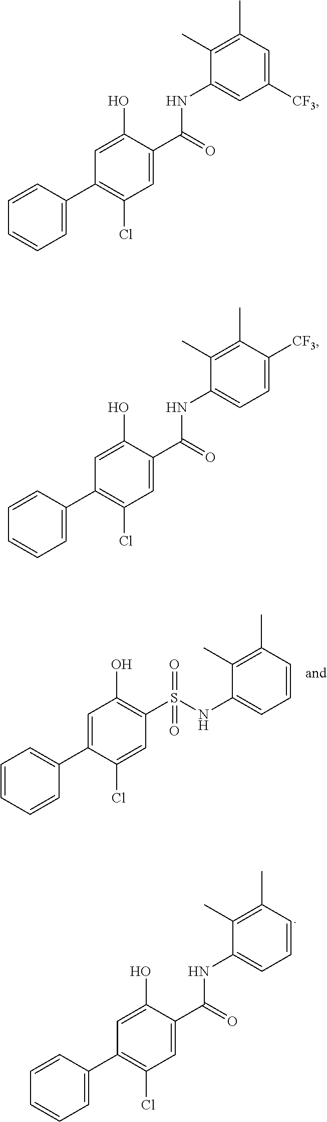 [SMILES: Cc1cc(C(F)(F)F)cc(NC(=O)c2cc(Cl)c(-c3ccccc3)cc2O)c1C ; Cc1c(NC(=O)c2cc(Cl)c(-c3ccccc3)cc2O)ccc(C(F)(F)F)c1C ; Cc1cccc(NS(=O)(=O)c2cc(Cl)c(-c3ccccc3)cc2O)c1C ; Cc1cccc(NC(=O)c2cc(Cl)c(-c3ccccc3)cc2O)c1C]

17. A pharmaceutical composition comprising the compound of formula (I) or the pharmaceutically acceptable salt thereof of any one of claims 1-16 and a pharmaceutically acceptable carrier.

18. A method of treating a disease associated with abnormal mitochondrial fusion and/or mitochondrial fission, comprising administering to a subject in need thereof a therapeutically effective amount of the compound of formula (I) or the pharmaceutically acceptable salt thereof of any one of claims 1-16 or the pharmaceutical composition of claim 17, wherein the disease is selected from the group consisting of neurodegenerative diseases, metabolic disease, cardiovascular diseases, autoimmune disease, and cancer.

19. The method of claim 20, wherein the disease is selected from the group consisting of Leukemia, Acute Lymphoblastic Leukemia (ALL), Acute Myeloid Leukemia (AML), Chronic Lymphoblastic Leukemia (CLL), Chronic Myelogenous Leukemia (CML), Lymphoma, Hodgkin Lymphoma, Non-Hodgkin Lymphoma, Lung cancer, Non-Small Cell Lung Cancer, Small Cell Lung Cancer, Melanoma, Prostate cancer, Brain Tumors, Breast Cancer, Bladder Cancer, Colorectal Cancer, Head and Neck Cancer, Ovarian Cancer, Pancreatic Cancer, Hepatocellular (Liver) Cancer, Renal Cell (Kidney) Cancer, Skin Cancer, Ewing Sarcoma, Rhabdomyosarcoma, Osteosarcoma, Soft tissue sarcoma, obesity, mitochondrial disorder, diabetes, insulin resistance, sarcopenia, acute liver failure, liver fibrosis, nonalcoholic steatohepatitis, hepatosteatosis, alcoholic fatty liver, kidney fibrosis, chronic kidney disease, Alzheimer's disease, amyotrophic lateral sclerosis, Friedreich ataxia, Huntington's disease, lewy body disease, parkinson's disease, spinal muscular atrophy, frontotemporal dementia, prion diseases, mitochondrial encephalopathy, age-related macular degeneration, glaucoma, diabetic retinopathy, and retinitis pigmentosa.

20. A method of inhibiting mitofusin-mediated mitochondrial fusion, comprising contacting a cell with an effective amount of the compound of formula (I) or the pharmaceutically acceptable salt thereof of any one of claims 1-16 or the pharmaceutical composition of claim 17 to inhibit mitofusin 1 and/or mitofusin 2.
